(19) United States
(12) Patent Application Publication
(10) Pub. No.: US 2008/0039392 A1
(43) Pub. Date: Feb. 14, 2008

(54) CRAC channel and modulator screening methods

(75) Inventors: Michael D. Cahalan, Newport Coast, CA (US); Shenyuan L. Zhang, Irvine, CA (US); Andriy V. Yeromin, Irvine, CA (US)

Correspondence Address: QUINE INTELLECTUAL PROPERTY LAW GROUP, P.C., P O BOX 458, ALAMEDA, CA, 94501, US

(73) Assignee: THE REGENTS OF THE UNIVERSITY OF CALIFORNIA, Oakland, CA (US)

(21) Appl. No.: 11/807,244

(22) Filed: May 24, 2007

Related U.S. Application Data
(60) Provisional application No. 60/808,733, filed on May 26, 2006.
(60) Provisional application No. 60/834,234, filed on Jul. 28, 2006.
(60) Provisional application No. 60/830,948, filed on Jul. 14, 2006.

(51) Int. Cl. A01K 67/00; A61K 31/70; A61P 35/00; C07K 19/00; C12N 15/00; C12N 5/04; C12Q 1/68; G01N 33/53

(52) U.S. Cl. 514/12; 435/325; 435/348; 435/352; 435/366; 435/455; 435/6; 435/7.1; 530/300; 530/387.1; 800/8

(57) ABSTRACT

Recent studies by our group and others demonstrated a required and conserved role of Stim in store-operated Ca²⁺ (SOC) influx and Ca²⁺ release-activated Ca²⁺ (CRAC) channel activity. Using an unbiased genome-wide RNAi screen in Drosophila S2 cells, 75 hits were identified that strongly inhibited Ca²⁺ influx upon store emptying by thapsigargin (TG). Among these hits are 11 predicted transmembrane proteins, including Stim and one, olf186-F, that upon RNAi-mediated knockdown exhibited a profound reduction of TG-evoked Ca²⁺ entry and CRAC current, and upon overexpression a three-fold augmentation of CRAC current. CRAC currents were further increased to eight-fold higher than control and developed more rapidly when olf186-F was co-transfected with Stim. olf186-F is a member of a highly conserved family of four-transmembrane spanning proteins with homologs from C. elegans to human. The ER Ca²⁺ pump SERCA and the SNARE protein Syntaxin5 were also required for CRAC channel activity, consistent with a signaling pathway in which Stim senses Ca²⁺ depletion within the ER, translocates to the plasma membrane, and interacts with olf186-F to trigger CRAC channel activity.

### CROSS REFERENCE TO RELATED APPLICATIONS

This application claims priority to U.S. Ser. No. 60/808,733 “Methods for Identifying Genes That Regulate CRAC Channel Activity” by Cahalan et al., filed May 26, 2006; 60/830,948 “Methods for Identifying Genes That Regulate CRAC Channel Activity” by Cahalan et al. filed Jul. 14, 2006; and 60/834,234 “CRAC Channel and Modulator Screening Methods” By Cahalan et al. filed Jul. 28, 2006. These prior applications are incorporated herein by reference for all purposes.

### GRANT INFORMATION

This invention was made in part with government support under Grant No. NS14609 awarded by the National Institutes of Health, by a fellowship from the George E. Hewitt Foundation, and by Scientist Development Grant 0630117N from the American Heart Association. The United States government may have certain rights in this invention.

### FIELD OF THE INVENTION

The invention relates generally to calcium release activated calcium (CRAC) channels and modulators thereof.

### BACKGROUND OF THE INVENTION

Calcium is a second messenger molecule in almost all cell types. In many different cells, repetitive oscillations of intracellular concentrations of Ca²⁺ ([Ca²⁺]ᵢ) result from the activation of the phosphoinositide signaling pathway through cell-surface receptors.

For example, Ca⁺⁺ influx across the cell membrane is important in lymphocyte activation and adaptive immune responses. [Ca²⁺]ᵢ oscillations triggered through stimulation of the TCR (T-cell antigen receptor) have been demonstrated to be prominent, and appear to involve only a single Ca²⁺-influx pathway, the store-operated CRAC channel (Ca²⁺-release-activated Ca²⁺ channel). See, e.g., Lewis (2001) “Calcium signaling mechanisms in T lymphocytes,” Annu. Rev. Immunol. 19, 497-521; Feske et al. (2003) “Ca⁺⁺ calcineurin signalling in cells of the immune system,” Biochem. Biophys. Res. Commun. 311, 1117-1132; Hogan et al. (2003) “Transcriptional regulation by calcium, calcineurin, and NFAT,” Genes Dev. 17, 2205-2232 (2003); Gallo et al. (2006) “Lymphocyte calcium signaling from membrane to nucleus,” Nature Immunol. 7, 25-32. Antigen recognition by B and T cells triggers phospholipase C activation, inositol-1,4,5-triphosphate (IP3) generation and the release of Ca⁺⁺ from endoplasmic reticulum (ER) stores. Depletion of these stores opens CRAC channels, a class of ‘store-operated’ Ca⁺⁺ channels with high selectivity for Ca⁺⁺ over monovalent cations, low single-channel conductance and an inwardly rectifying current-voltage (I-V) relationship. See also, Hoth et al. (1992) “Depletion of intracellular calcium stores activates a calcium current in mast cells,” Nature 355: 353-356. Zweifach & Lewis (1993) “Mitogen-regulated Ca2 [symbol] current of T lymphocytes is activated by depletion of intracellular Ca⁺⁺ stores.” Proc. Natl. Acad. Sci. USA 90:6295-6299; Parekh. & Putney (2005) “Store-operated calcium channels,” Physiol. Rev. 85: 757810 (2005); and Prakriya & Lewis (2003) “CRAC channels: activation, permeation, and the search for a molecular identity” Cell Calcium 33: 311-321 (2003). Sustained Ca⁺⁺ influx results in NFAT dephosphorylation by the calmodulin-dependent protein phosphatase calcineurin and promotes NFAT translocation to the nucleus.

Controlled [Ca²⁺]ᵢ oscillations can be created in certain cells by controlling the influx of Ca²⁺ through open CRAC channels using a ‘calcium clamp,’ thus enabling quantitative studies of the effects of oscillation amplitude and frequency on downstream events. Recently, patch-clamp experiments have identified the biophysical characteristics of CRAC channels in lymphocytes and other human cell types. Despite the acknowledged functional importance of store-operated Ca²⁺ (SOC) influx in cell biology and of CRAC channels for immune cell activation, the intrinsic channel components and signaling pathways that lead to channel activation were previously unidentified.

### SUMMARY OF THE INVENTION

This invention includes the discovery that Orai is the pore forming component of the CRAC channel. In addition, the combined role of Orai and Stim in forming a functional CRAC channel is taught. Accordingly, the invention includes compositions comprising Orai, as well as Orai and Stim. Genes that encode these polypeptides are also a feature of the invention, as are methods of identifying modulators of Orai and/or Stim.

In a first aspect, the invention includes a recombinant cell having a heterologous orai gene, preferably in conjunction with a heterologous stim gene. The cell can be any cell, and is suitably a cell in culture, such as a mammalian, human, rodent, insect or Xenopus cell. The cell can also be present in a non-human multi-cellular organism. The orai gene and the stim gene are expressed in the cell to produce heterologous Orai and heterologous Stim polypeptides. The heterologous orai gene and the heterologous stim gene are expressed in the cell and a heterologous Orai/Stim polypeptide complex is optionally formed in the cell, or in a membrane of the cell.

The complex can be isolated or recombinant. Thus, isolated or recombinant polypeptide complexes can include a recombinant Orai polypeptide or a recombinant Stim polypeptide, and typically also include an Orai polypeptide, a Stim polypeptide, a recombinant Orai polypeptide or a recombinant Stim polypeptide. In one embodiment, the complex includes a recombinant Orai polypeptide and a recombinant Stim polypeptide. The source for the Orai and Stim polypeptides and genes can be from any source, e.g., the heterologous orai and stim genes or encoded polypeptides are optionally derived from human genes. The complex can be the result of recombinant expression in a cell, and the complex can be isolated by any available method, including, e.g., by co-immunoprecipitation with an antibody. Antibodies against an Orai polypeptide or Orai-Stim complex are similarly a feature of the invention.

The invention also includes a knock out non-human animal comprising a defect in a native orai gene or a defect in native orai gene expression, or both. For example, the animal can be a double knock-out, deficient in endogenous Orai polypeptide expression and endogenous Stim polypeptide expression, e.g., an animal that expresses a heterologous Orai polypeptide and a heterologous Stim polypeptide. In one embodiment, the animal is a laboratory animal, e.g., a non-human mammal such as a mouse. For example, the animal can be a mouse and the heterologous orai gene can be derived from a human orai gene, e.g., where the mouse also includes a heterologous human stim gene. Thus, a double knock out animal that includes heterologous orai and stim genes from a clinically relevant (e.g., human) source is one feature of the invention.

In an additional aspect, the invention includes methods of identifying a compound that binds to or modulates an activity of an Orai polypeptide, or an Orai polypeptide/Stim polypeptide complex. The method includes: (a.) contacting a biological or biochemical sample comprising the polypeptide or complex with a test compound; and, (b.) detecting binding of the test compound to the polypeptide or complex, or modulation of the activity of the polypeptide or polypeptide complex by the test compound, thereby identifying the compound that binds to or modulates the activity of the polypeptide or complex.

In the method, either the biological sample can be moved into contact with the test compound, or the test compound moved into contact with the biological sample, or both the test compound and the sample can be moved into contact with each other. One or more biological sample that includes one or more Orai polypeptide or Orai/Stim polypeptide complex can be contacted with a plurality of test compounds. Binding of the test compounds to the polypeptide or polypeptide complex can be detected, or modulation of the activity of the polypeptide or complex by the test compounds can be detected, thereby identifying one or more compound that binds to or modulates the activity of the polypeptide or complex.

The plurality of test compounds optionally includes a plurality of pre-screened compounds. These can include, e.g., naturally occurring compounds, ions, small organic molecules, peptides, peptide mimetics, ion channel agonists, ion channel antagonists, ion channel enhancers, Ca⁺⁺ channel blockers, stretch-induced channel blockers, and the like. The test compound optionally enhances an activity of the polypeptide or complex, potentiates an activity of the polypeptide or complex, inhibits or blocks an activity of the polypeptide or complex, or the like.

The biological sample can be derived, e.g., from a cell or tissue that expresses the polypeptides or complexes noted herein. Alternately, the biological sample can include purified polypeptides or complexes. The methods can include recombinantly expressing a orai gene in a recombinant cell, or both a orai gene and a stim gene in a recombinant cell, e.g., where the biological sample is derived from the recombinant cell. As in the compositions noted above, the orai gene, or the orai and the stim gene is/are heterologous to the recombinant cell (e.g., a mammalian, human, rodent insect cell or Xenopus cell; the cell can be a cell in culture or a primary cell). Optionally, the Orai polypeptide or the Stim/Orai polypeptide complex can be incorporated into a biosensor such as a device that includes a Chem-FET.

In the methods, any of the above features relating to the polypeptides and complexes already noted are applicable, e.g., the polypeptide is optionally expressed from a human Orai gene or homolog thereof, or the complex is optionally derived from a human orai gene or homolog thereof and a human stim gene or homolog thereof, etc.

Binding can be detected between the Orai polypeptide and a moiety such as a Stim polypeptide, a potentiator of the Orai polypeptide, an antagonist of the Orai polypeptide, an agonist of the Orai polypeptide, an inverse agonist of the Orai polypeptide, a ligand that specifically binds to the Orai polypeptide, an antibody that specifically binds to the Orai polypeptide and an antibody that specifically binds to the Orai/Stim complex. Similarly, binding can be detected between the Stim/Orai polypeptide complex and a moiety selected from the group consisting of: a potentiator of the complex, an antagonist of the complex, an agonist of the complex, an inverse agonist of the complex, a ligand that specifically binds to the complex, and an antibody that specifically binds to the complex. Detection can be performed in vitro, in situ or in vivo. Additionally, a signal resulting from the activity of the Orai polypeptide or the Stim/Orai polypeptide complex can be detected. These signals include, e.g., a conformation-dependent signal, e.g., where a conformation of the Orai polypeptide or the Stim/Orai polypeptide complex is modified by binding of the test compound to the Orai polypeptide or to the Stim/Orai polypeptide complex.

Detecting binding of the test compound to the Orai polypeptide or the Stim/Orai polypeptide complex, or activity of the test compound on the Orai polypeptide or the Stim/Orai polypeptide complex can include detecting one or more of: binding between Stim and Orai, formation or stability of the polypeptide complex, Ca²⁺ flux, cytosolic calcium concentration, capacitive calcium entry, ion flux, changes in an activity of an intracellular ion sensor, depolarization of the cell, cell membrane voltage changes, cell membrane conductivity changes, calcium kinase activity triggered upon binding of a compound to the Orai polypeptide, generation, breakdown or binding of a phorbol ester by the Orai polypeptide, binding of diacylglycerol or other lipids by the Orai polypeptide, cAMP activity, cGMP activity, GTPgammaS binding, phospholipase C activity, activity of an enzyme involved in cellular ionic balance, binding of Orai to another protein, or a transcriptional reporter activity.

In a related aspect, the invention includes a system for detecting compounds that bind to or modulate an activity of an Orai polypeptide or Orai/Stim polypeptide complex. The system includes any of the features noted above, e.g., (a.) a biological sample comprising the polypeptide or the polypeptide complex; (b.) a source of a plurality of test compounds; and, (c.) a detector capable of detecting binding of one or more of the test compounds to the polypeptide or polypeptide complex, or modulation of the activity of the polypeptide or complex by one or more of the test compounds, thereby identifying a compound that binds to or modulates the activity of the polypeptide or complex.

Each of the features noted above with respect to the biological sample, compositions, cells, etc., are applicable to the detection system as well. For example, the source of test compounds optionally includes a library of compounds such as a pre-screened library of compounds.

The detector can include any detector configured to detect an appropriate signal in the system. For example, in cell based assays, the system can include a patch clamp or optical detection device. In one example, the detector includes a fluorescence detector that detects fluorescence, FRET, calcium concentration, changes in membrane potential or flow of a dye into or out of the cell.

In an additional aspect, the invention includes methods of detecting a molecular basis for an orai gene abnormality. The methods can include, e.g., determining whether a biological sample from a patient comprises a polymorphism in a gene encoding Orai or an abnormality in expression of Orai; and, correlating the polymorphism with an abnormality. Similarly, abnormalities in both an orai gene and a stim gene can be detected and correlated with an abnormality. Any type of polymorphism can be detected, e.g., a single nucleotide polymorphism, altered gene expression, etc.

The invention also provides methods of rescuing cells that have altered or missing Orai function. The methods include introducing a nucleic acid into the cell, where the nucleic acid encodes a recombinant polypeptide homologous to a natural Orai polypeptide. The recombinant polypeptide is expressed, providing Orai function to the cell. The cell can be in culture (e.g., for in vitro screening applications), in a tissue (e.g., for in situ screening applications) or in vivo.

A mutant Orai polypeptide that has an alteration in ion selectivity is also a feature of the invention. For example, the mutant can be a dominant negative mutation. In one specific example, the mutant is selective for monovalent cations (rather than Ca²⁺). In one example, the mutant is outwardly rectifying. Specific examples include a glutamate-aspartate mutation or an E to A mutation at a position corresponding to position 180 in a protein encoded by olf-186-f. This mutant includes a mutation at an amino acid position corresponding to amino acid 180 within the S1-S2 loop of olf-186-f Nucleic acids that encode this polypeptide are also a feature of the invention.

The present invention includes a demonstration that monitoring Ca²⁺ influx can be used to identify genes that regulate CRAC channel activity. Accordingly, the invention provides methods of identifying genes that regulate CRAC channel activity in a cell, e.g., by contacting the cell with an agent that inhibits SERCA pump-mediated reuptake of Ca²⁺ into cellular stores. The cell is then contacted with an RNAi molecule such as an individual dsRNA amplicon. A detected change in Ca²⁺ influx as compared to Ca²⁺ influx of the cell prior to contacting with the agent is indicative of a gene that regulates CRAC channel activity. In one embodiment, Ca²⁺ influx is reduced. The agent is optionally thapsigargin (TG).

The present invention further relates to a method of identifying an agent that blocks calcium flux through a calcium channel. The method includes contacting a cell containing a calcium channel gene with an agent, contacting the cell with an RNAi molecule, and then detecting a change in calcium flux as compared to calcium flux of the cell prior to contacting with the agent. A reduction in calcium flux is indicative of an agent that blocks calcium flux through a calcium channel gene. In one embodiment, the gene is olf186-F. In another embodiment, contacting with an RNAi molecule includes contacting the cell with an individual dsRNA amplicon, wherein the amplicon is prepared from one or more primer pairs as provided in Table 4.

The present invention further relates to a method of treating an immunological disorder, such as SCID in a subject by administering to the subject a therapeutically effective amount of a protein identified as a SOC regulator, which increases the Ca²⁺ influx in cells of the subject. In one embodiment, the protein is an expression product of a gene identified by the methods of the invention. In another embodiment, the protein is produced from a gene selected from the group of genes listed in Table 2.

The present invention further relates to a method of treating a cell proliferative disorder, such as cancer in a subject by administering to the subject a therapeutically effective amount of a protein identified as a SOC regulator, which increases the Ca²⁺ influx in cells of the subject. In one embodiment, the protein is an expression product of a gene identified by the methods of the invention. In another embodiment, the protein is produced from a gene selected from the group of genes listed in Table 2.

### BRIEF DESCRIPTION OF THE DRAWINGS

FIGS. 1A-1C provide graphical diagrams showing identification of genes involved in store-operated calcium entry. Panel A shows the effect of individual gene silencing on TG-evoked Ca²⁺ entry (CCE) relative to basal Ca²⁺, displayed as a histogram. The inset shows the distribution of averaged CCE/basal values for each well. Low values of CCE/basal are enlarged to show the tail of the distribution, representing amplicons that dramatically suppressed TG-evoked calcium entry. Panel B shows the top 10 hits with strongest effect on TG-evoked Ca²⁺ influx. Averaged values of CCE/basal are shown for all 48,384 wells tested in the assay (“mean”); for the top 10 hits from the screen; and for the positive control well that contained Stim dsRNA in each assay plate (“Stim Ave”). Striped bars represent hits with transmembrane regions. Panel C shows transmembrane (TM) protein hits.

FIGS. 2A-2F are pictorial and graphical diagrams showing suppression of TG-dependent Ca²⁺ influx and CRAC current by olf186-F dsRNA. Panel A shows Reduction of olf186-F mRNA expression in olf186-F dsRNA-treated cells. RT-PCR analysis on olf186-F, Stim, CG11059 and a control gene, Presenilin (Psn). Panel B shows [Ca²⁺]ᵢ in eight representative S2 cells treated with CG11059 dsRNA. Solution exchanges are indicated. Panel C shows [Ca²⁺]ᵢ in eight cells treated with olf186-F dsRNA. Panel D shows Averaged [Ca²⁺]ᵢ values ±SEM for control cells (n=195 cells in three experiments, white bars) and olf186-F dsRNA-treated cells (n=189 in four experiments, grey bars): resting [Ca²⁺]ᵢ; peak value upon readdition of 2 mM external Ca²⁺ before TG treatment (Ca0→Ca2); peak [Ca²⁺]ᵢ during TG-evoked release transient (Ca0+TG); maximal and sustained (3 min) [Ca²⁺]ᵢ after readdition of 2 mM external Ca²⁺. Panel E shows representative time course of whole-cell currents recorded in control cells treated with CG11059 dsRNA and in cells treated with olf186-F dsRNA. Panel F shows suppression of CRAC current by olf186-F dsRNA pretreatment. Each point represents the maximal inward CRAC current density (pA/pF) in a single cell, plotted as absolute values in consecutive order from left to right within three groups of cells: untreated; cells treated with dsRNA to suppress CG11059; or olf186-F (P<5*10⁻⁶ compared to either control group). The untreated cell group includes two cells each with current density >12 pA/pF. Horizontal lines indicate the mean value of current density in each group.

FIG. 3A-3F are graphical diagrams showing overexpression of olf186-F leads to increased CRAC currents in S2 cells. Panel A shows Representative CRAC currents in S2 cells transfected with GFP only (control), Stim, olf186-F, and olf186-F plus Stim. Panel B shows Ca²⁺ current in olf186-F+Stim co-transfected cell. Arrows a and b indicate the time corresponding to current-voltage curves in C. Panel B shows current-voltage relationship of CRAC current in the same cell. Panel D shows CRAC current density in transfected S2 cells, plotted as in FIG. 2F, within four groups of cells: GFP-transfected control; Stim and GFP co-transfected (not significantly different from controls); olf186-F and GFP co-transfected (P<10⁻³); olf186-F, Stim and GFP co-transfected (P<5*10⁻⁶). Panel E shows results of a method to analyze kinetics of CRAC current development. Panel F shows effect of co-transfected Stim on delay kinetics. Delay times are significantly reduced (P<5*10⁻⁶), but time_{1/2} values are not altered when Stim is expressed together with olf186-F, compared to olf186-F alone.

FIGS. 4A-4F are graphical diagrams showing the effects of Ca-P60A dsRNA on Ca²⁺ dynamics in individual S2 cells. Panel A shows averaged [Ca²⁺]ᵢ in cells treated with control CG11059 dsRNA. Panel B shows averaged [Ca²⁺]ᵢ in cells treated with Ca-P60A dsRNA. Panels C and D show Ca²⁺ release evoked by 1 μM ionomycin in control cells and in cells treated with Ca-P60A dsRNA to knock down SERCA expression. Panel E shows Averaged [Ca²⁺]ᵢ values ±SEM for control cells (white bars) and Ca-P60A dsRNA-treated cells (grey bars): labeled as in FIG. 2D, and including peak [Ca²⁺]ᵢ during ionomycin-evoked release transient (Ca0+Iono). Panel F shows Summary of inward CRAC current densities in control CG11059- and Ca-P60A dsRNA-treated cells (P=0.002); same plotting format as in FIG. 2F.

FIGS. 5A-5E are graphical diagrams showing the suppression of Ca²⁺ influx and CRAC current by Syx5 and tsr dsRNA. Averaged [Ca²⁺]ᵢ in cells treated with control CG11059 dsRNA (Panel A), Syx5 dsRNA (Panel B), or tsr dsRNA (Panel C). Panel D shows averaged [Ca²⁺]ᵢ values ±SEM for control cells (white bars), Syx5 dsRNA-treated cells (grey bars), and tsr dsRNA-treated cells (black bars): labeled as in FIG. 2D. Panel E provides a summary of inward CRAC current densities in Syx5 and tsr dsRNA-treated cells; same plotting format as in FIG. 2F. Mean values for CG11059 and Syx5 are significantly different (P=0.004). The mean values for CG11059 and tsr are not significantly different (P=0.65).

FIGS. 6A-6D are pictorial diagrams showing validation of effective mRNA overexpression or knockdown. RT-PCR analysis was performed as described in below using genespecific primers (Table 4). Panel A shows overexpression of olf186-F. Panel B shows suppression of Ca-P60A. Panel C shows suppression of Syx5. Panel D shows suppression of tsr.

FIGS. 7A-7F are graphical diagrams showing biophysical and pharmacological properties of enhanced CRAC current following olf186-F+Stim co-transfection. Panel A shows time course of currents with 2 mM external Ca²⁺, and during subsequent exposure to divalent-free Na⁺- or Cs⁺-containing solution. Arrows indicate the time points for current-voltage curves presented in B. Panel B shows corresponding Ca²⁺, Na⁺, and Cs⁺ current-voltage relations. Panel C shows Ca²⁺ currents in response to voltage pulses ranging from −110 to +110 mV in 10 mV increments from the holding potential of 10 mV. Panel D shows corresponding current-voltage curves (not leaksubtracted) at beginning (squares) and end (circles) of pulses. Panel E shows an effect of 2-APB at indicated concentrations. Panel F shows that Gd³⁺ reversibly blocks the Ca²⁺ current.

FIGS. 8A-8C are pictorial and graphical diagrams showing that olf186-F is a member of a conserved gene family. Panel A is a Phylogram of the olf186-F family. Homologous proteins of the Drosophila olf186-F gene product were searched with Phi-blast. The abbreviation of organisms are Hs: human; Mm: mouse; Rn: rat; Cf: dog; Gg: chicken; Dr: zebra fish; Dm: fly; Ce: worm. There are three conserved gene subfamilies within mammals. Panel B shows a Kyte-Doolittle hydropathy plot (window=11 residues) of the Drosophila olf186-F gene product. The Y-axis represents hydrophobicity and the X axis represents the 351 amino acid linear polypeptide sequence. The four putative transmembrane segments represented in fly and human homologs are designated S1-S4; S0 indicates an additional predicted alpha-helical structure found uniquely in the fly sequence. Panel C shows a diagram of predicted transmembrane topology and sequence of Drosophila olf186-F. Positively charged residues are shown blue, negatively charged residues are red, and conserved histidines are green. Residues that are identical in fly and three human homologs are shown enlarged and bold.

FIGS. 9A-9C are pictorial and graphical diagrams showing data from a genome-wide RNAi screen for SOC influx. Panel A provides a schematic diagram showing the screening protocol and time-line. Panel B provides a scatter plot for the duplicate genome-wide screens. The two “CCE/basal” values derived for each amplicon are plotted on the X and Y axes to show the overall reproducibility. In Panel C, the z-score was derived from the averaged CCE/basal value of each well. All dsRNAs that inhibited TG evoked calcium entry with a z-score of less than −3 (lower dashed line) were selected as hits for further analysis.

FIG. 10A-10B provide a gel photo (10A) a histogram (10B). The figure shows that Orai interacts with Stim and generates increased store-operated currents in S2 cells. Panel A shows representative co-immunoprecipitation of Stim and Orai (n=3). INPUT: total cell lysate, showing equal amount of samples prepared for IP. Panel B shows activation of CRAC current (at −130 mV) by three methods: IP₃ (10 μM IP₃ added to high-Ca²⁺ pipette solution; current density −11.4±3.1 pA/pF, n=5); thapsigargin (high-Ca²⁺ pipette solution and TG added to bath; −7.6±2.5 pA/pF, n=2); or passive store-depletion (Ca²⁺-free pipette solution; −28.7±1.8 pA/pF, n=27). All three methods produced substantially greater CRAC current density than passive store-depletion in control cells (−2.5±0.4 pA/pF, n=18).

FIG. 11A-11F provide histograms, showing that mutation E180D in Orai alters ion selectivity of CRAC current. Panel A shows time courses of inward current at −130 mV and outward current at 90 mV in representative cells overexpressing wild-type (WT, black) or E180D mutant (red) Orai. Ca²⁺-free internal solution. Panel B shows I-V curves at times indicated in Panel A. Panel C shows I-V curves of WT Orai-induced current in Ca2 solution and in choline external solution with 1.1 mM Na⁺ and 2 mM Ca²⁺. Panel D shows representative I-V curves for E180D Orai with the same solutions (n=3). Divalent cation selectivity of WT Orai CRAC current. I-V curves normalized to current values at −130 mV in Ca2 solution. Test solutions contained 20 mM test divalent and 124 mM Na⁺. External solutions for e and f are labeled according to color. f, I-V curves (not leak-subtracted) for E180D Orai with the same divalent cations, normalized to currents at 90 mV in Ca2 solution.

FIG. 12A-12F provide histograms showing Monovalent current in the absence of divalent ions exhibits altered ion selectivity in the E180D Orai mutant. Panel A shows a time course of inward current in cell expressing WT Orai. Bars indicate external solution exchange. Panel B is similar to panel A, for E180D Orai. Panel C shows I-V curves for WT Orai at times indicated in Panel A. Panel D shows I-V curves for E180D Orai at times indicated in Panel B. Panel E provides I-V curves of E180D Orai-induced current in the presence of varying external [Ca²⁺]. Panel F provides [Ca²⁺] dependence of E180D CRAC current at −130 (squares) and 90 mV (circles), scaled to the current in divalent-free solution. Ca²⁺-dependent block was fitted by the function y=1/(1+[Ca²⁺]/IC₅₀), where IC₅₀ is the calculated half-blocking Ca²⁺ concentration for inward (48±13 μM) and outward (2.05±0.78 mM) current, respectively.

FIG. 13A-D provide histograms showing charge-neutralizing mutations in S1-S2. Panel A shows I-V curves in cell expressing E180A mutant (without Stim) compared to control. Panel B shows I-V curves for D184A, D186A and N188A Orai mutants. Panel C provides CRAC current density in transfected S2 cells. Each point represents the maximal CRAC current density (pA/pF) in a single cell, plotted as absolute values: GFP-transfected control; E180A (P=1.7×10⁻⁴ relative to control); E180A+Stim (P=8.2×10⁻³); WT Orai+Stim; E180D+Stim inward current; D184A+Stim; D186A+Stim; N188A+Stim; and E180D+Stim outward current. Panel D shows suppression of CRAC current by 5 nM Gd³⁺ in WT Orai; D184A; E180D; and D186A. Bars indicate time of Gd³⁺ application, dashed lines indicate the zero-current level.

FIG. 14A-B provide a sequence alignment of Orai sequences (Panel A) and gel photographs (Panel B) Orai sequence, mutants, and expression. Panel A shows partial protein sequence comparison shows an overall 67% identity (*) and 89% similarity (* and :) within the S1-S2 region of Drosophila Orai and its human homologs. Putative transmembrane regions and mutation sites are indicated. Panel B shows validation of effective mRNA overexpression. RT-PCR analysis was performed as described in Supplementary Methods using gene-specific primers. Overexpression of Stim with WT Orai, E180A, E180D, or D184A (left) and with WT Orai, D186A or N188A (right).

FIG. 15, panels A and B provide histograms. FIG. 15 shows a block of inward and outward CRAC current by divalent cations and gadolinium. Panel A shows the effect of divalent cations (20 mM) on E180D. Orai-induced inward current (at −130 mV) and outward current (at 90 mV), normalized to currents in 2 mM Ca²⁺. Number of cells indicated above bars. Ca²⁺ (P<5×10⁻⁶ for outward current relative to 2 mM Ca²⁺ control); Ba²⁺ (P=6×10⁻⁴ for outward current and 7×10⁻⁵ for inward current); Sr²⁺ (NS); Mg²⁺ (P=0.01 for outward current and 0.03 for inward current). The change in I-V shape with 20 mM external Ba²⁺ and increased inward current may result from incomplete block of inward Na⁺ current at negative potentials. Panel B shows suppression of CRAC current by 5 nM Gd³⁺ in WT Orai; E180D; D184A (P<5×10⁻⁶ compared to WT); D186A (P=8×10⁻⁵); and N188A (P=1.5×10⁻³). Number of cells indicated above bars.

FIG. 16, panels A and B provide histograms. FIG. 16 shows an effect of 2-APB on CRAC current. Panel A shows WT Orai-induced CRAC current. Bars indicate time of 2-APB application at indicated concentrations. Panel B shows the same as panel a for the outward E180D Orai-induced CRAC current. Similar results were obtained in three separate experiments.

FIG. 17 provides a sequence alignment of Orai proteins.

FIG. 18 provides an alignment of Stim proteins.

FIG. 19 provides an alignment of Stim proteins.

### DETAILED DESCRIPTION OF THE INVENTION

Orai is, herein, definitively assigned as the pore forming component of the CRAC channel. Mutations in the conserved S1-S2 loop of Orai were sufficient to change ion selectivity of the CRAC channel in cells expressing the Orai mutant. Current rectification was also altered in cells expressing the mutant orai gene.

Stim is a protein that senses luminal Ca⁺⁺ content. See also, Liou et al. (2005) “STIM is a Ca²⁺ sensor essential for Ca²⁺-store-depletion-triggered Ca²⁺ influx.” Curr Biol 15:1235-41; and Zhang et al. (2005) “STIM1 is a Ca²⁺ sensor that activates CRAC channels and migrates from the Ca²⁺ store to the plasma membrane.” Nature 437: 902-5.

Orai and Stim were found to co-immunoprecipitate following TG-induced Ca⁺⁺ store depletion (co-immunoprecipitation was substantially enhanced by TG treatment). This demonstrates that Orai and Stim form a complex in response to Ca²⁺ store depletion. Further, co-expression of Orai and Stim increase CRAC channel activity. Thus, Orai and Stim collectively interact to initiate CRAC channel activity.

Accordingly, compositions of the invention include Orai and/or Stim, e.g., co-expressed from genes heterologous to a given cell. The CRAC channel is fundamental to a variety of cellular processes that are relevant in a variety of clinically relevant settings—including immunological diseases, cancer, hypertension, and many others. Accordingly, with the identification of Orai as the ion recognition component of the CRAC channel, and the identification of Orai and Stim as collective activators of the CRAC channel, methods and systems for identifying modulators of the CRAC channel are also a significant feature of the invention. Other useful tools provided by the invention include knock-out recombinant laboratory animals (e.g., double knock out mice that have heterologous human orai and stim genes), which permit study of potential modulators of clinically relevant targets in an in vivo system. Methods of screening orai and stim genes for polymorphisms that correlate with disease predisposition are also a feature of the invention.

### DEFINITIONS

Before describing the present invention in greater detail, it is to be understood that this invention is not limited to particular technical or biological systems or components, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used in accordance with the definitions set out below.

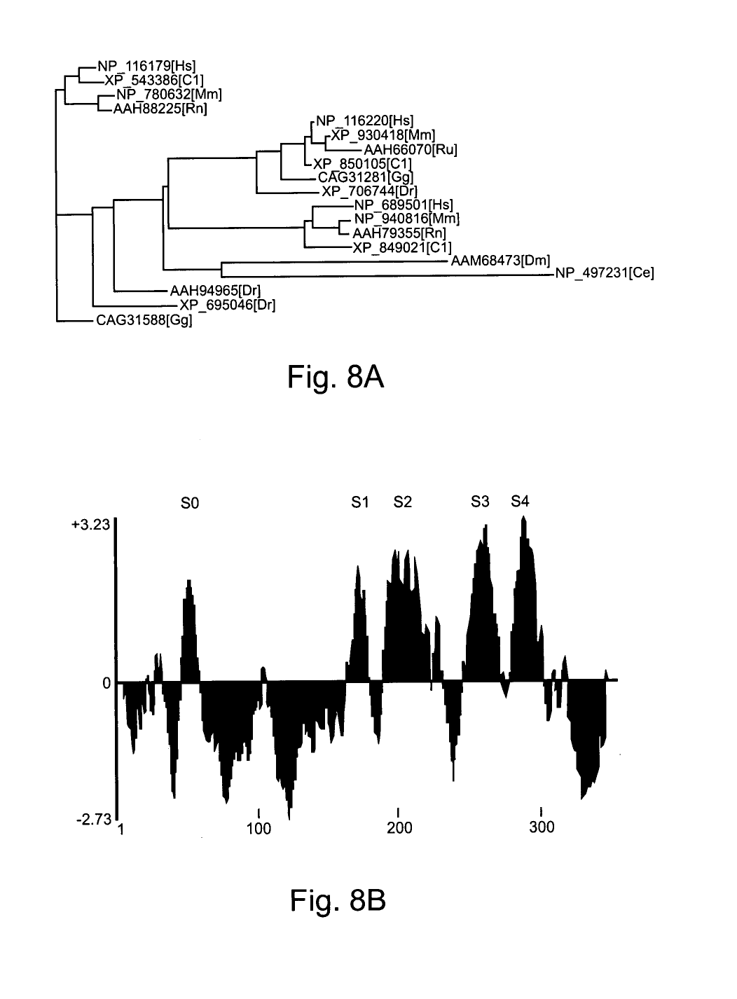

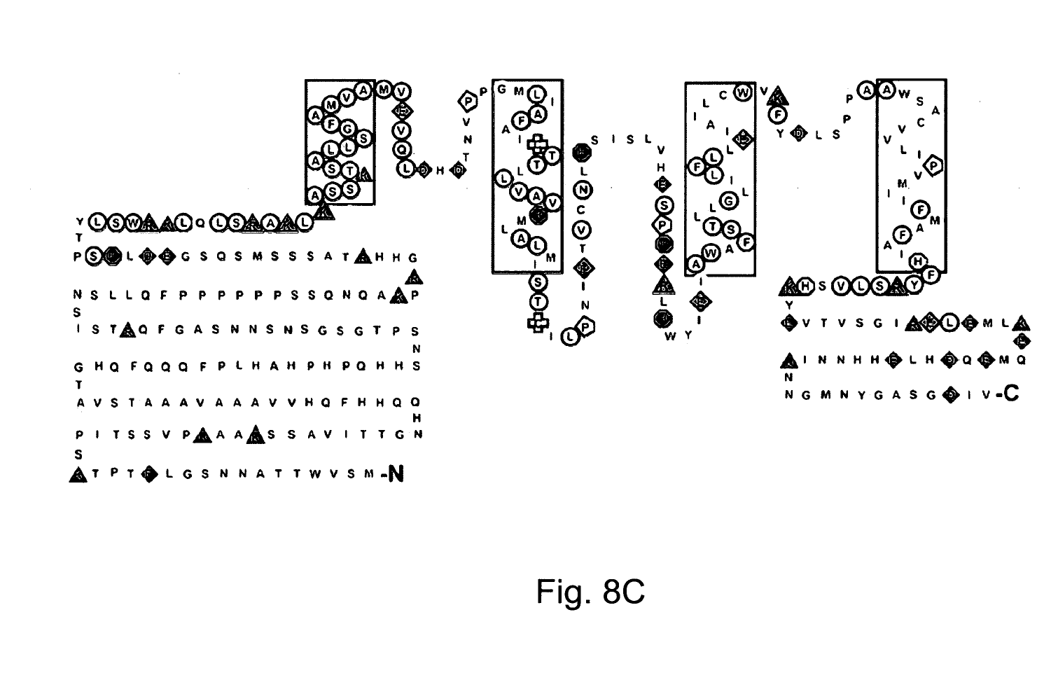

An “Orai polypeptide” is a polypeptide that is the same as, a splice-variant of, or homologous to a naturally occurring Orai polypeptide, or that is derived from such a polypeptide (e.g., through cloning, recombination, mutation, or the like). The polypeptide can be full length or can be a fragment of a full length protein. An Orai fragment typically includes at least 10 contiguous amino acids corresponding to a native Orai protein, such as an insect (e.g., olf 186) or human Orai protein. The Orai polypeptide is a member of a highly conserved gene family that includes three known homologs in mammals, two in chicken, three in zebrafish, and one member only in fly and worm (FIG. 8A, 14 and 17; Table 1A). The polypeptide can be naturally occurring or recombinant, and can be unpurified, purified, or isolated, and can exist, e.g., in vitro, in vivo, or in situ. In one typical useful embodiment, the polypeptide is a transmembrane protein. As described herein, in useful embodiments, the polypeptide can be a component of a CRAC channel protein, in any of a variety of contexts, including in cells of the immune system.

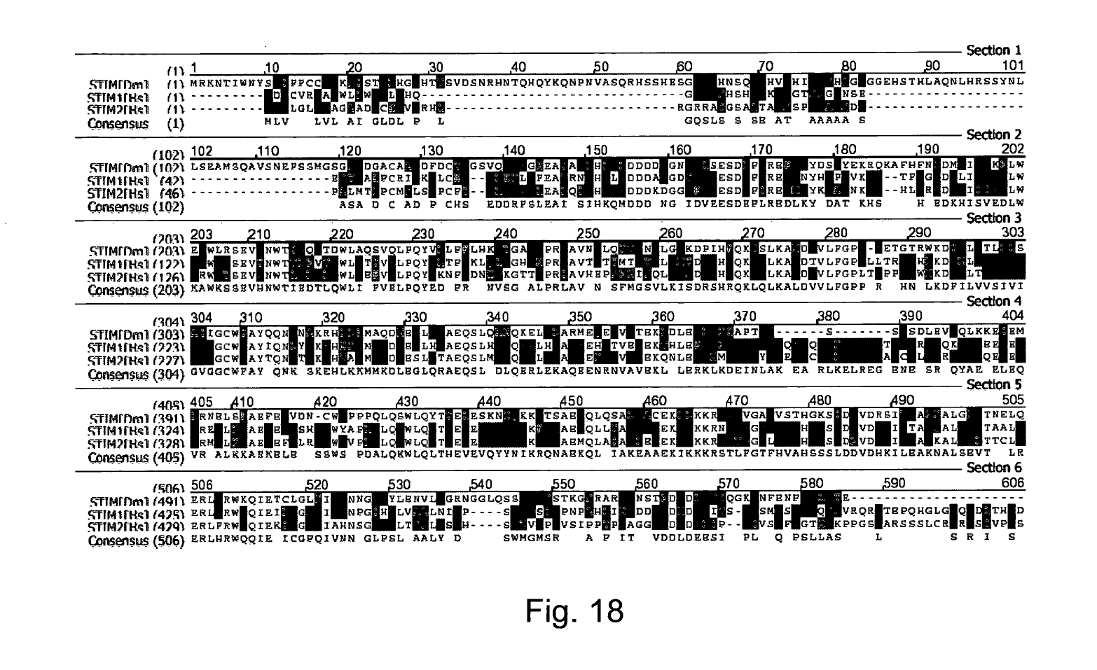

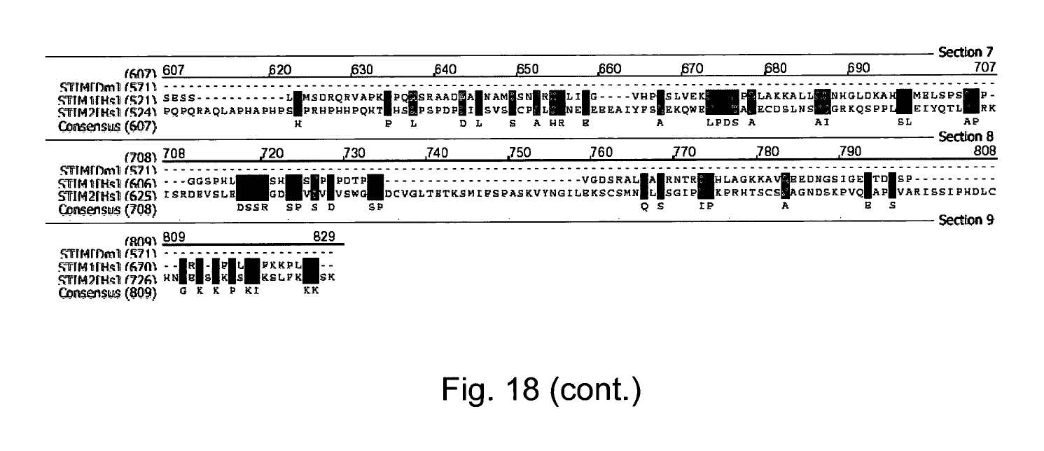

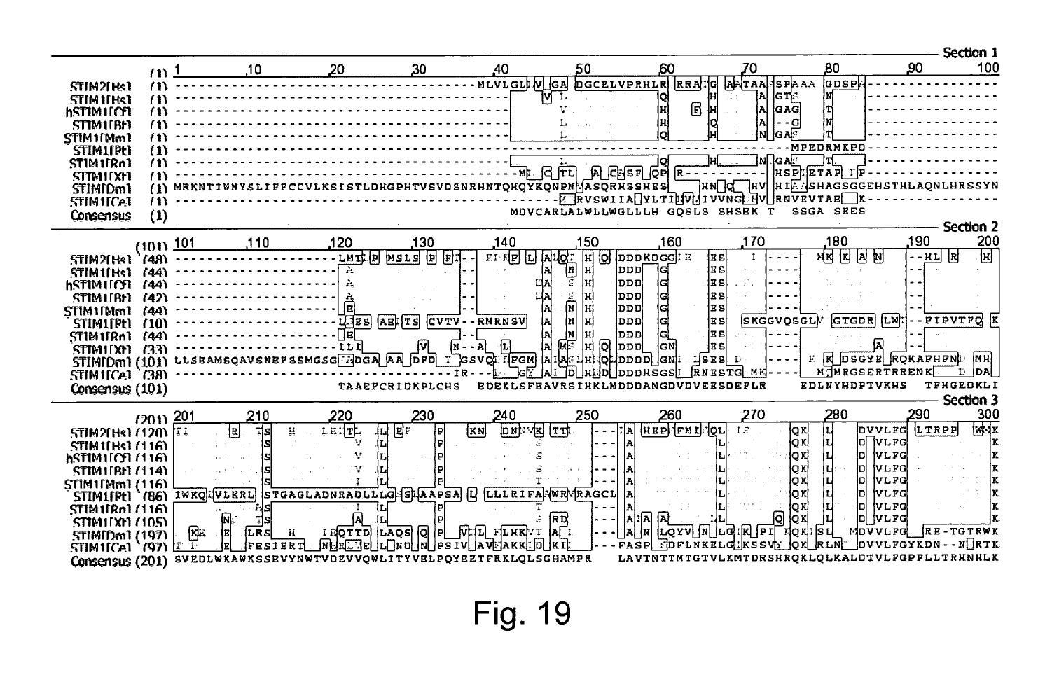

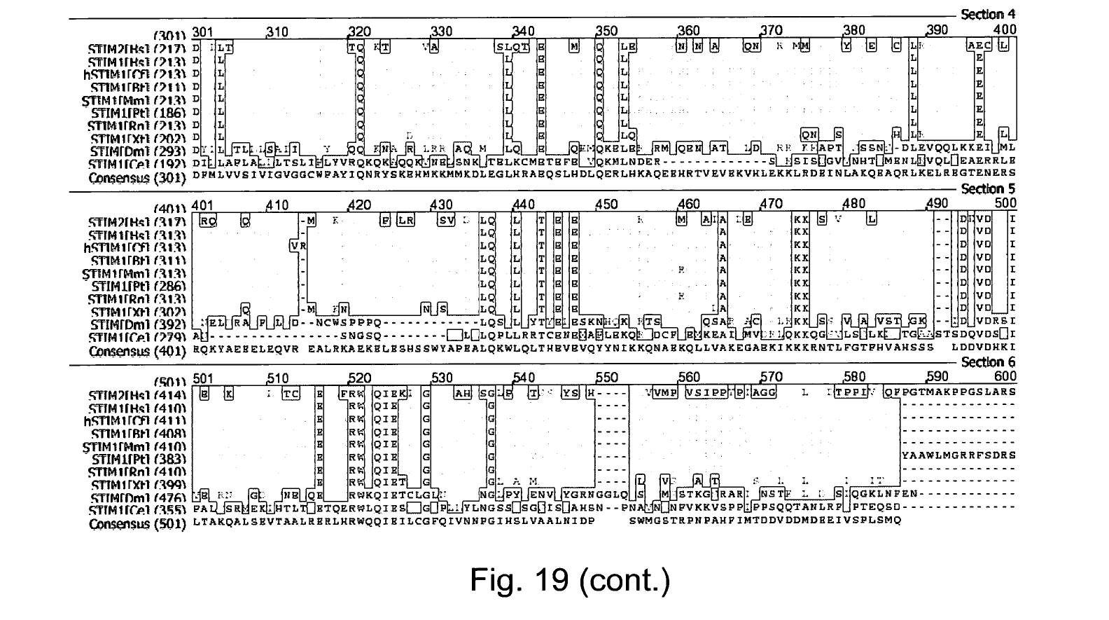

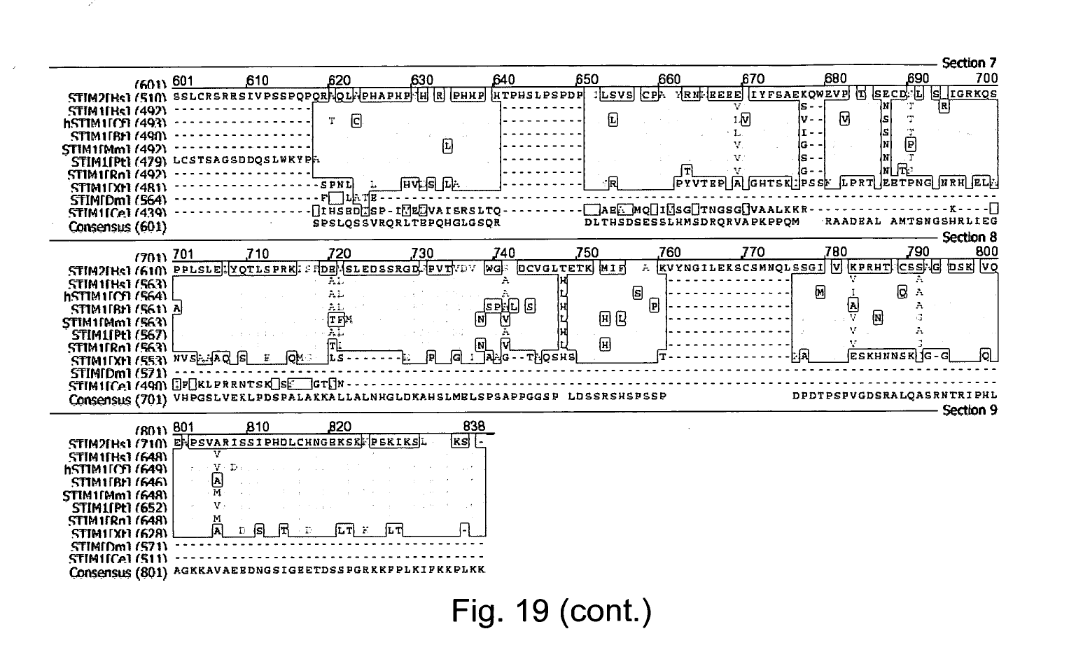

A “Stim polypeptide” is a polypeptide that is the same as, a splice-variant of, or homologous to a naturally occurring Stim polypeptide, or that is derived from such a polypeptide (e.g., through cloning, recombination, mutation, or the like). The polypeptide can be full length or can be a fragment of a full length protein. A Stim fragment typically includes at least 10 contiguous amino acids corresponding to a native Stim protein, such as an human Stim protein. Example Stim proteins are described in FIGS. 18 and 19. The polypeptide can be naturally occurring or recombinant, and can be unpurified, purified, or isolated, and can exist, e.g., in vitro, in vivo, or in situ. In one typical useful embodiment, the polypeptide is a transmembrane protein. As described herein, in useful embodiments, the polypeptide can be co-immunoprecipitated with an Orai protein from a biological sample, in any of a variety of contexts, including in cells of the immune system.

An orai gene is a nucleic acid that encodes an Orai polypeptide. Typically, the gene includes regulatory sequences that direct expression of the gene in one or more cells of interest. Optionally, the gene is a native gene that includes regulatory and coding sequences that naturally direct expression of an Orai polypeptide.

A stim gene is a nucleic acid that encodes a Stim polypeptide. Typically, the gene includes regulatory sequences that direct expression of the gene in one or more cells of interest. Optionally, the gene is a native gene that includes regulatory and coding sequences that naturally direct expression of a Stim polypeptide.

A biological sample comprising the Orai and/or Stim polypeptide includes any sample comprising the polypeptide or polypeptide complex that is derived from a biological source, e.g., cells, tissues, organisms, cells, secretions, etc. These samples can include, e.g., cells expressing the polypeptides or complexes, membranes containing the polypeptides or complexes, polypeptides or complexes bound to a chemical matrix, polypeptides or complexes bound to solid surface (e.g., for plasmon resonance), etc. A biochemical source can include biological sources and/or non-biological sources, such as purely synthetic preparations of materials.

A “modulator” is a compound that modulates an activity of a given polypeptide, polypeptide complex or receptor, e.g., an Orai and/or Stim polypeptide or polypeptide complex. The term “modulate” with respect to such a polypeptide or complex refers to a change in an activity or property of the polypeptide or complex. For example, modulation can cause an increase or a decrease in a protein activity, a binding characteristic (e.g., binding between Orai and Stim), membrane permeability of a Ca²⁺ ion of a membrane that comprises the polypeptide or complex, or any other biological, functional, or immunological property of such a polypeptide or complex. The change in activity can arise from, for example, an increase or decrease in expression of one or more genes that encode these polypeptides, the stability of an mRNA that encodes the polypeptide, translation efficiency, or from a change in activity of the polypeptide itself. For example, a molecule that binds to Orai or Stim or a complex thereof can cause an increase or decrease in a biological activity of the polypeptide. Example modulators include Orai or Stim allosteric enhancers, agonists, antagonists, inverse agonists, or partial agonists, Orai or Stim ligands, antibodies to Orai, Stim, or complexes thereof, etc. Antibodies to Orai-Stim complexes are described, e.g., in the Examples sections herein.

A “prescreened” compound is a compound that is pre-selected for a property of interest, such as toxicity, lack of toxicity, bioavailability, chemical structure, type of molecule (natural product, ion, ion channel agonist/antagonist/inverse agonist, etc.), or the like. For example, an “ingestible compound” is a compound that can be safely ingested in an amount that triggers a CRAC activity response by the compound. Certain compounds such as agonists or enhancers can have such a desired response when present at very low doses, while others are present in higher amounts.

A “transmembrane potential” is the work needed to move a unit of charge across a membrane such as a cell membrane.

A “cationic membrane permeable dye” is a dye which has a positive charge under specified pH (e.g., physiological pH) where the dye can cross a selected membrane such as the membrane of an intact cell. An “anionic membrane permeable dye” is a dye which has a negative charge at a specified pH (e.g., physiological pH) and which is membrane permeable and whose distribution between the inside and outside of the space bounded by the membrane or between the inside and outside of the membrane, depends on the transmembrane potential across the membrane. Similarly, a “neutral dye membrane permeable dye” is membrane permeable and has an overall neutral charge under the relevant conditions at issue, e.g., a specified pH (e.g., physiological pH).

A “voltage sensing composition” is a transmembrane potential indicator, e.g., comprising a fluorescent dye. Common voltage sensing compositions can include one or more cationic or anionic membrane permeable dye(s).

A membrane is “depolarized” when the transmembrane potential across the membrane becomes more positive inside. A membrane is “hyperpolarized” when the transmembrane potential becomes more negative inside.

A membrane is “permeable” to a given component (dye, ion, etc.) when that component can cross the membrane. Permeability can be dependent upon the relevant conditions, e.g., temperature, ionic conditions, voltage potentials, or the like.

Screening Test Compounds for Activity Against Orai and/or Stim

In one aspect, methods of identifying a compound that binds to or modulates an activity of a Orai and/or Stim polypeptide (or complex) are provided. In these methods, a biological or biochemical sample comprising the polypeptide or complex (e.g., recombinant Orai and/or Stim polypeptide) is contacted with a test compound and binding of the test compound to the polypeptide or complex, or modulation of the activity of the polypeptide or complex by the test compound is detected, thereby identifying a compound that binds to or modulates the activity of the polypeptide or complex. Compounds identified by these methods are also a feature of the invention.

Desirably, a test compound can be, e.g., a potentiator or enhancer of the polypeptide or complex, an antagonist of the polypeptide or complex, an agonist of the polypeptide or complex, an inverse agonist of the polypeptide or complex, a ligand that specifically binds to the polypeptide or complex, an antibody that specifically binds to the polypeptide or complex, or the like.

Additional Details Regarding Screening Methods

High throughput methods of screening are particularly useful in identifying modulators of Orai or Stim polypeptide activity, and/or of orai or stim gene expression. Generally in these methods, one or more biological sample that includes one or more orai or stim gene, Orai or Stim polypeptide, or complex thereof, is contacted with a plurality of test compounds. Binding to or modulation of the polypeptide or gene by the test compounds is detected, thereby identifying one or more compound that binds to or modulates activity of the polypeptide, complex and/or gene.

Essentially any available compound library can be screened in such a high-throughput format against a biological or biochemical sample, such as a cell expressing a Orai or Stim polypeptide, and activity of the library members against the polypeptide or expression thereof can be assessed, optionally in a high-throughput fashion.

Many libraries of compounds are commercially available, e.g., from the Sigma Chemical Company (Saint Louis, Mo.), Aldrich chemical company (St. Louis Mo.), and many can be custom synthesized by a wide range of biotech and chemical companies.

In one desirable aspect, a plurality of test compounds to be tested for activity against Orai, Stim, complexes thereof, or coding nucleic acids thereof, comprise a plurality of compounds. Thus, a library of test compounds to be screened for modulatory activity can include a previously unscreened library of compounds, or can include a pre-screened library of compounds that is pre-screened for any property that is desired, e.g., toxicity, bioavailability, chemical structure, known activity (e.g., ion channel binding or modulating activity) ingestibility, or the like. Further details on available libraries are found below.

In general, test compounds that enhance or potentiate an activity of the Orai or Stim polypeptide or complex can be desirable, e.g., to modulate calcium levels in a cell, which can influence any of a variety of downstream biological processes, including those described herein.

Additional Details Regarding Assay Formats

In one aspect, the present invention relates to the use of the Orai or Stim polypeptides, complexes thereof, and/or coding nucleic acids in methods for identifying a compound, that interacts/binds to the polypeptide. The test compound can be selected from natural or synthetic molecules such as ions, proteins or fragments thereof, carbohydrates, organic or inorganic compounds and/or the like. For example, the test compounds can be naturally occurring compounds, ions, small organic molecules, peptides, peptide mimetics, ion channel agonists, ion channel antagonists, ion channel enhancers, non-specific Ca⁺ channel blockers, Nifedipine and/or structurally related compounds, Verapamil and/or structurally related compounds, gadolinium and/or structurally related compounds, and/or stretch-induced channel blockers, etc.

This can be achieved, e.g., by utilizing the polypeptides or complexes of the invention, including active fragments thereof, in cell-free or cell-based assays. A variety of formats are applicable, including measurement of second messenger effects, e.g., Ca²⁺ flux, ion flux, depolarization of the cell, cell membrane voltage changes, cell membrane conductivity changes, kinase activity triggered upon binding of a compound to the polypeptide or complex, generation, breakdown or binding of a phorbol ester by the polypeptide or complex, binding of diacylglycerol or other lipids by the polypeptide, cAMP activity, cGMP activity, GTPgammaS binding, phospholipase C activity, activity of an enzyme involved in cellular ionic balance, binding of Orai or Stim to each other or to another protein, or a transcriptional reporter activity assay, e.g., using CRE, SRE, MRE, TRE, NFAT, and/or NFkB-response elements coupled to appropriate reporters.

For example, agonist-receptor interactions at the plasma membrane can cause production of inositol 1,4,5-trisphosphate (IP₃) in many cell types, which in turn releases Ca²⁺ from internal stores in the endoplasmic reticulum. Such a depletion of Ca²⁺ stores activates Ca²⁺ permeable store-operated ion channels (SOCs) in the plasma membrane, allowing a sustained Ca²⁺ influx termed “capacitative” or “stored-operated” Ca²⁺ entry. In some cell types, depletion of internal Ca²⁺ stores by either antigen/agonist binding or sarcoplasmic/endoplasmic reticulum Ca²⁺ (SERCA) pump inhibitors (e.g., thapsigagin (TG)), or dialysis of the cytosol by a whole-cell pipette solution during patch-clamp recordings activate a highly Ca²⁺-selective ion channel termed the Ca²⁺ release-activated Ca²⁺ (CRAC) channel, which has an extremely low unitary conductance for Ca²⁺ (24 fs). As noted herein, Orai forms the basic ion pore of the CRAC channel. Any of the steps leading to activation of the CRAC channel, or any downstream processes, can be monitored for effects of modulators on Orai and/or Stim.

Further Details Regarding Cell Free Assays

In one embodiment, cell-free assays for identifying such compounds comprise a reaction mixture containing an Orai or Stim polypeptide or complex encoded by orai and/or stim, or a variant thereof, and a test compound or a library of test compounds. Accordingly, one example of a cell-free method for identifying test compounds that specifically bind to an Orai polypeptide or a Stim polypeptide (or complex thereof) comprises contacting such a protein or functional fragment thereof with a test compound or library of test compounds and detecting the formation of complexes by conventional methods. Similarly, the effect on Orai/Stem complex formation by the test compound can also be determined by monitoring association of the proteins in the presence and absence of the test compound.

In one class of useful embodiments, the library of test compounds can be synthesized on a solid substrate, e.g., a solid surface, plastic pins or some other surface. The test compounds are reacted with the polypeptide(s) and washed to elute unbound polypeptide(s). Bound polypeptide(s) is/are then detected by methods well known in the art. A reciprocal assay can also be used, e.g., in which polypeptide is applied directly onto plates and binding of the test compound to the polypeptide(s) is detected. Antibody or ligand binding to the polypeptide(s) can also be detected in either format.

Interaction between molecules can also be assessed using real-time BIA (Biomolecular Interaction Analysis, e.g., using devices from Pharmacia Biosensor AB), which detect surface plasmon resonance (an optical phenomenon). Detection depends on changes in the mass concentration of macromolecules at the biospecific interface and does not require specific labeling of the molecules. In one useful embodiment, a library of test compounds can be immobilized on a sensor surface, e.g., a wall of a micro-flow cell. A solution containing the Orai or Stim polypeptide or complex thereof is then continuously circulated over the sensor surface. An alteration in the resonance angle, as indicated on a signal recording, indicates the occurrence of an interaction. This general technique is described in more detail in the BlAtechnology Handbook by Pharmacia.

Optionally, the Orai or Stim polypeptide is immobilized to facilitate separation of complexes between the polypeptide(s) and a test compound from uncomplexed forms of the polypeptide. This also facilitates automation of the assay. Complexation of Orai or Stim polypeptide (or of polypeptide complexes thereof) can be achieved in any type of vessel, e.g., microtitre plates, microfluidic chambers or channels, micro-centrifuge tubes and test tubes. In one embodiment, the Orai or Stim polypeptide is fused to another protein, e.g., glutathione-S-transferase to form a fusion protein which can be adsorbed onto a matrix, e.g., glutathione Sepharose™ beads (Sigma Chemical. St. Louis, Mo.), which are then combined with the test compound and incubated under conditions sufficient to form test-compound-polypeptide complexes. Subsequently, the beads are washed to remove unbound label, and the matrix is immobilized and the radiolabel is determined.

Similar methods for immobilizing proteins on matrices use biotin and streptavidin. For example, the protein can be biotinylated using biotin NHS N-hydroxy-succinimide), using well known techniques and immobilized in the well of streptavidin-coated plates.

Cell-free assays can also be used to identify compounds (e.g., potential CRAC channel modulators) that bind and/or modulate the activity of a Orai or Stim polypeptide or polypeptide complex. In one embodiment, the polypeptide or complex is incubated with a test compound and the transmembrane ion channel activity of the protein is determined. In another embodiment, the binding affinity of the protein to a target molecule is determined by standard methods.

Further Details Regarding Cell Based Assays

In addition to cell-free assays such as those described above, the Orai or Stim polypeptide, and/or complex thereof can desirably be used in cell-based assay for identifying compounds that bind to, activate and/or modulate Orai or Stim polypeptide or complex activity.

For example, one method for identifying compounds that bind to Orai or Stim polypeptides or complexes comprises providing a cell that expresses one or more of these proteins, e.g., a human Orai and/or Stim polypeptide, combining a test compound with the cell and measuring formation of a complex between the test compound and the human Orai or Stim polypeptide (or, e.g., between the test compound a polypeptide complex that includes both polypeptides). The cell can be a mammalian cell (e.g., a CHO cell), a yeast cell, a bacterial cell, an insect cell, a Xenopus oocyte, a human or other mammalian cell, an immune cell, a kidney cell or any other cell expressing the Orai or Stim polypeptide, whether that expression is natural to the cell or, more typically, the result of recombinant introduction of a heterologous orai or stim gene of interest into the cell. Further details regarding appropriate cells, sources of genes of interest, etc., are found herein.

In another embodiment, cells that naturally express Orai and Stim, such as immune cells (e.g., T cells), or, alternately, cells expressing heterologous Orai or Stim polypeptides or polypeptide complexes, or membrane preparations of such cells, can be utilized to screen for bioactivity of test compounds. The Orai polypeptides described herein are Ca²⁺ permeable cation selective channels (pore forming channels). Stim interacts with these polypeptides to provide CRAC channel activation, and co-immunoprecipitates with Orai. In addition, other proteins of interest, such as SH2 or SH3-domain adapter proteins, SNARE proteins, cytoskeletal proteins, etc., may interact with Orai polypeptides. A variety of intracellular effectors have been identified as being Ca²⁺/G-protein regulated including, but not limited to, Ca²⁺-induced intraorganellar Ca²⁺ release, adenyl cyclase, cyclic GMP, phospholipase C, phospholipase A2 and phosphodiesterases, calcineurin activation, nuclear relocalization of NFAT, etc.

Accordingly, the activity of intracellular effectors, and thus activity of Orai/Stim polypeptides/complexes, can also be measured by techniques that are well known. For example, the level of cAMP produced by activation of adenyl cyclase can be measured by assays which monitor cAMP, either in vivo by using FRET or transcriptional reporters sensitive to cAMP, or in vitro by directly measuring cAMP production. GTPase activity by G proteins can be measured, e.g., in plasma membrane preparations by measuring the hydrolysis of gamma ³²P GTP, or in vivo by FRET or by monitoring activity of downstream effectors such as PLC, adenylate cyclase, etc. Breakdown of phosphatidylinositol-4,5-bisphosphate to 1,4,5-IP3 and diacylglycerol can be monitored by measuring the amount of diacylglycerol using thin-layer chromatography, or measuring the amount of IP3 using radiolabeling techniques or HPLC, or in vivo by activation of the IP3 receptor and release of calcium from internal stores. The generation of arachidonic acid by the activation of phospholipase A2 can be readily quantitated by well-known techniques.

Efflux of intracellular calcium or influx of calcium from outside the cell, or capacitive calcium entry (e.g., following treatment with thapsigargin (TG)) can be measured using conventional techniques, e.g., loading cells with a Ca⁺⁺ sensitive fluorescent dye such as fura-2 or indol-1, and measuring any change in Ca⁺⁺ concentration using a fluorometer, such as Fluoskan Ascent Fluorescent Plate Reader or Fluorometric Imaging Plate Reader. Alternately, or in addition, patch clamp devices can be used to measure current density (e.g., pA/pF). The signal pathways initiated by Orai or Stim polypeptides or complexes in response to test compounds can also be monitored by reporter gene assays.

Assays that monitor changes in membrane potential by (1) voltage measurements in Xenopus oocytes injected with mRNA encoding Orai and/or Stim, (2) patch clamping in tissue culture cells expressing the receptor, and (3) fluorometric assays using voltage-sensitive dyes or ionic fluxes are preferred assays for monitoring membrane potential in the context of the present invention.

In other aspects, interactions between Orai or Stim and related proteins are monitored to detect activity or binding properties of Orai or Stim, or related complexes comprising Orai and/or Stim. Thus, in one aspect, interactions between Orai and Stim can be monitored. In addition, homodimers and heterodimers between Orai/Stim and other proteins can exist. Accordingly, binding between Orai/Stim and other proteins can be monitored, e.g., by FRET or other protein-protein interaction technologies (cross-linking, etc.) to monitor homodimer and heterodimer formation, gating by Orai and/or Stim or the like.

As described, other assays such as melanophore assays, Phospholipase C assays, Ca⁺⁺ mobilization assays, beta-arrestin FRET assays, and transcriptional reporter assays, e.g., using CRE, SRE, MRE, TRE, NFAT, and/or NFkB-response elements coupled to appropriate reporters can be used. Detection using reporter genes coupled to appropriate response elements are particularly convenient. For example, the coding sequence to chloramphenicol acetyl transferase, beta galactosidase or other convenient markers are coupled to a response element that is activated by a messenger molecule that is activated by a protein of the invention, e.g., through Ca⁺⁺ modulation. Cells expressing the marker in response to application of an appropriate test compound are detected by cell survival, or by expression of a colorimetric marker, or the like, according to well established methods.

Any of a variety of potential modulators of orai or stim gene, or Orai or Stim polypeptide activity or expression can be screened for. For example, potential modulators (ions, small organic molecules, peptides, peptide mimetics, small molecules, organic molecules, inorganic molecules, proteins, hormones, transcription factors, calcium blockers, variants of calcium blockers, or the like) can be contacted to a cell and an effect on Orai or Stim polypeptides or complexes and/or orai or stim gene activity and/or expression monitored by any of the assays described herein or known in the art.

Furthermore, expression of Orai or Stim can be detected, e.g., via northern analysis or quantitative (e.g., real time) RT-PCR, before and after application of potential expression modulators. Similarly, promoter regions of orai and/or stim gene(s) of interest (e.g., generally sequences in the region of the start site of transcription, e.g., within 5 KB of the start site, e.g., 1 KB, or less e.g., within 500 BP or 250 BP or 100 BP of the start site) can be coupled to reporter constructs (CAT, beta-galactosidase, luciferase or any other available reporter) and can be similarly be tested for expression activity modulation by the potential modulator. In either case, the assays can be performed in a high-throughput fashion, e.g., using automated fluid handling and/or detection systems, in a serial or parallel fashion. Similarly, activity modulators can be tested by contacting a potential modulator to an appropriate cell using any of the activity detection methods herein, regardless of whether the activity that is detected is the result of activity modulation, expression modulation or both.

In any of the assays herein, control compounds can be administered and the activity of the control compounds compared to those of the test compounds to verify that changes in activity resulting from application of the test compound are not artifacts. For example, control compounds can include the various dyes, buffers, adjuvants, carriers, or the like that the test compounds are typically administered with, but lacking a putative test compound.

Example: Genome Wide Screen

The present invention includes a genome-wide screen, based upon direct Ca²⁺ influx measurements, that identifies genes that are required for CRAC channel activity. Agonist-receptor interactions at the plasma membrane often lead to the generation of inositol 1,4,5-trisphosphate (IP₃) in many cell types, which in turn releases Ca²⁺ from internal stores in the endoplasmic reticulum. Such a depletion of Ca²⁺ stores activates Ca²⁺ permeable store-operated ion channels (SOCs) in the plasma membrane, allowing a sustained Ca²⁺ influx termed capacitative or stored-operated Ca²⁺ entry. In certain cell types, depletion of internal Ca²⁺ stores by either antigen/agonist binding or sarcoplasmic/endoplasmic reticulum Ca²⁺ (SERCA) pump inhibitors (e.g., thapsigagin (TG)), or dialysis of the cytosol by a whole-cell pipette solution during patch-clamp recordings activate a highly Ca²⁺-selective ion channel termed the Ca²⁺ release-activated Ca²⁺ (CRAC) channel, which has an extremely low unitary conductance for Ca²⁺ (24 fs).

The single transmembrane-spanning Ca²⁺-binding protein, STIM1, is necessary in the coupling process of SOCs to store depletion, and is proposed to function as an ER Ca²⁺ sensor to provide the trigger for SOC activation. Accordingly, this invention has validated stim and has identified olf186-F (Orai) as essential for Ca²⁺ signaling and activation of CRAC current, which also confirms a recent report. In addition, evidence based upon overexpression and mutation analysis is provided that Orai forms an essential part of the CRAC channel (e.g., the calcium pore).

In mammalian cells, overexpression of STIM1 increases Ca²⁺ influx rates and CRAC currents about two-fold, but in S2 cells, overexpression of Stim alone does not increase CRAC current, which is consistent with Stim serving as a channel activator, rather than the channel itself. In contrast, transfection of olf186-F by itself increased CRAC current densities three-fold; and co-transection of olf186-F with Stim resulted in an eight-fold enhancement and the largest CRAC currents ever recorded. These results support the hypothesis that olf186-F constitutes part of the CRAC channel and that Stim serves as the messenger for its activation. Consistent with this hypothesis, the CRAC channel activation kinetics during passive Ca²⁺ store depletion were significantly faster with co-transfected Stim. Many details of the mechanism of CRAC channel activation remain to be clarified including some of the protein-protein interactions that underlie trafficking and channel activation. Site-directed mutagenesis in a heterologous expression system is also used in the examples below to define the putative pore-forming region within olf186-F.

Similar to Stim, knockdown of olf186-F did not produce a severe cell growth phenotype. It was neither a hit in a previous screen of cell survival nor in any other published Drosophila whole-genome RNAi screen. The olf186-F gene is a member of a highly conserved gene family that contains three homologs in mammals, two in chicken, three in zebrafish, and one member only in fly and worm (FIG. 8A). C09F5.2, the only homolog in C. elegans, is expressed in intestine, hypodermis, reproductive system as well as some neuron-like cells in the head and tail regions (http://wormbase.org). Worms under RNAi treatment against C09F5.2 are sterile.

RNAi (RNA interference) is a phenomenon in which the introduction of double-stranded RNA (dsRNA) into a diverse range of organisms and cell types causes degradation of the complementary mRNA. In the cell, long dsRNAs are cleaved into short (e.g., 21-25 nucleotide) small interfering RNAs (siRNAs), by a ribonuclease. The siRNAs subsequently assemble with protein components into an RNA-induced silencing complex (RISC), unwinding in the process. The activated RISC then binds to complementary transcripts by base pairing interactions between the siRNA antisense strand and the mRNA. The bound mRNA is then cleaved and sequence specific degradation of mRNA results in gene silencing. The RNAi machinery appears to have evolved to protect the genome from endogenous transposable elements and from viral infections. Thus, RNAi can be induced by introducing nucleic acid molecules complementary to the target mRNA to be degraded.

Accordingly, in one embodiment, the invention provides methods of identifying genes that inhibit Ca²⁺ influx upon store emptying by TG. The screen in this study made use of the ability of thapsigargin (TG) to send NFAT-GFP to the nucleus in S2 cells, providing an assay for disruption of signaling anywhere in the cascade from elevated intracellular concentrations of Ca²⁺ ([Ca²⁺]ᵢ) to calcineurin activation and nuclear relocalization of NFAT (nuclear factor of activated T cells). The fly gene olf186-F (named Orai) was identified in the screen, and a human homolog on chromosome 12 was shown to be mutated in SCID patients, resulting in the loss of CRAC channel activity. Heterologous expression of the wild-type human homolog, named Orai 1, restored CRAC channel activity in SCID T cell lines.

Additional Details Regarding Transmembrane Potential Measurements, Ca⁺⁺ Indicator Dyes and Transmembrane Dyes

As noted above, the invention optionally includes monitoring calcium flux, e.g., directly using indicator dyes, or indirectly by monitoring transmembrane potential (TM potential), e.g., to track CRAC channel activity (e.g., activity of Orai and/or Stim).

Ca⁺⁺ Indicator Dyes and Radioactive Tracer Measurements

In addition to various potentiometric probe dyes for monitoring changes in TMP, any of a variety of Ca⁺⁺ indicator dyes can also be used to directly monitor Ca⁺⁺ levels (whether in the cell, in organelles, outside of the cell, in cell lysates, or the like). This approach represents a preferred approach to monitoring Ca⁺⁺ levels.

Fluorescent indicator probes that display a change in a fluorescence upon binding Ca²⁺ have been used to investigate changes in intracellular Ca²⁺ concentrations using fluorescence microscopy, flow cytometry and fluorescence spectroscopy. See, e.g., Lipp et al. (2001) “Photometry, video imaging, confocal and multi-photon microscopy approaches in calcium signalling studies.” in Cellular Calcium Practical Approach, 2nd Ed., Tepikin A, Ed. pp. 17-44 (2001); Burchiel et al. (2000) “Analysis of free intracellular calcium by flow cytometry: multiparameter and pharmacologic applications” Methods 21:221-230; Haugland and Johnson (1999) “Intracellular Ion Indicators.” Fluorescent and Luminescent Probes for Biological Activity, 2nd Ed., Mason W T, Ed., pp. 40-50; Takahashi et al. (1999) “Measurement of intracellular calcium.” Physiol Rev 79:1089-1125; Silver (1998) “Ratio imaging: practical considerations for measuring intracellular calcium and pH in living tissue.” Methods Cell Biol 56:237-251; Kao (1994) “Practical aspects of measuring [Ca2+] with fluorescent indicators.” Kao Methods Cell Biol 40:155-181; Haughland (2002) Molecular Probes Handbook of Fluorescent Probes and Research Products, Ninth Edition and the references cited therein.

For example, visible light-excitable and UV-excitable Ca⁺⁺ indicators are available. Visible light indicators provide one convenient type of Ca⁺⁺ indicator, in that they can be used with typical laser instrumentation (e.g., a laser scanning microscope), or even standard light microscopy. In addition, these dyes display large increases in fluorescence intensity upon mobilization of Ca⁺⁺, resulting in easily detectable changes in Ca⁺⁺ concentration and flux.

A variety of Ca⁺⁺ indicator dyes are available, including: Bis-fura, Calcium Green-1, Calcium Green-2, Calcium Green-5N, Calcium Orange, Calcium Crimson, Fluo-3, Fluo-4, Fluo-5F, Fluo-4FF, Fluo-5N, Fura-2, Fura-4F, Fura-5F, Fura-6F, Fura-FF, Fura Red, Indo-1, Indo-5F, Mag-fluo-4, Mag-fura-2, Mag-fura-5, Mag-indo-1, Magnesium Green, Oregon Green 488 BAPTA-1, Oregon Green 488 BAPTA-2, Oregon Green 488 BAPTA-6F, Oregon Green 488 BAPTA-5N, Quin-2, Rhod-2, Rhod-FF Rhod-5N, X-rhod-1, X-rhod-5F and X-rhod-FF. See also, probes(dot)invitrogen(dot)com/handbook/tables/0355(dot)html “Table 19.1—Summary of fluorescent Ca2+ indicators available from Molecular Probes.”

One useful class of Ca⁺⁺ indicators are the Fluo-3, Fluo-4, Rhod-3 and related derivatives. Fluo-3 imaging has been widely used to study many elementary processes in Ca⁺⁺ signaling. Other useful dyes include, e.g., Fura-4F, fura-5F, fura-6F and fura-FF which display increased sensitivity to intracellular Ca²⁺ concentration, e.g., in the 0.5-5 μM range, as compared with fura-2. The fluo-3, fluo-4, Oregon Green 488 BAPTA, Calcium Green, X-rhod-1 and Fura Red indicators and their variants allow Ca⁺⁺ detection over a wide concentration range and offer increased brightness and reduced phototoxicity. See e.g., probes(dot)invitrogen(dot)com/handbook/sections/1901(dot)html.

Loading and calibration of calcium dyes can be carried out using available techniques. For example, the AM ester technique is one typical method for loading fluorescent ion indicators. In this technique, carboxylate groups of indicators for Ca²⁺ are derivatized, rendering the indicator permeant to membranes. Once inside the cell, these derivatized indicators are hydrolyzed by intracellular esterases, releasing the indicator. Calibration procedures can include recording fluorescence signals at a series of precisely manipulated Ca⁺⁺ concentrations. The resulting titration curve can be linearized with a Hill plot or analyzed directly by nonlinear regression to yield a K_{d}. For in vitro calibrations of Ca⁺⁺ indicators, EGTA buffering produces defined Ca⁺⁺ concentrations that are calculated from the Kd of the Ca⁺⁺-EGTA complex. See, e.g., probes(dot)invitrogen(dot)com/handbook/boxes/0428(dot)html “Note 19.1—Technical Focus: Loading and Calibration of Intracellular Ion Indicators.” See also Lipp et al. “Photometry, video imaging, confocal and multi-photon microscopy approaches in calcium signalling studies.” Cellular Calcium Practical Approach, 2nd Ed., Tepikin A, Ed. pp. 17-44 (2001); O'Malley et al. (1999) “Fluorescent calcium indicators: subcellular behavior and use in confocal imaging.” Methods Mol Biol 122:261-303; Takahashi (1999) “Measurement of intracellular calcium.” Physiol Rev 79:1089-1125; Scheenen et al. (1998) “Intracellular Measurement of Calcium Using Fluorescent Probes.” Cell Biology: A Laboratory Handbook, 2nd Ed., Vol. 3, Celis J E, Ed. pp. 363-374; Silver (1998) “Ratio imaging: practical considerations for measuring intracellular calcium and pH in living tissue.” Cell Biol 56:237-251; Kao (1994) “Practical aspects of measuring [Ca⁺⁺ ] with fluorescent indicators.” Methods Cell Biol 40: 155-181 (1994); Negulescu (1990) “Intracellular ion activities and membrane transport in parietal cells measured with fluorescent dyes.” Methods Enzymol 192:38-81; Tsien and Pozzan (1989) “Measurement of cytosolic free Ca⁺⁺ with quin2.” T. Methods Enzymol 172: 230-262.

In addition to monitoring Ca⁺⁺ changes using fluorescent indicators, it is also possible to rapidly increase or reduce intracellular Ca⁺⁺ concentration and monitor resulting effects. For example, the photolabile chelator, o-nitrophenyl EGTA exhibits a high selectivity for Ca⁺⁺, a 12,500-fold decrease in affinity for Ca⁺⁺ upon UV illumination and a high photochemical quantum yield. DM-Nitrophen is another example of a similar reagent. See also, Zimmermann et al. (1995) “Kinetics of prephosphorylation reactions and myosin light chain phosphorylation in smooth muscle. Flash photolysis studies with caged calcium and caged ATP.” J Biol Chem 270: 23966-23974; Valdivia et al. (1995) “Rapid adaptation of cardiac ryanodine receptors: modulation by Mg²⁺ and phosphorylation.” Science 267:1997-2000; Ellis-Davies et al. (1994) “Nitrophenyl-EGTA, a photolabile chelator that selectively binds Ca⁺⁺ with high affinity and releases it rapidly upon photolysis.” Proc Natl Acad Sci USA 91:187-191.

As an alternative to dye measurements, radioactive elemental forms of calcium (e.g., ⁴¹Ca, ⁴⁵Ca, etc.) can be used to trace flow of calcium into and out of the cell, by monitoring flow of radioactive Ca⁺⁺. See, e.g., Southon et al. (1994) “⁴¹Ca as a tracer for calcium uptake and deposition in heart tissue during ischemia and reperfusion” Nuclear Instruments and Methods in Physics Research Section B, Volume 92, Issue 1-4:489-491; and Abercrombie et al. (1981) “Uptake and release of ⁴⁵Ca by Myxicola axoplasm” The Journal of General Physiology, 78:413-429.

TMP Measurements

In general, the distribution of a permeable ion between the inside and outside of a cell or other membrane depends on the transmembrane potential of the cell membrane. In particular, for ions separated by a semi-permeable membrane, the electrochemical potential difference (ΔΞⱼ) which exists across the membrane, is given by Δμⱼ=2.3 RT log [j_{I})/jₒ]+ZE_{R}F, where R is the universal gas constant, T is an absolute temperature of the composition, F is Faraday's constant in coulombs, [j_{I}] is the concentration of an ion (j) on an internal or intracellular side of the at least one membrane, [jₒ] is the concentration of j on an external or extracellular side of the at least one membrane, z is a valence of j and E_{R} is a measured transmembrane potential. Thus, the calculated equilibrium potential difference (Eⱼ) for ion j=−2.3RT(zF)⁻¹ log [j_{I}]/[jₒ] (this is often referred to as the “Nernst equation”). See, Selkurt, ed. (1984) Physiology 5^{th} Edition, Chapters 1 and 2, Little, Brown, Boston, Mass. (ISBN 0-316-78038-3); Stryer (1995) Biochemistry 4^{th} edition Chapters 11 and 12, W.H. Freeman and Company, NY (ISBN 0-7167-2009-4); Haugland (1996) Handbook of Fluorescent Probes and Research Chemicals Sixth Edition by Molecular Probes, Inc. (Eugene Oreg.) Chapter 25 (Molecular Probes, 1996) and http://www.probes.com/handbook/sections/2300.html (Chapter 23 of the on-line 1999 version of the Handbook of Fluorescent Probes and Research Chemicals Sixth Edition by Molecular Probes, Inc.) (Molecular Probes, 1999) and Hille (1992) Ionic Channels of Excitable Membranes, second edition, Sinauer Associates Inc. Sunderland, Mass. (ISBN 0-87893-323-9) (Hille), for an introduction to transmembrane potential and the application of the Nernst equation to transmembrane potential. In addition to the Nernst equation, various calculations which factor in the membrane permeability of an ion, as well as Ohm's law, can be used to further refine the model of transmembrane potential difference, such as the “Goldman” or “constant field” equation and Gibbs-Donnan equilibrium. See Selkurt, ed. (1984) Physiology 5^{th} Edition, Chapter 1, Little, Brown, Boston, Mass. (ISBN 0-316-78038-3) and Hille at e.g., chapters 10-13.

Increases and decreases in resting transmembrane potential—referred to as membrane depolarization and hyperpolarization, respectively—play a central role in many physiological processes, including ion-channel gating. Potentiometric optical probes (typically potentiometric dyes) provide a tool for measuring transmembrane potential and changes in transmembrane potential over time (e.g., transmembrane potential responses following the addition of a composition which affects transmembrane potential) in membrane containing structures such as organelles, cells and in vitro membrane preparations. In conjunction with probe imaging techniques (e.g., visualization of the relevant dyes), dye probes are used to map variations in transmembrane potential across cells membranes.

Potentiometric probes include cationic or zwitterionic styryl dyes, cationic rhodamines, anionic oxonols, hybrid oxonols and merocyanine 540. The class of dye determines factors such as accumulation in cells, response mechanism and cell toxicity. See, Molecular Probes 1999 and the reference cited therein; Plasek et al. (1996) “Indicators of Transmembrane potential: a Survey of Different Approaches to Probe Response Analysis.” J Photochem Photobiol; Loew (1994) “Characterization of Potentiometric Membrane Dyes.” Adv Chem Ser 235, 151 (1994); Wu and Cohen (1993) “Fast Multisite Optical Measurement of Transmembrane potential” Fluorescent and Luminescent Probes for Biological Activity, Mason, Ed., pp. 389-404; Loew (1993) “Potentiometric Membrane Dyes.” Fluorescent and Luminescent Probes for Biological Activity, Mason, Ed., pp. 150-160; Smith (1990) “Potential-Sensitive Molecular Probes in Membranes of Bioenergetic Relevance.” Biochim Biophys Acta 1016, 1; Gross and Loew (1989) “Fluorescent Indicators of Transmembrane potential: Microspectrofluorometry and Imaging.” Meth Cell Biol 30, 193; Freedman and Novak (1989) “Optical Measurement of Transmembrane potential in Cells, Organelles, and Vesicles” Meth Enzymol 172, 102 (1989); Wilson and Chused (1985) “Lymphocyte Transmembrane potential and Ca⁺²-Sensitive Potassium Channels Described by Oxonol Dye Fluorescence Measurements” Journal of Cellular Physiology 125:72-81; Epps et al. (1993) “Characterization of the Steady State and Dynamic Fluorescence Properties of the Potential Sensitive dye bis-(1,3-dibutylbarbituric acid) trimethine oxonol (DiBAC₄(3) in model systems and cells” Chemistry of Physics and Lipids 69:137-150, and Tanner et al. (1993) “Flow Cytometric Analysis of Altered Mononuclear Cell Transmembrane potential Induced by Cyclosporin” Cytometry 14:59-69.

Potentiometric dyes are typically divided into at least two categories based on their response mechanism. The first class of dyes, referred to as fast-response dyes (e.g., styrylpyridinium dyes; see, e.g., Molecular Probes (1999) at Section 23.2), operate by a change in the electronic structure of the dye, and consequently the fluorescence properties of the dye, i.e., in response to a change in an electric field which surrounds the dye. Optical response of these dyes is sufficiently fast to detect transient (millisecond) potential changes in excitable cells, e.g., isolated neurons, cardiac cells, and even intact brains. The magnitude of the potential-dependent fluorescence change is often small; fast-response probes typically show a 2-10% fluorescence change per 100 mV.

The second class of dyes, referred to as slow-response (or “Nernstian”) dyes (See, e.g., Molecular Probes, 1999 at Section 23.3), exhibit potential-dependent changes in membrane distribution that are accompanied by a fluorescence change. The magnitude of their optical responses is typically larger than that of fast-response probes. Slow-response probes, which include cationic carbocyanines, rhodamines and anionic oxonols, are suitable for detecting changes in a variety of transmembrane potentials of, e.g., nonexcitable cells caused by a variety of biological phenomena, including ion channel permeability. The structures of a variety of available slow response dyes are found e.g., at table 25.3 of Molecular Probes (1996).

Many slow, Nernstian dyes such as carbocyanines, rhodamines and oxonols are used to measure transmembrane potential by virtue of voltage-dependent dye redistribution and fluorescence changes resulting from the redistribution. Fluorescence changes which may be caused by redistribution include: a change of the concentration of the fluorophore within the cell or vesicle, a change in the dye fluorescence due to aggregation or a change in dye fluorescence due to binding to intracellular or intravesicular sites. Typically, 10-15 minutes of equilibration time is used to allow the dyes to redistribute across the cell membrane after changing the transmembrane potential.

Examples of available anionic dyes that can be used for measurement of transmembrane potential include the anionic bis-isoxazolone oxonols which accumulate in the cytoplasm of depolarized cells by a Nernst equilibrium-dependent uptake from the extracellular solution. Of the oxonols studied in one reference (“Kinetics of the Potential-Sensitive Extrinsic Probe Oxonol VI in Beef Heart Submitochondrial Particles.” J. C. Smith, B. Chance. J Membrane Biol 46, 255 (1979)), oxonol VI gave the largest spectral shifts, with an isosbestic point at 603 nm. Oxonol VI responds to changes in potential more rapidly than oxonol V.

The three common bis-barbituric acid oxonols, often referred to as DiBAC dyes, form a family of spectrally distinct potentiometric probes with excitation maxima at approximately 490 nm (DiBAC₄(3), 530 nm (DiSBAC₂(3)) and 590 nm (DiBAC₄(5)). DiBAC₄(3) has been used in many publications that cite using a “bis-oxonol” (Molecular Probes, 1999, chapter 23). The dyes enter depolarized cells where they bind to intracellular proteins or membranes and exhibit enhanced fluorescence and red spectral shifts. Increased depolarization results in more influx of the anionic dye and thus an increase in fluorescence. DiBAC₄(3) has particularly high voltage sensitivity. The long-wavelength DiSBAC₂(3) has frequently been used in combination with the UV light-excitable Ca²⁺ indicators indo-1 or fura-2 for the simultaneous measurements of transmembrane potential and Ca²⁺ concentrations (id. at Table 23.2).

Classes of cationic membrane permeable dyes that can be used as ion sensing compositions include, e.g., indo-carbocyanine dyes, thio-carbocyanine dyes, oxa-carbocyanine dyes (see Molecular Probes on-line catalogue, updated as of Aug. 10, 2000, at section 23.3, entitled “Slow-Response Dyes;” http://www.probes.com/handbook/sections/2303.html). See also, Sims, et al. (1974) “Studies on the Mechanism by Which Cyanine Dyes Measure Membrane Potential in Red Blood Cells and Phosphatidylcholine Vesicles,” Biochemistry 13, 3315; Cabrini and Verkman (1986) “Potential-Sensitive Response Mechanism of DiS-C3(5) in Biological Membranes,” Membrane Biol 92, 171; Guillet and Kimmich (1981) “DiO-C3-(5) and DiS-C3-(5): Interactions with RBC, Ghosts and Phospholipid Vesicles,” J Membrane Biol 59, 1; Rottenberg and Wu (1998) “Quantitative Assay by Flow Cytometry of the Mitochondrial Membrane Potential in Intact Cells,” Biochim Biophys Acta 1404, 393 (1998).

Additional Details Regarding Assay Formats

In general, changes in the level of fluorescence of the biological sample (e.g., containing Orai and/or Stim and/or coding nucleic acids thereof)-test compound mixture are detected, where a change in fluorescence is indicative of calcium concentration and/or a change in transmembrane potential (e.g., by using a dye as noted above). Typically, the assay methods described herein are used to detect the effect of the test compound on the calcium concentration and/or transmembrane potential of a cell or other membrane. Where one is seeking to determine the effect of a test compound on a cell's calcium concentration and/or transmembrane potential, e.g., through a change in ion flux, transport, membrane permeability, or the like, one can expose the cell, membrane, etc., to the test compound and the cell, etc., is examined for the presence of a previously absent fluorescent signal (or the absence of a previously present fluorescent signal).

For example, in one assay format, a dye is contacted to a biological sample. In accordance with these methods, the sample can be placed into a reaction vessel, such as a microwell dish, and the level of fluorescence from the composition is measured, optionally over a period of time. This can be used to provide an initial or background level of fluorescence indicative of an existing calcium concentration and/or transmembrane potential for the biological sample. A selected test compound is then added to the biological sample (or these procedures are carried out in parallel, providing control and experimental samples). The test compound can be tested alone, or is added before, together or after addition of putative modulator to determine its effect on CRAC responses (e.g. enhancement or inhibition). Following the stimulus, the fluorescence level of the biological sample is again measured (typically over time) and compared to the initial fluorescent level or the fluorescence level in a control cell population (e.g., which is exposed to a control calcium concentration and/or TMP modulator, e.g., a Ca⁺⁺ flux modulator). Any change in the level of fluorescence not attributable to dilution by the test compound (as determined from an appropriate control) is then attributable to the effect the test compound has on the cell's transmembrane potential, or rate of calcium concentration and/or TMP change in response to depolarization or hyperpolarization events.

A suitable negative control can be used in the assay, such as a biological sample that does not include the Orai and/or Stim and/or a coding nucleic acid, to ensure that the effect being observed is caused by the relevant protein or complex. Similarly, a suitable positive control can be used in the assay, such as a test compound known to effect the protein, gene or complex under study, to ensure that the biological sample components are suitably active.

These types of reactions are carried out in an appropriate reaction receptacle that allows measurement of fluorescence, in situ. As such, the receptacle is typically a transparent reaction vessel, such as a test tube, cuvette, a reaction well in a multiwell plate, or a transparent conduit, e.g., a capillary, microchannel or tube.

The assay methods of the present invention are particularly useful in performing high-throughput (greater than 1,000 compounds/day) and even ultra-high throughput (e.g., greater than 10,000 compounds/day) screening of chemical libraries, e.g., in searching for modulator leads. These experiments may be carried out in parallel by a providing a large number of reaction mixtures (e.g., cell suspensions as described herein) in separate receptacles, typically in a multiwell format, e.g., 96 well, 324 well or 1536 well plates. Different test compounds (library members) are added to separate wells, and the effect of the compound on the reaction mixture is ascertained, e.g., via the fluorescent signal. These parallelized assays are generally carried out using specialized equipment e.g., as described above to enable simultaneous processing of large numbers of samples, i.e., fluid handling by robotic pipettor systems and fluorescent detection by multiplexed fluorescent multi-well plate readers.

Patch Clamping

As noted above, monitoring of transmembrane dye flow is a preferred method of monitoring test compound effects on ion channels. A second preferred method uses voltage clamping, such as patch clamping. This is a particularly useful method e.g., when using Xenopus oocytes or cells in culture.

A voltage clamp allows for the measurement of ion currents flowing across a cell membrane. Originally, the voltage clamp used two electrodes and a feedback circuit for transmembrane measurements. In the original Cole-Marmount voltage clamp, both electrodes are placed inside a cell and transmembrane voltage is recorded through one of the electrodes (the “voltage electrode”) relative to an outside reference (e.g., ground). The second electrode passes current into the cell and is termed the “current electrode”.

Briefly, a “holding voltage” is maintained across the cell membrane. Anytime the cell makes a deviation from this holding voltage by passing an ion current across its membrane, an operational amplifier generates an “error signal”. The error signal is the difference between the holding voltage specified by the experimenter and the actual voltage of the cell. The feedback circuit of the voltage clamp passes current into the cell (via the current electrode) in the polarity needed to reduce the error signal to zero. Thus, the current is applied in a polarity opposite current that the cell is passing across its membrane, and the clamp circuit provides a current that is the mirror image of the cellular current. This mirror or “clamp current” can be easily measured, giving an accurate reproduction of the currents flowing across the cell's membrane (although in the opposite polarity).

A modern variant of this general method is the “patch clamp” which uses a single electrode device. The patch clamp technique is in common use to monitor the flow of ions across a membrane (Neher E (1992) “Nobel lecture. Ion channels for communication between and within cells” Neuron. 8(4):605-12). The patch clamp technique involves applying a very finely drawn glass micropipette onto the surface of a cell to form an electrode. This electrode is pressed against a cell membrane and suction is applied to the inside of the electrode to pull the cell's membrane inside the tip of the electrode. This suction causes the cell to form a tight seal with the electrode (a “giga-ohm seal,” as the electrical resistance of the seal is in excess of one giga-ohm). From this point, at least 4 different experimental approaches can be taken. First, the electrode can be left sealed to a patch of membrane (a “cell-attached patch”). This allows for the recording of currents through single ion channels in that patch of membrane. Second, the electrode can be withdrawn from the cell, ripping a patch of membrane off of the cell. This forms an “inside-out” patch. This is useful when the environment on the inside of an ion channel is to be studied. Third, the electrode can be withdrawn from the cell, allowing a blob of membrane to bud from the cell. When the electrode is pulled away, this blob will part from the cell and reform as a ball of membrane on the end of the electrode, with the outside of the membrane being the surface of the ball (thus the name “outside out patch”). Such “outside out” patching permits examination of the properties of an ion channel when it is protected from the outside environment, but not in contact with it's usual environment. Fourth, the electrode can be left in place, but harder suction is applied to rupture the portion of the cell's membrane that is inside the electrode, providing access to the intracellular space of the cell. This is known as “whole-cell recording”. This method is also sometimes misnamed a “whole cell patch.” The advantage of whole cell recording is that the sum total current that flows across the cell's membrane can be recorded.

Thus, the voltage clamping such as the patch clamp technique allows the recording of single ion-channel currents, or alternatively currents from entire small cells. In the context of the present invention, this provides a platform for the analysis of changes in currents that result from application of a test compound of modulator.

A modern variant of the classical patch clamp that can be adapted to the present invention is the planar patch clamp, which uses a planar array of PDMS electrodes that mimic a classical glass electrode (Klemic et al. (2002) “Micromolded PDMS Electrode Allows Patch Clamp Electrical Recording From Cells” Biosensors and Bioelectronics 597-604). This modern patch clamp is suited to high throughput patch clamp analysis, allowing many different cells to be analyzed for ion channel activity simultaneously.

Patch clamp devices are also commercially available, e.g., from Axon Instruments.

Additional Screening System Details

Automated systems of the invention can facilitate the screening methods noted above (both in vitro and in vivo screening methods). That is, systems that facilitate cell or biochemical sample based screening for Orai/orai and/or Stim/stim expression and/or activity are a feature of the invention. Similarly, systems designed to monitor physiological responses of animals, including non-human transgenic laboratory animals, are also a feature of the invention. System features herein are generally applicable to the methods herein and vice-versa.

Biological/Biochemical Sources/Libraries

High-throughput automated systems that detect compounds that bind to and/or modulate an activity of a Orai or Stim polypeptide, or complex thereof, typically include a biological/biochemical sample (which includes the polypeptide or complex, e.g., any cell or other material described herein) and a source of a plurality of test compounds. A detector detects binding of one or more of the test compounds to the Orai or Stim polypeptide, or modulation of a level or activity of the polypeptide or complex (or mRNA transcript(s) corresponding to the polypeptide or polypeptide complex) by the test compounds, thereby identifying a putative modulator, acid receptor binding moiety, etc., that binds to or otherwise modulates an activity of the Orai or Stim polypeptide or complex.

The source of test compound for such systems and in the practice of the methods of the invention can be any commercially available or proprietary library of materials, including compound libraries from Sigma (St. Louis Mo.), Aldrich (St. Louis Mo.), Agilent Technologies (Palo Alto, Calif.) or the like.

The format of the library will vary depending on the system to be used. In one typical embodiment, libraries of sample materials are arrayed in microwell plates (e.g., 96, 384 or more well plates), which can be accessed by standard fluid handling robotics, e.g., using a pipettor or other fluid handler with a standard ORCA robot (Optimized Robot for Chemical Analysis) available from Beckman Coulter (Fullerton, Calif.). Standard commercially available workstations such as the Caliper Life Sciences (Hopkinton, Mass.) Sciclone ALH 3000 workstation and Rapidplate™ 96/384 workstation provide precise 96 and 384-well fluid transfers in a small, highly scalable format. Plate management systems such as the Caliper Life Sciences Twister® II Advanced Capability Microplate Handler for End-Users, OEM's and Integrators provide plate handling, storage and management capabilities for fluid handling, while the Presto™ AutoStack provides fast reliable access to consumables presenting trays of tips, reagents, microplates or deep wells to an automated device (e.g., the ALH 3000) without robotic arm intervention.

Microfluidic systems for handling and analyzing microscale fluid samples, including cell based and non-cell based approaches that can be used for analysis of test compounds on biological samples in the present invention are also available, e.g., the Caliper Life Sciences various LabChip® technologies (e.g., LabChip® 90 and 3000) and Agilent Technologies (Palo Alto, Calif.) 2100 and 5100 devices. Similarly, interface devices between microfluidic and standard plate handling technologies are also commercially available. For example, the Caliper Technologies LabChip® 3000 uses “sipper chips” as a “chip-to-world” interface that allows automated sampling from microtiter plates. To meet the needs of high-throughput environments, the LabChip® 3000 employs four or even twelve sippers on a single chip so that samples can be processed, in parallel, up to twelve at a time. Solid phase libraries of materials can also be conveniently accessed using sipper or pipetting technology, e.g., solid phase libraries can be gridded on a surface and dried for later rehydration with a sipper or pipette and accessed through the sipper or pipette.

As already noted, with regard to the systems and methods of the invention, the particular libraries of compounds can be any of those that now exist, e.g., those that are commercially available, or that are proprietary. A number of libraries of test compounds exist, e.g., those from Sigma (St. Louis Mo.), and Aldrich (St. Louis Mo.). Other current compound library providers include Actimol (Newark Del.), providing e.g., the Actiprobe 10 and Actiprobe 25 libraries of 10,000 and 25,000 compounds, respectively; BioMol (Philadelphia, Pa.), providing a variety of libraries, including natural compound libraries and the Screen-Well™ Ion Channel ligand library which are usefully screened against the receptors herein, as well as several other application specific libraries; Enamine (Kiev, Ukraine) which produces custom libraries of billions of compounds from thousands of different building blocks, TimTec (Newark Del.), which produces general screening stock compound libraries containing >100,000 compounds, as well as template-based libraries with common heterocyclic lattices, libraries for targeted mechanism based selections, including kinase modulators, GPCR Ligands, channel modulators, etc., privileged structure libraries that include compounds containing chemical motifs that are more frequently associated with higher biological activity than other structures, diversity libraries that include compounds pre-selected from available stocks of compounds with maximum chemical diversity, plant extract libraries, natural products and natural product-derived libraries, etc; AnalytiCon Discovery (Germany) including NatDiverse (natural product analogue screening compounds) and MEGAbolite (natural product screening compounds); Chembridge (San Diego, Calif.) including a wide array of targeted or general and custom or stock libraries; ChemDiv (San Diego, Calif.) providing a variety of compound diversity libraries including CombiLab and the International Diversity Collection; Comgenix (Hungary) including ActiVerse™ libraries; MicroSource (Gaylordsville, Conn.) including natural libraries, agro libraries, the NINDS custom library, the genesis plus library and others; Polyphor (Switzerland) including privileged core structures as well as novel scaffolds; Prestwick Chemical (Washington D.C.), including the Prestwick chemical collection and others that are pre-screened for biotolerance; Tripos (St. Louis, Mo.), including large lead screening libraries; and many others. Academic institutions such as the Zelinsky Institute of Organic Chemistry (Russian Federation) also provide libraries of considerable structural diversity that can be screened in the methods of the invention. Pyrazole libraries are one useful library for screening in the present invention (see, Mol. Pharmacol. 2006 April; 69(4):1413-20).

Detectors and Other System Components

Although the devices and systems specifically illustrated herein are generally described in terms of the performance of a few or one particular operation, it will be readily appreciated from this disclosure that these systems permit easy integration of additional operations. For example, the systems described will optionally include structures, reagents and systems for performing virtually any number of operations both upstream and downstream from the operations specifically described herein. Such upstream operations include sample handling and preparation operations, e.g., cell separation, extraction, purification, culture, amplification, cellular activation, labeling reactions, dilution, aliquotting, and the like. Similarly, downstream operations may include similar operations, including, e.g., separation of sample components, labeling of components, assays and detection operations, movement of components into contact with cells or other membrane preparations, or materials released from cells or membrane preparations, or the like.

Upstream and downstream assay and detection operations include, without limitation, cell fluorescence assays, cell activity assays, receptor/ligand assays, immunoassays, and the like. Any of these elements can be fixed incorporated into the systems herein.

Instrumentation for high throughput optical screening of cell assays is available. In addition to the systems noted herein, other automated approaches can also be practiced with the dyes and methods of the invention. For example, the FLIPR (Fluorescence Imaging Plate Reader) was developed to perform quantitative optical screening for cell based kinetic assays (Schroder and Neagle (1996) “FLIPR: A New Instrument for Accurate, High Throughput Optical Screening” Journal of Biomolecular Screening 1(2):75-80). This device can be adapted to the present invention, e.g., by using dyes to monitor TMP and/or calcium concentration or capacitance, as discussed herein.

In general in the present invention, materials such as cells and dyes are optionally monitored and/or detected so that an activity such as TMP activity can be determined. Depending on the label signal measurements, decisions can be made regarding subsequent operations, e.g., whether to assay a particular modulator in detail to determine detailed receptor binding/activity kinetic information.

The systems described herein generally include fluid handling devices, as described above, in conjunction with additional instrumentation for controlling fluid transport, flow rate and direction within the devices, detection instrumentation for detecting or sensing results of the operations performed by the system, processors, e.g., computers, for instructing the controlling instrumentation in accordance with preprogrammed instructions, receiving data from the detection instrumentation, and for analyzing, storing and interpreting the data, and providing the data and interpretations in a readily accessible reporting format. Patch clamps, or other features described herein are also optionally features of the invention.

Controllers

A variety of controlling instrumentation is optionally utilized in conjunction with the fluid handling elements described above, for controlling the transport and direction of fluids and/or materials (biological samples, test compounds, etc.) within the systems of the present invention. Controllers typically include appropriate software directing fluid and material transport in response to user instructions.

Typically, the controller systems are appropriately configured to receive or interface with a fluid handling or other system element as described herein. For example, the controller and/or detector, optionally includes a stage upon which a sample is mounted to facilitate appropriate interfacing between the controller and/or detector and the rest of the system. Typically, the stage includes an appropriate mounting/alignment structural elements, such as a nesting well, alignment pins and/or holes, asymmetric edge structures (e.g., to facilitate proper alignment of slides, microwell plates or microfluidic “chips”), and the like.

Detectors

Within the systems, detectors can take any of a variety of forms. The various fluid handling stations noted above often come with integrated detectors, e.g., optical or fluorescent detectors. However, other detectors such as patch clamp devices, fluorescence detectors that detects FRET, changes in membrane potential or flow of a dye into or out of the cell are also suitable, depending on the application.

Generally, devices herein optionally include signal detectors, e.g., which detect fluorescence, phosphorescence, radioactivity, pH, charge, absorbance, luminescence, temperature, magnetism or the like. As noted, fluorescent and patch clamp detection is especially preferred and generally used for detection of voltage changes, or flow of voltage sensitive compounds (however, as noted, upstream and downstream operations can be performed on cells, dyes, modulators or the like, which can involve other detection methods).

System signal detectors are typically disposed adjacent to a site of reaction or mixing between the biological/biochemical sample and the test compound. This site can be a test tube, microwell plate, microfluidic device, or the like. The site is within sensory communication of the detector. The phrase “within sensory communication” generally refers to the relative location of the detector that is positioned relative to the site so as to be able to receive a particular relevant signal from that container. In the case of optical detectors, e.g., fluorescence, FRET, or fluorescence polarization detectors, sensory communication typically means that the detector is disposed sufficiently proximal to the container that optical, e.g., fluorescent signals, are transmitted to the detector for adequate detection of those signals. Typically this employs a lens, optical train or other detection element, e.g., a CCD, that is focused upon a relevant portion of the container to efficiently gather and record these optical signals.

Example detectors include patch-clamp stations, photo multiplier tubes, spectrophotometers, a CCD array, a scanning detector, a microscope, a galvo-scann or the like. Cells, dyes or other components which emit a detectable signal can be flowed past or moved into contact with the detector, or, alternatively, the detector can move relative to an array of samples (or, the detector can simultaneously monitor a number of spatial positions corresponding to samples, e.g., as in a CCD array).

The system typically includes a signal detector located proximal to the site of mixing/reaction. The signal detector detects the detectable signal, e.g., for a selected length of time (t). For example, the detector can include a spectrophotometer, or an optical detection element. Commonly, the signal detector is operably coupled to a computer, which deconvolves the detectable signal to provide an indication of the transmembrane potential, e.g., an indication of a change in the potential over time.

The detector can detect transmembrane potential (the work needed to move a unit of charge across a membrane such as a cell membrane) and/or calcium concentration, e.g., through detecting flow of a calcium binding dye, a cationic membrane permeable dye, an anionic Nernstian dye, an anionic membrane permeable dye, or other voltage sensing composition across the membrane over time, e.g., in response to application of a test compound. Changes in the rate of depolarization and hyperpolarization and/or calcium influx are monitored in response to a test (e.g., putative modulator) compound, e.g., as compared to a control that does not include the test compound. Permeable dyes are particularly useful for monitoring ion flow, e.g., dyes that can equilibrate across the membrane relatively quickly, typically in about 1 hour, or less. Permeability can be dependent upon the relevant conditions, e.g., temperature, ionic conditions, voltage potentials, or the like.

Computer

Either or both of the controller system and/or the detection system are optionally coupled to an appropriately programmed processor or computer which functions to instruct the operation of these instruments in accordance with preprogrammed or user input instructions, receive data and information from these instruments, and interpret, manipulate and report this information to the user. As such, the computer is typically appropriately coupled to one or both of these instruments (e.g., including an analog to digital or digital to analog converter as needed).

The computer typically includes appropriate software for receiving user instructions, either in the form of user input into a set parameter fields, e.g., in a GUI, or in the form of preprogrammed instructions, e.g., preprogrammed for a variety of different specific operations. The software then converts these instructions to appropriate language for instructing the operation of the fluid direction and transport controller to carry out the desired operation. The computer then receives the data from the one or more sensors/detectors included within the system, and interprets the data, either provides it in a user understood format, or uses that data to initiate further controller instructions, in accordance with the programming, e.g., such as in monitoring and control of flow rates, temperatures, applied voltages, and the like.

In the present invention, the computer typically includes software for the monitoring of samples. Additionally, the software is optionally used to control flow of materials.

Biosensors

Biosensors of the invention are devices or systems that comprise the polypeptides of the invention (e.g., a Orai or Stim polypeptide or complex) coupled to a readout that measures or displays one or more activity of the polypeptide. Thus, any of the above described assay components can be configured as a biosensor by operably coupling the appropriate assay components to a readout. The readout can be optical (e.g., to detect cell markers, ion-sensitive dyes, cell potential, or cell survival) electrical (e.g., coupled to a FET, a BIAcore, or any of a variety of others), spectrographic, or the like, and can optionally include a user-viewable display (e.g., a CRT or optical viewing station). The biosensor can be coupled to robotics or other automation, e.g., microfluidic systems, that direct contact of the test compounds to the proteins of the invention, e.g., for automated high-throughput analysis of test compound activity. A large variety of automated systems that can be adapted to use with the biosensors of the invention are commercially available. For example, automated systems have been made to assess a variety of biological phenomena, including, e.g., expression levels of genes in response to selected stimuli (Service (1998) “Microchips Arrays Put DNA on the Spot” Science 282:396-399). Laboratory systems can also perform, e.g., repetitive fluid handling operations (e.g., pipetting) for transferring material to or from reagent storage systems that comprise arrays, such as microtiter trays or other chip trays, which are used as basic container elements for a variety of automated laboratory methods. Similarly, the systems manipulate, e.g., microtiter trays and control a variety of environmental conditions such as temperature, exposure to light or air, and the like. Many such automated systems are commercially available. Examples of automated systems are available from Caliper Technologies (including the former Zymark Corporation, Hopkinton, Mass.), which utilize various Zymate systems which typically include, e.g., robotics and fluid handling modules. Similarly, the common ORCA® robot, which is used in a variety of laboratory systems, e.g., for microtiter tray manipulation, is also commercially available, e.g., from Beckman Coulter, Inc. (Fullerton, Calif.). A number of automated approaches to high-throughput activity screening are provided by the Genomics Institute of the Novartis Foundation (La Jolla, Calif.); See GNF.org on the world-wide web. Microfluidic screening applications are also commercially available from Caliper Technologies Corp. For example, (e.g., LabMicrofluidic Device® high throughput screening system (HTS) by Caliper Technologies, Mountain View, Calif. or the HP/Agilent technologies Bioanalyzer using LabChip™ technology by Caliper Technologies Corp. can be adapted for use in the present invention.

In an alternate embodiment, conformational changes are detected by coupling the polypeptides or complexes of the invention to an electrical readout, e.g., to a chemically coupled field effect transistor (a CHEM-FET) or other appropriate system for detecting changes in conductance or other electrical properties brought about by a conformational shift by the protein of the invention.

Further Details Regarding Cells Comprising Orai/Stim

As already noted, for several embodiments, biological samples to be tested for Orai/Stim expression or concentration are cells or are derived from cell preparations. The cells can be those associated with Orai/Stim expression in vivo, such as activated T cells. Alternately, the cells can be derived from such cells, e.g., through culture.

However, one feature of the invention is the production of recombinant cells, e.g., expressing a heterologous orai gene, or both a heterologous orai gene and a heterologous stim gene. In these embodiments, the biological sample to be tested is derived from the recombinant cell, which is selected largely for ease of culture and manipulation. The cells can be, e.g., human, rodent, insect, Xenopus, etc. and will typically be a cell in culture (or an oocyte in the case of Xenopus).

Orai and stim nucleic acids are typically introduced into cells in cloning and/or expression vectors to facilitate introduction of the nucleic acid and expression of orai and/or stim to produce Orai and/or Stim. Vectors include, e.g., plasmids, cosmids, viruses, YACs, bacteria, poly-lysine, etc. A “vector nucleic acid” is a nucleic acid molecule into which a heterologous nucleic acid is optionally inserted which can then be introduced into an appropriate host cell. Vectors preferably have one or more origins of replication, and one or more sites into which the recombinant DNA can be inserted. Vectors often have convenient means by which cells with vectors can be selected from those without, e.g., they encode drug resistance genes. Common vectors include plasmids, viral genomes, and (primarily in yeast and bacteria) artificial chromosomes. “Expression vectors” are vectors that comprise elements that provide for or facilitate transcription of nucleic acids which are cloned into the vectors. Such elements can include, e.g., promoters and/or enhancers operably coupled to a nucleic acid of interest.

In general, appropriate expression vectors are known in the art. For example, pET-14b, pcDNA1Amp, and pVL1392 are available from Novagen and Invitrogen and are suitable vectors for expression in E. coli, COS cells and baculovirus infected insect cells, respectively. pcDNA-3, pEAK, and vectors that permit the generation of orai/stim RNA for in vitro and in vivo expression experiments (e.g., in vitro translations and Xenopus oocyte injections) are also useful. These vectors are illustrative of those that are known in the art. Suitable host cells can be any cell capable of growth in a suitable media and allowing purification of the expressed protein. Examples of suitable host cells include bacterial cells, such as E. coli, Streptococci, Staphylococci, Streptomyces and Bacillus subtilis cells; fungal cells such as yeast cells, e.g., Pichia, and Aspergillus cells; insect cells such as Drosophila S2 and Spodoptera Sf9 cells, mammalian cells such as CHO, COS, and HeLa; and even plant cells.

Cells are transformed with orai and/or stim genes according to standard cloning and transformation methods. Such genes can also be isolated from resulting recombinant cells using standard methods. General texts which describe molecular biological techniques for making nucleic acids, including the use of vectors, promoters and many other relevant topics, include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3nd Ed.), Vol. 1-3, Cold Spring Harbor Laboratory, Cold Spring Harbor, N.Y., 2000 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 2002) (“Ausubel”)).

In addition, a plethora of kits are commercially available for the preparation, purification and cloning of plasmids or other relevant nucleic acids from cells, (see, e.g., EasyPrep™, FlexiPrep™, both from Pharmacia Biotech; StrataClean™, from Stratagene; and, QIAprep™ from Qiagen). Any isolated and/or purified nucleic acid can be further manipulated to produce other nucleic acids, used to transfect cells, incorporated into related vectors to infect organisms, or the like.

As noted, typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or both. See, Giliman & Smith, Gene 8:81 (1979); Roberts, et al., Nature, 328:731 (1987); Schneider, B., et al., Protein Expr. Purif. 6435:10 (1995); Ausubel, Sambrook, Berger (above). A catalogue of Bacteria and Bacteriophages useful for cloning is provided, e.g., by the ATCC, e.g., The ATCC Catalogue of Bacteria and Bacteriophage published yearly by the ATCC. Additional basic procedures for sequencing, cloning and other aspects of molecular biology and underlying theoretical considerations are also found in Watson et al. (1992) Recombinant DNA Second Edition, Scientific American Books, NY.

In addition, essentially any nucleic acid (and virtually any labeled nucleic acid, whether standard or non-standard) can be custom or standard ordered from any of a variety of commercial sources, such as The Midland Certified Reagent Company (mcrc@oligos.com), The Great American Gene Company (www.genco.com), ExpressGen Inc. (www.expressgen.com), Operon Technologies Inc. (Alameda, Calif.) and many others.

Other useful references, e.g. for cell isolation and culture (e.g., for subsequent nucleic acid isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York); and Atlas and Parks (eds) The Handbook of Microbiological Media (1993) CRC Press, Boca Raton, Fla.

Additional Details Regarding Protein Purification and Handling

Purification of Orai and/or Stim, can be accomplished using known techniques. Generally, when purification is desired, e.g., for cell free assays, transformed cells expressing Orai and/or Stim are lysed, crude purification occurs to remove debris and contaminating proteins, followed by chromatography to further purify the protein to the desired level of purity. Cells can be lysed by known techniques such as homogenization, sonication, detergent lysis and freeze-thaw techniques. Crude purification can occur using ammonium sulfate precipitation, centrifugation or other known techniques. Suitable chromatography includes anion exchange, cation exchange, high performance liquid chromatography (HPLC), gel filtration, affinity chromatography, hydrophobic interaction chromatography, etc. Well known techniques for refolding proteins can be used to obtain the active conformation of the protein when the protein is denatured during intracellular synthesis, isolation or purification.

In general, Orai or Stim polypeptides, can be purified, either partially (e.g., achieving a 5×, 10×, 100×, 500×, or 1000× or greater purification), or even substantially to homogeneity (e.g., where the protein is the main component of a solution, typically excluding the solvent (e.g., water or DMSO) and buffer components (e.g., salts and stabilizers) that the polypeptide is suspended in, e.g., if the polypeptide is in a liquid phase), according to standard procedures known to and used by those of skill in the art. Accordingly, polypeptides of the invention can be recovered and purified by any of a number of methods well known in the art, including, e.g., immunoprecipitations, ammonium sulfate or ethanol precipitation, acid or base extraction, column chromatography, affinity column chromatography, anion or cation exchange chromatography, phosphocellulose chromatography, hydrophobic interaction chromatography, hydroxylapatite chromatography, lectin chromatography, gel electrophoresis and the like. Protein refolding steps can be used, as desired, in making correctly folded mature proteins. High performance liquid chromatography (HPLC), affinity chromatography or other suitable methods can be employed in final purification steps where high purity is desired. In one example embodiment, antibodies made against Orai and/or Stim are used as purification reagents, e.g., for affinity-based purification (e.g., by immunoprecipitations). Once purified, partially or to homogeneity, as desired, the polypeptides are optionally used e.g., as assay components, therapeutic reagents or as immunogens for antibody production.

In addition to other references noted herein, a variety of purification/protein purification methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2nd Edition Wiley-Liss, NY; Walker (1996) The Protein Protocols Handbook Humana Press, NJ; Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3rd Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ; and the references cited therein.

Those of skill in the art will recognize that, after synthesis, expression and/or purification, proteins can possess a conformation different from the desired conformations of the relevant polypeptides. For example, polypeptides produced by prokaryotic systems often are optimized by exposure to chaotropic agents to achieve proper folding. During purification from, e.g., lysates derived from E. coli, the expressed protein is optionally denatured and then renatured. This is accomplished, e.g., by solubilizing the proteins in a chaotropic agent such as guanidine HCl. In general, it is occasionally desirable to denature and reduce expressed polypeptides and then to cause the polypeptides to re-fold into the preferred conformation. For example, guanidine, urea, DTT, DTE, and/or a chaperonin can be added to a translation product of interest. Methods of reducing, denaturing and renaturing proteins are well known to those of skill in the art (see, the references above, and Debinski, et al. (1993) J. Biol. Chem., 268: 14065-14070; Kreitman and Pastan (1993) Bioconjug. Chem., 4: 581-585; and Buchner, et al., (1992) Anal. Biochem., 205: 263-270). Debinski, et al., for example, describe the denaturation and reduction of inclusion body proteins in guanidine-DTE. The proteins can be refolded in a redox buffer containing, e.g., oxidized glutathione and L-arginine. Refolding reagents can be flowed or otherwise moved into contact with the one or more polypeptide or other expression product, or vice-versa.

Orai and stim nucleic acids optionally comprise a coding sequence fused in-frame to a marker sequence which, e.g., facilitates purification of the encoded polypeptide. Such purification facilitating domains include, but are not limited to, metal chelating peptides such as histidine-tryptophan modules that allow purification on immobilized metals, a sequence which binds glutathione (e.g., GST), a hemagglutinin (HA) tag (corresponding to an epitope derived from the influenza hemagglutinin protein; Wilson, I., et al. (1984) Cell 37:767), maltose binding protein sequences, the FLAG epitope utilized in the FLAGS extension/affinity purification system (Immunex Corp, Seattle, Wash.), and the like. See also, the Examples sections below. The inclusion of a protease-cleavable polypeptide linker sequence between the purification domain and the sequence of the invention is useful to facilitate purification.

Making Knock-Out Animals and Transgenics

In one aspect, the invention includes knock out and/or transgenic animals. For example, non-human laboratory animals that comprise a knock out in an endogenous Orai and/or Stim gene can be made and can additionally include a heterologous Orai or Stim gene (e.g., from a human source) corresponding to the knock out.

A transgenic animal is typically an animal that has had DNA introduced into one or more of its cells artificially. This is most commonly done in one of two ways. First, DNA can be integrated randomly by injecting it into the pronucleus of a fertilized ovum. In this case, the DNA can integrate anywhere in the genome. In this approach, there is no need for homology between the injected DNA and the host genome. Second, targeted insertion can be accomplished by introducing heterologous DNA into embryonic stem (ES) cells and selecting for cells in which the heterologous DNA has undergone homologous recombination with homologous sequences of the cellular genome. Typically, there are several kilobases of homology between the heterologous and genomic DNA, and positive selectable markers (e.g., antibiotic resistance genes) are included in the heterologous DNA to provide for selection of transformants. In addition, negative selectable markers (e.g., “toxic” genes such as barnase) can be used to select against cells that have incorporated DNA by non-homologous recombination (i.e., random insertion).

One common use of targeted insertion of DNA is to make knock-out mice. Mice provide a very useful laboratory animal, due to the ease with which the animals can be bread, made recombinant, etc. Typically, when making knock outs in mice (or other laboratory animals), homologous recombination is used to insert a selectable gene driven by a constitutive promoter into an essential exon of the gene that one wishes to disrupt (e.g., the first coding exon). To accomplish this, the selectable marker is flanked by large stretches of DNA that match the genomic sequences surrounding the desired insertion point. Once this construct is electroporated into ES cells, the cells' own machinery performs the homologous recombination. To make it possible to select against ES cells that incorporate DNA by non-homologous recombination, it is common for targeting constructs to include a negatively selectable gene outside the region intended to undergo recombination (typically the gene is cloned adjacent to the shorter of the two regions of genomic homology). Because DNA lying outside the regions of genomic homology is lost during homologous recombination, cells undergoing homologous recombination cannot be selected against, whereas cells undergoing random integration of DNA often can. A commonly used gene for negative selection is the herpes virus thymidine kinase gene, which confers sensitivity to the drug gancyclovir.

Following positive selection and negative selection if desired, ES cell clones are screened for incorporation of the construct into the correct genomic locus. Typically, one designs a targeting construct so that a band normally seen on a Southern blot or following PCR amplification becomes replaced by a band of a predicted size when homologous recombination occurs. Since ES cells are diploid, only one allele is usually altered by the recombination event so, when appropriate targeting has occurred, one usually sees bands representing both wild type and targeted alleles.

The embryonic stem (ES) cells that are used for targeted insertion are derived from the inner cell masses of blastocysts (early mouse embryos). These cells are pluripotent, meaning they can develop into any type of tissue.

Once positive ES clones have been grown up and frozen, the production of transgenic animals can begin. Donor females are mated, blastocysts are harvested, and several ES cells are injected into each blastocyst. Blastocysts are then implanted into a uterine horn of each recipient. By choosing an appropriate donor strain, the detection of chimeric offspring (i.e., those in which some fraction of tissue is derived from the transgenic ES cells) can be as simple as observing hair and/or eye color. If the transgenic ES cells do not contribute to the germline (sperm or eggs), the transgene cannot be passed on to offspring.

Transgenic animals are a useful tool for studying gene function and testing modulators. Human (or other selected) orai or stim genes can be introduced in place of endogenous orai or stim genes of a laboratory animal, making it possible to study function of the human (or other) polypeptide or complex in the easily manipulated and studied laboratory animal. It will be appreciated that there is not precise correspondence between receptor structure or function of different animals, making the ability to study the human or other receptor of interest particularly useful when developing clinical candidate modulators. Although similar genetic manipulations can be performed in tissue culture, the interaction of Orai/Stim in the context of an intact organism provides a more complete and physiologically relevant picture of function than could be achieved in simple cell-based screening assays. Accordingly, knock-out transgenic animals are particularly useful when analyzing modulators identified in high throughput in vitro (e.g., cell-based) systems.

Cell Rescue—Treatment

In one aspect, the invention includes rescue of a cell that is defective in function of one or more endogenous orai or stim genes or polypeptides. This can be accomplished simply by introducing a new copy of the gene (or a heterologous nucleic acid that expresses the relevant protein) into a cell. Other approaches, such as homologous recombination to repair the defective gene (e.g., via chimeraplasty) can also be performed. In any event, rescue of function can be measured, e.g., in any of the assays noted herein. Indeed, this can be used as a general method of screening cells in vitro for activity. Accordingly, in vitro rescue of function is useful in this context for the myriad in vitro screening methods noted above, e.g., for the identification of modulators in cells. The cells that are rescued can include cells in culture, (including primary or secondary cell culture from patients, as well as cultures of well-established cells). Where the cells are isolated from a patient, this has additional diagnostic utility in establishing which sequence is defective in a patient that presents with a CRAC channel defect.

In another aspect, cell rescue occurs in a patient, e.g., a human or veterinary patient, e.g., to remedy a CRAC channel defect. Thus, one aspect of the invention is gene therapy to remedy defects, in human or veterinary applications. In these applications, the nucleic acids of the invention are optionally cloned into appropriate gene therapy vectors (and/or are simply delivered as naked or liposome-conjugated nucleic acids), which are then delivered, optionally in combination with appropriate carriers or delivery agents. Proteins can also be delivered directly, but delivery of the nucleic acid is typically preferred in applications where stable expression is desired.

Vectors for administration typically comprise orai and/or stim genes under the control of a promoter that is expressed in a cell of interest (e.g., a T-cell). These can include native orai and/or stim promoters and/or upstream regulatory elements, or other cell specific promoters, such T-cell specific promoters.

Compositions for administration, e.g., comprise a therapeutically effective amount of the gene therapy vector or other relevant nucleic acid, and a pharmaceutically acceptable carrier or excipient. Such a carrier or excipient includes, but is not limited to, saline, buffered saline, dextrose, water, glycerol, ethanol, and/or combinations thereof. The formulation is made to suit the mode of administration. In general, methods of administering gene therapy vectors for topical use are well known in the art and can be applied to administration of the nucleic acids of the invention.

Therapeutic compositions comprising one or more nucleic acid of the invention are optionally tested in one or more appropriate in vitro and/or in vivo animal model of disease, to confirm efficacy, tissue metabolism, and to estimate dosages, according to methods well known in the art. In particular, dosages can initially be determined by activity, stability or other suitable measures of the formulation.

Administration is by any of the routes normally used for introducing a molecule into ultimate contact with cells of interest. Practitioners can select an administration route of interest based on the cell target. For example, intravenous administration is one way of introducing genes into contact with T-cells. T-cells and other blood cells can also be easily manipulated ex vivo and later returned to the patient intravenously.

The nucleic acids of the invention are administered in any suitable manner, optionally with one or more pharmaceutically acceptable carriers. Suitable methods of administering such nucleic acids in the context of the present invention to a patient are available, and, although more than one route can be used to administer a particular composition, a particular route can often provide a more immediate and more effective action or reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention. Compositions can be administered by a number of routes including, but not limited to: oral, intravenous, intraperitoneal, intramuscular, transdermal, subcutaneous, topical, sublingual, spinal, or rectal administration. Compositions can be administered via liposomes (e.g., topically), or via topical delivery of naked DNA or viral vectors. Such administration routes and appropriate formulations are generally known to those of skill in the art.

The compositions, alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and the like. Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations of packaged nucleic acid can be presented in unit-dose or multi-dose sealed containers, such as ampules and vials.

The dose administered to a patient, in the context of the present invention, is sufficient to effect a beneficial therapeutic response in the patient over time. The dose is determined by the efficacy of the particular vector, or other formulation, and the activity, stability or serum half-life of the polypeptide which is expressed, and the condition of the patient, as well as the body weight or surface area of the patient to be treated. The size of the dose is also determined by the existence, nature, and extent of any adverse side-effects that accompany the administration of a particular vector, formulation, or the like in a particular patient. In determining the effective amount of the vector or formulation to be administered in the treatment of disease, the physician evaluates local expression in the tissue or cell of interest, or circulating plasma levels, formulation toxicities, progression of the relevant disease, and/or where relevant, the production of antibodies to proteins encoded by the polynucleotides. The dose administered, e.g., to a 70 kilogram patient are typically in the range equivalent to dosages of currently-used therapeutic proteins, adjusted for the altered activity or serum half-life of the relevant composition. The vectors of this invention can supplement treatment conditions by any known conventional therapy (e.g., diet restriction, etc.).

For administration, formulations of the present invention are administered at a rate determined by the LD-50 of the relevant formulation, and/or observation of any side-effects of the vectors of the invention at various concentrations, e.g., as applied to the mass or topical delivery area and overall health of the patient. Administration can be accomplished via single or divided doses.

If a patient undergoing treatment develops fevers, chills, or muscle aches, he/she receives the appropriate dose of aspirin, ibuprofen, acetaminophen or other pain/fever controlling drug. Patients who experience reactions to the compositions, such as fever, muscle aches, and chills are premedicated 30 minutes prior to the future infusions with either aspirin, acetaminophen, or, e.g., diphenhydramine. Meperidine is used for more severe chills and muscle aches that do not quickly respond to antipyretics and antihistamines. Treatment is slowed or discontinued depending upon the severity of the reaction.

Additional Details Regarding Treatment

In one aspect of the invention, the subject methods can be used as part of a treatment regimen for an immunological disorder in a subject to restore CRAC channel activity. As used herein “immunological disorder” refers to any disorder of the immune system. A non-limiting example of an immunological disorder that can be treated by the methods of the invention is Severe Combined Immunodeficiency (SCID), which is a primary immune deficiency. The defining characteristic is usually a severe defect in both the T- & B-lymphocyte systems. This usually results in the onset of one or more serious infections within the first few months of life. These infections are usually serious, and may even be life threatening, they may include pneumonia, meningitis or bloodstream infections. As such, the methods of the invention include administering to the subject a therapeutically effective amount of a protein identified as a SOC regulator.

In another aspect of the invention, the subject methods can be used as part of a treatment regimen for a CRAC-associated disorder. The term “disorder” or “disease” as used herein refers to any condition associated with calcium influx through a calcium channel. CRAC-associated disorders include, but are not limited to, kidney disorders, cardiovascular disorders, immunological disorders, and angiogenesis-related disorders. Other related conditions include atherosclerosis and systemic sclerosis, including atherosclerotic plaques, inflammatory bowel disease, Crohn's disease, angiogenesis, and other proliferative processes which play central roles in atherosclerosis, arthritis, cancer, and other disease states; neovascularization involved in glaucoma, inflammation due to disease or injury, including joint inflammation, tumor growth metastasis, interstitial disease; dermatological diseases; asthma; hepatitis; lupus; acquired immune deficiency syndrome (AIDS); multiple sclerosis (MS); Alzheimers, psoriasis; meningitis; neurodegeneration; cachexia; euthyroid sick syndrome; glomerulonephritis; arthritis, including chronic rheumatoid arthritis, arteriosclerosis; diabetes, including diabetic nephropathy and retinopathy, hypertension, and other kidney disorders; ischemia/reperfusion injury; and fibrosis resulting from chemotherapy, radiation treatment, dialysis, and allograft and transplant rejection.

The terms “cell proliferative disorder” or “cellular proliferative disorder” refer to any disorder in which the proliferative capabilities of the affected cells is different from the normal proliferative capabilities of unaffected cells. An example of a cell proliferative disorder is neoplasia. Malignant cells (e.g., cancer cells) develop as a result of a multistep process. The term “cancer” as used herein, includes any malignant tumor including, but not limited to, carcinoma and sarcoma. Cancer arises from the uncontrolled and/or abnormal division of cells that then invade and destroy the surrounding tissues. As used herein, “proliferating” and “proliferation” refer to cells undergoing mitosis. As used herein, “metastasis” refers to the distant spread of a malignant tumor from its sight of origin. Cancer cells may metastasize through the bloodstream, through the lymphatic system, across body cavities, or any combination thereof. The term “cancerous cell” as provided herein, includes a cell afflicted by any one of the cancerous conditions provided herein. The term “carcinoma” refers to a malignant new growth made up of epithelial cells tending to infiltrate surrounding tissues, and to give rise to metastases.

A cell proliferative disorder as described herein may be a neoplasm. Such neoplasms are either benign or malignant. The term “neoplasm” refers to a new, abnormal growth of cells or a growth of abnormal cells that reproduce faster than normal. A neoplasm creates an unstructured mass (a tumor) which can be either benign or malignant. For example, the neoplasm may be a head, neck, lung, esophageal, stomach, small bowel, colon, bladder, kidney, or cervical neoplasm. The term “benign” refers to a tumor that is noncancerous, e.g. its cells do not proliferate or invade surrounding tissues. The term “malignant” refers to a tumor that is metastatic or no longer under normal cellular growth control.

A cell proliferative disorder may further be an age-associated disorder. Examples of age-associated disorders which are cell proliferative disorders include colon cancer, lung cancer, breast cancer, prostate cancer, and melanoma, amongst others.

The term “patient” or “subject” as used herein refers to any individual to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

The term “therapeutically effective amount” or “effective amount” means the amount of a compound or pharmaceutical composition that will elicit the biological or medical response of a tissue, system, animal or human that is being sought by the researcher, veterinarian, medical doctor or other clinician.

The term “pharmaceutically acceptable”, when used in reference to a carrier, is meant that the carrier, diluent or excipient must be compatible with the other ingredients of the formulation and not deleterious to the recipient thereof.

The terms “administration” or “administering” is defined to include an act of providing a compound or pharmaceutical composition of the invention to a subject in need of treatment. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. The phrases “systemic administration,” “administered systemically,” “peripheral administration” and “administered peripherally” as used herein mean the administration of a compound, drug or other material other than directly into the central nervous system, such that it enters the subject's system and, thus, is subject to metabolism and other like processes, for example, subcutaneous administration.

The efficacy of a therapeutic method of the invention over time can be identified by an absence of symptoms or clinical signs of an immunological disorder in a subject predisposed to the disorder, but not yet exhibiting the signs or symptoms of the disorder at the time of onset of therapy. In subjects diagnosed as having the immunological disorder, or other condition in which it is desirable to modulate the immune response, the efficacy of a method of the invention can be evaluated by measuring a lessening in the severity of the signs or symptoms in the subject or by the occurrence of a surrogate end-point for the disorder.

As used herein “corresponding normal cells” means cells that are from the same organ and of the same type as the cells being examined. In one aspect, the corresponding normal cells comprise a sample of cells obtained from a healthy individual. Such corresponding normal cells can, but need not be, from an individual that is age-matched and/or of the same sex as the individual providing the cells being examined.

As used herein, the terms “sample” and “biological sample” refer to any sample suitable for the methods provided by the present invention. In one embodiment, the biological sample of the present invention is a tissue sample, e.g., a biopsy specimen such as samples from needle biopsy. In other embodiments, the biological sample of the present invention is a sample of bodily fluid, e.g., serum, plasma, urine, and ejaculate.

As used herein, the terms “reduce” and “inhibit” are used together because it is recognized that, in some cases, a decrease, for example, in Ca²⁺ influx can be reduced below the level of detection of a particular assay. As such, it may not always be clear whether the activity is “reduced” below a level of detection of an assay, or is completely “inhibited”.

Detecting Polymorphisms

In one aspect, the invention includes detecting a polymorphism in an orai or stim gene (or a nucleic acid in linkage disequilibrium with such a polymorphism) to detect a CRAC channel abnormality caused by a polymorphism in these genes. For example, SCID is associated with a mutation in the human orai gene.

A “polymorphism” is a locus that is variable; that is, within a population, the nucleotide sequence at a polymorphism has more than one version or allele. The term “allele” refers to one of two or more different nucleotide sequences that occur or are encoded at a specific locus, or two or more different polypeptide sequences encoded by such a locus. For example, a first allele can occur on one chromosome, while a second allele occurs on a second homologous chromosome, e.g., as occurs for different chromosomes of a heterozygous individual, or between different homozygous or heterozygous individuals in a population. One example of a polymorphism is a “single nucleotide polymorphism” (SNP), which is a polymorphism at a single nucleotide position in a genome (the nucleotide at the specified position varies between individuals or populations). An allele “positively” correlates with a trait when it is linked to it and when presence of the allele is an indictor that the trait or trait form will occur in an individual comprising the allele. An allele negatively correlates with a trait when it is linked to it and when presence of the allele is an indicator that a trait or trait form will not occur in an individual comprising the allele.

In the present case, genes for CRAC channel genes are identified (orai, stim). Polymorphisms within or linked to (in linkage disequilibrium with) these genes likely correlate to altered immune function, predisposition to cancer, and a variety of other clinically relevant conditions. Disease conditions or predispositions arising from CRAC channel polymorphisms can be detected by detecting polymorphisms in the relevant gene.

In general, markers corresponding to polymorphisms between members of a population can be detected by numerous methods well-established in the art (e.g., PCR-based sequence specific amplification, restriction fragment length polymorphisms (RFLPs), isozyme markers, northern analysis, allele specific hybridization (ASH), array based hybridization, amplified variable sequences of the genome, self-sustained sequence replication, simple sequence repeat (SSR), single nucleotide polymorphism (SNP), random amplified polymorphic DNA (“RAPD”) or amplified fragment length polymorphisms (AFLP). In one additional embodiment, the presence or absence of a molecular marker is determined simply through nucleotide sequencing of the polymorphic marker region. Any of these methods are readily adapted to high throughput analysis.

Additional Details Regarding Sequence Variations

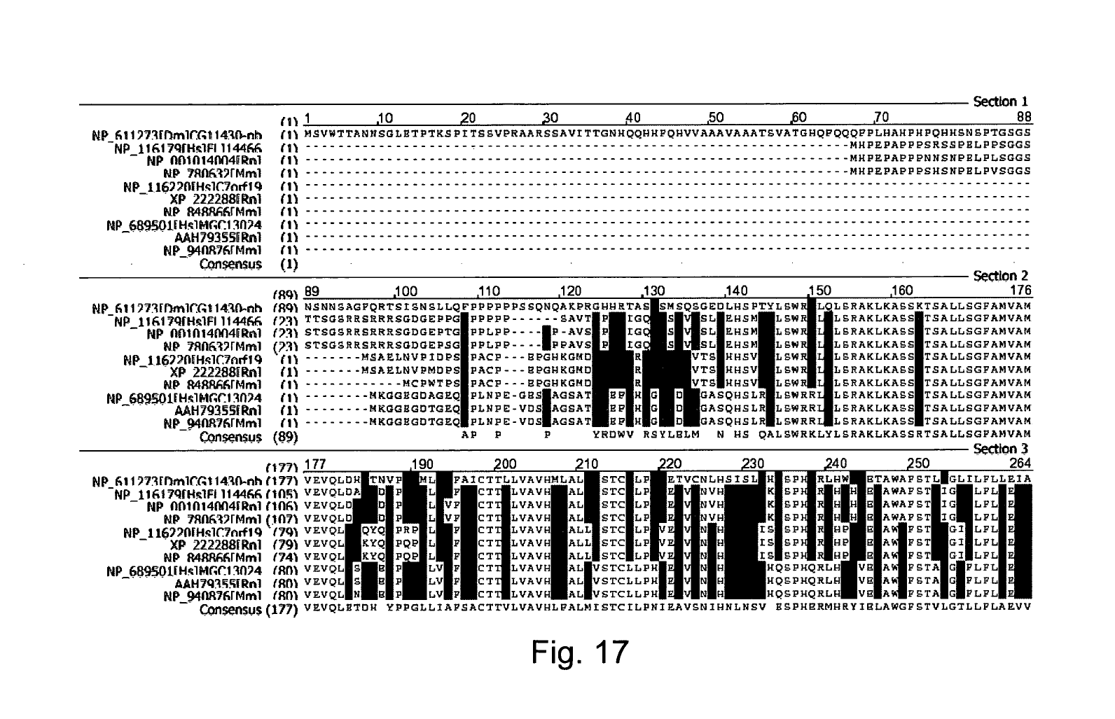

A number of particular Orai and Stim polypeptides and coding nucleic acids are described herein by sequence (See, e.g., the Examples section below; Table 1A, FIG. 8A, FIGS. 17-19). These polypeptides and coding nucleic acids can be modified, e.g., by mutation as described herein, or simply by artificial synthesis of a desired variant. Several types of example variants are described below.

Splice Variants

Splice variants of Orai and Stim may exist. These can be expressed alone or in combination and can be detected or monitored by analysis of mRNA using exon-specific primers and the polymerase chain reaction.

Silent Variations

Due to the degeneracy of the genetic code, any of a variety of nucleic acid sequences encoding polypeptides of the invention are optionally produced, some which can bear sequence identity to the orai or stim nucleic acids in the Examples herein. The following provides a typical codon table specifying the genetic code, found in many biology and biochemistry texts.

TABLE A
Codon Table

| | |
|---|---|
| Amino acids | Codon |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Praline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

The codon table shows that many amino acids are encoded by more than one codon. For example, the codons AGA, AGG, CGA, CGC, CGG, and CGU all encode the amino acid arginine. Thus, at every position in the nucleic acids of the invention where an arginine is specified by a codon, the codon can be altered to any of the corresponding codons described above without altering the encoded polypeptide. It is understood that U in an RNA sequence corresponds to T in a DNA sequence.

Such “silent variations” are one species of “conservatively modified variations”, discussed below. One of skill will recognize that each codon in a nucleic acid (except ATG, which is ordinarily the only codon for methionine) can be modified by standard techniques to encode a functionally identical polypeptide. Accordingly, each silent variation of a nucleic acid which encodes a polypeptide is implicit in any described sequence. The invention, therefore, explicitly provides each and every possible variation of a nucleic acid sequence encoding a polypeptide of the invention that could be made by selecting combinations based on possible codon choices. These combinations are made in accordance with the standard triplet genetic code (e.g., as set forth in Table A, or as is commonly available in the art) as applied to the nucleic acid sequence encoding a Orai or Stim polypeptide of the invention. All such variations of every nucleic acid herein are specifically provided and described by consideration of the sequence in combination with the genetic code. One of skill is fully able to make these silent substitutions using the methods herein.

Conservative Variations

“Conservatively modified variations” or, simply, “conservative variations” of a particular nucleic acid sequence or polypeptide are those which encode identical or essentially identical amino acid sequences. One of skill will recognize that individual substitutions, deletions or additions which alter, add or delete a single amino acid or a small percentage of amino acids (typically less than 5%, more typically less than 4%, 2% or 1%) in an encoded sequence are “conservatively modified variations” where the alterations result in the deletion of an amino acid, addition of an amino acid, or substitution of an amino acid with a chemically similar amino acid.

Conservative substitution tables providing functionally similar amino acids are well known in the art. Table B sets forth six groups which contain amino acids that are “conservative substitutions” for one another.

TABLE B
Conservative Substitution Groups

| | | | | |
|---|---|---|---|---|
| 1 | Alanine (A) | Serine (S) | Threonine (T) | |
| 2 | Aspartic acid (D) | Glutamic acid (E) | | |
| 3 | Asparagine (N) | Glutamine (Q) | | |
| 4 | Arginine (R) | Lysine (K) | | |
| 5 | Isoleucine (I) | Leucine (L) | Methionine (M) | Valine (V) |
| 6 | Phenylalanine (F) | Tyrosine (Y) | Tryptophan (W) | |

Thus, “conservatively substituted variations” of a listed polypeptide sequence of the present invention include substitutions of a small percentage, typically less than 5%, more typically less than 2% or 1%, of the amino acids of the polypeptide sequence, with a conservatively selected amino acid of the same conservative substitution group.

Finally, the addition or deletion of sequences which do not alter the encoded activity of a nucleic acid molecule, such as the addition or deletion of a non-functional sequence, is a conservative variation of the basic nucleic acid or polypeptide.

One of skill will appreciate that many conservative variations of the nucleic acid constructs which are disclosed yield a functionally identical construct. For example, as discussed above, owing to the degeneracy of the genetic code, “silent substitutions” (i.e., substitutions in a nucleic acid sequence which do not result in an alteration in an encoded polypeptide) are an implied feature of every nucleic acid sequence which encodes an amino acid. Similarly, “conservative amino acid substitutions,” in one or a few amino acids in an amino acid sequence are substituted with different amino acids with highly similar properties, are also readily identified as being highly similar to a disclosed construct. Such conservative variations of each disclosed sequence are a feature of the present invention.

Further Details Regarding Orai and Stim Variants

Any of a variety of Orai and Stim polypeptides and coding nucleic acids can be used in the present invention. Examples of such polypeptides and coding genes are available, including those in Table 1A. In Table 1A, the first column lists accession numbers for Orai family members in different species. Orai is one name for the family of genes related to Drosophila olf186-F; another name for Orai1 is CRACM1. CaSOC is a proposed nomenclature that stands for Ca²⁺-selective, Store (or Stim)-Operated (or Orai) Current, to represent its function as a store-operated Ca²⁺ channel; human Orai1 would be hCaSOC1. Accession numbers can be searched in the NCBI data base.

TABLE 1A
Orai Family genes

| Orai family | |
|---|---|
| NP_116179[Hs] | hOrai1/hCaSOC1 |
| NP_116220[Hs] | hOrai2/hCaSOC2 |
| NP_689501[Hs] | hOrai3/hCaSOC3 |
| XP_543386[Cf] | |
| XP_850105[Cf] | |
| XP_849021[Cf] | |
| NP_780632[Mm] | |
| XP_930418[Mm] | |
| NP_940816[Mm] | |
| AAH88225[Rn] | |
| AAH66070[Rn] | |
| AAH79355[Rn] | |
| CAG31281[Gg] | |
| CAG31588[Gg] | |
| XP_706744[Dr] | |
| AAH94965[Dr] | |
| XP_695046[Dr] | |
| AAM68473[Dm] | Orai/dCaSOC |
| NP_497231[Ce] | |
| CAA93402[Sc] | |

Hs: human
Cf: dog
Mm: mouse
Rn: rat
Gg: chicken
Dr: zibra fish
Dm: fly
Ce: worm
Sc: budding yeast
Note:
XP_930418 and AAH66070 are almost identical. the latter appears in both mouse and rat databases.

Examples of Stim and Orai sequences are provided in the Examples section below and in FIGS. 8, 14, and 16-19 and are further available in public databases, e.g., as noted above.

The sequence of any available orai or stim genes and coded polypeptides can be modified by standard methods to provide variants of such available sequences, including conservative or non-conservative variants. Any available mutagenesis procedure can be used to modify a relevant gene. Such mutagenesis procedures optionally include selection of mutant nucleic acids and polypeptides for one or more activity of interest (e.g., increased or decreased responsiveness to CRAC channel stimuli, an alteration in ion conductivity, or the like). Procedures that can be used include, but are not limited to: site-directed point mutagenesis, random point mutagenesis, in vitro or in vivo homologous recombination (DNA shuffling), mutagenesis using uracil containing templates, oligonucleotide-directed mutagenesis, phosphorothioate-modified DNA mutagenesis, mutagenesis using gapped duplex DNA, point mismatch repair, mutagenesis using repair-deficient host strains, restriction-selection and restriction-purification, deletion mutagenesis, mutagenesis by total gene synthesis, double-strand break repair, and many others known to persons of skill. Mutagenesis, e.g., involving chimeric constructs, are also included in the present invention. In one embodiment, mutagenesis can be guided by known information of the naturally occurring molecule or altered or mutated naturally occurring molecule, e.g., sequence, sequence comparisons, physical properties, crystal structure or the like. In another class of embodiments, modification is essentially random (e.g., as in classical DNA shuffling).

Additional information regarding mutation is found in the following publications and references cited within: Arnold, Protein engineering for unusual environments, Current Opinion in Biotechnology 4:450-455 (1993); Bass et al., Mutant Trp repressors with new DNA-binding specificities, Science 242:240-245 (1988); Botstein & Shortle, Strategies and applications of in vitro mutagenesis, Science 229:1193-1201 (1985); Carter et al., Improved oligonucleotide site-directed mutagenesis using M13 vectors, Nucl. Acids Res. 13: 4431-4443 (1985); Carter, Site-directed mutagenesis, Biochem. J. 237:1-7 (1986); Carter, Improved oligonucleotide-directed mutagenesis using M13 vectors, Methods in Enzymol. 154: 382-403 (1987); Dale et al., Oligonucleotide-directed random mutagenesis using the phosphorothioate method, Methods Mol. Biol. 57:369-374 (1996); Eghtedarzadeh & Henikoff, Use of oligonucleotides to generate large deletions, Nucl. Acids Res. 14: 5115 (1986); Fritz et al., Oligonucleotide-directed construction of mutations: a gapped duplex DNA procedure without enzymatic reactions in vitro, Nucl. Acids Res. 16: 6987-6999 (1988); Grundström et al., Oligonucleotide-directed mutagenesis by microscale ‘shot-gun’ gene synthesis, Nucl. Acids Res. 13: 3305-3316 (1985); Kunkel, The efficiency of oligonucleotide directed mutagenesis, in Nucleic Acids & Molecular Biology (Eckstein, F. and Lilley, D. M. J. eds., Springer Verlag, Berlin)) (1987); Kunkel, Rapid and efficient site-specific mutagenesis without phenotypic selection, Proc. Natl. Acad. Sci. USA 82:488-492 (1985); Kunkel et al., Rapid and efficient site-specific mutagenesis without phenotypic selection, Methods in Enzymol. 154, 367-382 (1987); Kramer et al., The gapped duplex DNA approach to oligonucleotide-directed mutation construction, Nucl. Acids Res. 12: 9441-9456 (1984); Kramer & Fritz Oligonucleotide-directed construction of mutations via gapped duplex DNA, Methods in Enzymol. 154:350-367 (1987); Kramer et al., Point Mismatch Repair, Cell 38:879-887 (1984); Kramer et al., Improved enzymatic in vitro reactions in the gapped duplex DNA approach to oligonucleotide-directed construction of mutations, Nucl. Acids Res. 16: 7207 (1988); Ling et al., Approaches to DNA mutagenesis: an overview, Anal Biochem. 254(2): 157-178 (1997); Lorimer and Pastan Nucleic Acids Res. 23, 3067-8 (1995); Mandecki, Oligonucleotide-directed double-strand break repair in plasmids of Escherichia coli: a method for site-specific mutagenesis, Proc. Natl. Acad. Sci. USA, 83:7177-7181 (1986); Nakamaye & Eckstein, Inhibition of restriction endonuclease Nci I cleavage by phosphorothioate groups and its application to oligonucleotide-directed mutagenesis, Nucl. Acids Res. 14: 9679-9698 (1986); Nambiar et al., Total synthesis and cloning of a gene coding for the ribonuclease S protein, Science 223: 1299-1301 (1984); Sakamar and Khorana, Total synthesis and expression of a gene for the a-subunit of bovine rod outer segment guanine nucleotide-binding protein (transducin), Nucl. Acids Res. 14: 6361-6372 (1988); Sayers et al., Y-T Exonucleases in phosphorothioate-based oligonucleotide-directed mutagenesis, Nucl. Acids Res. 16:791-802 (1988); Sayers et al., Strand specific cleavage of phosphorothioate-containing DNA by reaction with restriction endonucleases in the presence of ethidium bromide, (1988) Nucl. Acids Res. 16: 803-814; Sieber, et al., Nature Biotechnology, 19:456-460 (2001); Smith, In vitro mutagenesis, Ann. Rev. Genet. 19:423-462 (1985); Methods in Enzymol. 100: 468-500 (1983); Methods in Enzymol. 154: 329-350 (1987); Stemmer, Nature 370, 389-91 (1994); Taylor et al., The use of phosphorothioate-modified DNA in restriction enzyme reactions to prepare nicked DNA, Nucl. Acids Res. 13: 8749-8764 (1985); Taylor et al., The rapid generation of oligonucleotide-directed mutations at high frequency using phosphorothioate-modified DNA, Nucl. Acids Res. 13: 8765-8787 (1985); Wells et al., Importance of hydrogen-bond formation in stabilizing the transition state of subtilisin, Phil. Trans. R. Soc. Lond. A 317: 415-423 (1986); Wells et al., Cassette mutagenesis: an efficient method for generation of multiple mutations at defined sites, Gene 34:315-323 (1985); Zoller & Smith, Oligonucleotide-directed mutagenesis using M13-derived vectors: an efficient and general procedure for the production of point mutations in any DNA fragment, Nucleic Acids Res. 10:6487-6500 (1982); Zoller & Smith, Oligonucleotide-directed mutagenesis of DNA fragments cloned into M13 vectors, Methods in Enzymol. 100:468-500 (1983); and Zoller & Smith, Oligonucleotide-directed mutagenesis: a simple method using two oligonucleotide primers and a single-stranded DNA template, Methods in Enzymol. 154:329-350 (1987). Additional details on many of the above methods can be found in Methods in Enzymology Volume 154, which also describes useful controls for trouble-shooting problems with various mutagenesis methods.

Antibodies

In another aspect, antibodies to Orai and Stim polypeptides (or complexes thereof) can be generated using methods that are well known. The antibodies can be utilized for detecting and/or purifying polypeptides or complexes of interest, e.g., in situ to monitor localization of CRAC channel components, or simply in a biological sample of interest. Antibodies can optionally discriminate the polypeptides from homologues, and/or can be used in biosensor applications. Antibodies can also be used to block function of the polypeptides and complexes, in vivo, in situ or in vitro. Thus, antibodies to Orai and/or Stim and or a complex thereof can be used as therapeutic reagents. As used herein, the term “antibody” includes, but is not limited to, polyclonal antibodies, monoclonal antibodies, humanized or chimeric antibodies and biologically functional antibody fragments, which are those fragments sufficient for binding of the antibody fragment to the protein.

For the production of antibodies to a polypeptide encoded by one of the disclosed sequences or conservative variant or fragment thereof, various host animals may be immunized by injection with the polypeptide, or a portion thereof. Such host animals may include, but are not limited to, rabbits, mice and rats, to name but a few. Various adjuvants may be used to enhance the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals, such as those described above, may be immunized by injection with the encoded protein, or a portion thereof, supplemented with adjuvants as also described above.

Monoclonal antibodies (mAbs), which are homogeneous populations of antibodies to a particular antigen, may be obtained by any technique which provides for the production of antibody molecules by continuous cell lines in culture. These include, but are not limited to, the hybridoma technique of Kohler and Milstein (Nature 256:495-497, 1975; and U.S. Pat. No. 4,376,110), the human B-cell hybridoma technique (Kosbor et al., Immunology Today 4:72, 1983; Cole et al., Proc. Nat'l. Acad. Sci. USA 80:2026-2030, 1983), and the EBV-hybridoma technique (Cole et al., Monoclonal Antibodies and Cancer Therapy, Alan R. Liss, Inc., pp. 77-96, 1985). Such antibodies may be of any immunoglobulin class, including IgG, IgM, IgE, IgA, IgD, and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., Proc. Nat'l. Acad. Sci. USA 81:6851-6855, 1984; Neuberger et al., Nature 312:604-608, 1984; Takeda et al., Nature 314:452-454, 1985) by splicing the genes from a mouse antibody molecule of appropriate antigen specificity, together with genes from a human antibody molecule of appropriate biological activity, can be used. A chimeric antibody is a molecule in which different portions are derived from different animal species, such as those having a variable or hypervariable region derived from a murine mAb and a human immunoglobulin constant region.

Alternatively, techniques described for the production of single-chain antibodies (U.S. Pat. No. 4,946,778; Bird, Science 242:423-426, 1988; Huston et al., Proc. Nat'l. Acad. Sci. USA 85:5879-5883, 1988; and Ward et al., Nature 334:544-546, 1989) can be adapted to produce differentially expressed gene-single chain antibodies. Single chain antibodies are formed by linking the heavy and light chain fragments of the Fv region via an amino acid bridge, resulting in a single-chain polypeptide.

Antibody fragments which recognize specific epitopes may be generated by known techniques. For example, such fragments include, but are not limited to, the F(ab′)₂ fragments, which can be produced by pepsin digestion of the antibody molecule, and the Fab fragments, which can be generated by reducing the disulfide bridges of the F(ab′)₂ fragments. Alternatively, Fab expression libraries may be constructed (Huse et al., Science 246:1275-1281, 1989) to allow rapid and easy identification of monoclonal Fab fragments with the desired specificity.

The protocols for detecting and measuring the expression of the described polypeptides herein, using the above mentioned antibodies, are well known in the art. Such methods include, but are not limited to, dot blotting, western blotting, competitive and noncompetitive protein binding assays, enzyme-linked immunosorbant assays (ELISA), immunohistochemistry, fluorescence-activated cell sorting (FACS), and others commonly used and widely described in scientific and patent literature, and many employed commercially.

One method, for ease of detection, is the sandwich ELISA, of which a number of variations exist, all of which are intended to be encompassed by the present invention. For example, in a typical forward assay, unlabeled antibody is immobilized on a solid substrate and the sample to be tested is brought into contact with the bound molecule and incubated for a period of time sufficient to allow formation of an antibody-antigen binary complex. At this point, a second antibody, labeled with a reporter molecule capable of inducing a detectable signal, is then added and incubated, allowing time sufficient for the formation of a ternary complex of antibody-antigen-labeled antibody. Any unreacted material is washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay, in which the labeled antibody and sample to be tested are first combined, incubated and added to the unlabeled surface bound antibody. These techniques are well known to those skilled in the art, and the possibility of minor variations will be readily apparent. As used herein, “sandwich assay” is intended to encompass all variations on the basic two-site technique. For the immunoassays of the present invention, the only limiting factor is that the labeled antibody be an antibody which is specific for the protein expressed by the gene of interest.

The most commonly used reporter molecules in this type of assay are either enzymes, fluorophore- or radionuclide-containing molecules. In the case of an enzyme immunoassay, an enzyme is conjugated to the second antibody, usually by means of glutaraldehyde or periodate. As will be readily recognized, however, a wide variety of different ligation techniques exist which are well-known to the skilled artisan. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product, rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of PLAB which is present in the serum sample.

Alternately, fluorescent compounds, such as fluorescein and rhodamine, can be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

Antibodies specific for Orai, Stim or Orai/Stim complexes are useful in modulating (e.g., blocking) CRAC channel activation, as well as in targeting cells that express Orai and/or Stim. In human therapeutic applications of such antibodies, e.g., where modulation of CRAC channel activation is desired, including any of those applications noted herein, antibodies will normally be humanized before use. Thus, antibodies to Orai, Stim, or Orai/Stim can be generated by any available method as noted above, and subsequently humanized appropriately for use in vivo in humans. Many methods of humanizing antibodies are currently available, including those described in Howard and Kaser Making and Using Antibodies: A Practical Handbook ISBN: 0849335280 (2006). In typical approaches, humanized Abs are created by combining, at the genetic level, the complementarity-determining regions of a murine (or other mammalian) mAb with the framework sequences of a human Ab variable domain. This leads to a functional Ab with reduced immunogenic side effects in human therapy. Such techniques are generally described in U.S. Pat. Nos. 5,932,448; 5,693,762; 5,693,761; 5,585,089; 5,530,101; 5,569,825; 5,625,126; 5,633,425; 5,789,650; 5,661,016; and 5,770,429. Methods of making “superhumanized” antibodies with still further reduced immunogenicity in humans are described in Tan et al. (2002) ““Superhumanized” Antibodies: Reduction of Immunogenic Potential by Complementarity-Determining Region Grafting with Human Germline Sequences: Application to an Anti-CD28,” The Journal of Immunology, 169:1119-1125. Any available humanization method can be applied to making humanized antibodies of the present invention.

Regulating Expression of ORAI/STIM

Expression (e.g., transcription and/or translation) of orai or stim can be regulated using any of a variety of techniques known in the art. For example, gene expression can be inhibited using an antisense nucleic acid or an interfering RNA. Inhibition of expression in particular cell-types can be used for further studying the in vitro or in vivo role of these genes, and/or as a mechanism for treating a condition caused by overexpression of a orai or stim gene, and/or for treating a dominant effect caused by a particular allele of such a gene.

For example, use of antisense nucleic acids is well known in the art. An antisense nucleic acid has a region of complementarity to a target nucleic acid, e.g., a target gene, mRNA, or cDNA. Typically, a nucleic acid comprising a nucleotide sequence in a complementary, antisense orientation with respect to a coding (sense) sequence of an endogenous gene is introduced into a cell. The antisense nucleic acid can be RNA, DNA, a PNA or any other appropriate molecule. A duplex can form between the antisense sequence and its complementary sense sequence, resulting in inactivation of the gene. The antisense nucleic acid can inhibit gene expression by forming a duplex with an RNA transcribed from the gene, by forming a triplex with duplex DNA, etc. An antisense nucleic acid can be produced, e.g., for any gene whose coding sequence is known or can be determined by a number of well-established techniques (e.g., chemical synthesis of an antisense RNA or oligonucleotide (optionally including modified nucleotides and/or linkages that increase resistance to degradation or improve cellular uptake) or in vitro transcription). Antisense nucleic acids and their use are described, e.g., in U.S. Pat. No. 6,242,258 to Haselton and Alexander (Jun. 5, 2001) entitled “Methods for the selective regulation of DNA and RNA transcription and translation by photoactivation”; U.S. Pat. No. 6,500,615; U.S. Pat. No. 6,498,035; U.S. Pat. No. 6,395,544; U.S. Pat. No. 5,563,050; E. Schuch et al (1991) Symp Soc. Exp Biol 45:117-127; de Lange et al., (1995) Curr Top Microbiol Immunol 197:57-75; Hamilton et al. (1995) Curr Top Microbiol Immunol 197:77-89; Finnegan et al., (1996) Proc Natl Acad Sci USA 93:8449-8454; Uhlmann and A. Pepan (1990), Chem. Rev. 90:543; P. D. Cook (1991), Anti-Cancer Drug Design 6:585; J. Goodchild, Bioconjugate Chem. 1 (1990) 165; and, S. L. Beaucage and R. P. Iyer (1993), Tetrahedron 49:6123; and F. Eckstein, Ed. (1991), Oligonucleotides and Analogues—A Practical Approach, IRL Press.

Gene expression can also be inhibited by RNA silencing or interference. “RNA silencing” refers to any mechanism through which the presence of a single-stranded or, typically, a double-stranded RNA in a cell results in inhibition of expression of a target gene comprising a sequence identical or nearly identical to that of the RNA, including, but not limited to, RNA interference, repression of translation of a target mRNA transcribed from the target gene without alteration of the mRNA's stability, and transcriptional silencing (e.g., histone acetylation and heterochromatin formation leading to inhibition of transcription of the target mRNA).

The term “RNA interference” (“RNAi,” sometimes called RNA-mediated interference, post-transcriptional gene silencing, or quelling) refers to a phenomenon in which the presence of RNA, typically double-stranded RNA, in a cell results in inhibition of expression of a gene comprising a sequence identical, or nearly identical, to that of the double-stranded RNA. The double-stranded RNA responsible for inducing RNAi is called an “interfering RNA.” Expression of the gene is inhibited by the mechanism of RNAi as described below, in which the presence of the interfering RNA results in degradation of mRNA transcribed from the gene and thus in decreased levels of the mRNA and any encoded protein.

The mechanism of RNAi has been and is being extensively investigated in a number of eukaryotic organisms and cell types. See, for example, the following reviews: McManus and Sharp (2002) “Gene silencing in mammals by small interfering RNAs” Nature Reviews Genetics 3:737-747; Hutvagner and Zamore (2002) “RNAi: Nature abhors a double strand” Curr Opin Genet & Dev 200:225-232; Hannon (2002) “RNA interference” Nature 418:244-251; Agami (2002) “RNAi and related mechanisms and their potential use for therapy” Curr Opin Chem Biol 6:829-834; Tuschl and Borkhardt (2002) “Small interfering RNAs: A revolutionary tool for the analysis of gene function and gene therapy” Molecular Interventions 2:158-167; Nishikura (2001) “A short primer on RNAi: RNA-directed RNA polymerase acts as a key catalyst” Cell 107:415-418; and Zamore (2001) “RNA interference: Listening to the sound of silence” Nature Structural Biology 8:746-750. RNAi is also described in the patent literature; see, e.g., CA 2359180 by Kreutzer and Limmer entitled “Method and medicament for inhibiting the expression of a given gene”; WO 01/68836 by Beach et al. entitled “Methods and compositions for RNA interference”; WO 01/70949 by Graham et al. entitled “Genetic silencing”; and WO 01/75164 by Tuschl et al. entitled “RNA sequence-specific mediators of RNA interference.”

In brief, double-stranded RNA introduced into a cell (e.g., into the cytoplasm) is processed, for example by an RNAse III-like enzyme called Dicer, into shorter double-stranded fragments called small interfering RNAs (siRNAs, also called short interfering RNAs). The length and nature of the siRNAs produced is dependent on the species of the cell, although typically siRNAs are 21-25 nucleotides long (e.g., an siRNA may have a 19 base pair duplex portion with two nucleotide 3′ overhangs at each end). Similar siRNAs can be produced in vitro (e.g., by chemical synthesis or in vitro transcription) and introduced into the cell to induce RNAi. The siRNA becomes associated with an RNA-induced silencing complex (RISC). Separation of the sense and antisense strands of the siRNA, and interaction of the siRNA antisense strand with its target mRNA through complementary base-pairing interactions, optionally occurs. Finally, the mRNA is cleaved and degraded.

Expression of a target gene in a cell can thus be specifically inhibited by introducing an appropriately chosen double-stranded RNA into the cell. Guidelines for design of suitable interfering RNAs are known to those of skill in the art. For example, interfering RNAs are typically designed against exon sequences, rather than introns or untranslated regions. Characteristics of high efficiency interfering RNAs may vary by cell type. For example, although siRNAs may require 3′ overhangs and 5′ phosphates for most efficient induction of RNAi in Drosophila cells, in mammalian cells blunt ended siRNAs and/or RNAs lacking 5′ phosphates can induce RNAi as effectively as siRNAs with 3′ overhangs and/or 5′ phosphates (see, e.g., Czaudema et al. (2003) “Structural variations and stabilizing modifications of synthetic siRNAs in mammalian cells” Nucl Acids Res 31:2705-2716). As another example, since double-stranded RNAs greater than 30-80 base pairs long activate the antiviral interferon response in mammalian cells and result in non-specific silencing, interfering RNAs for use in mammalian cells are typically less than 30 base pairs (for example, Caplen et al. (2001) “Specific inhibition of gene expression by small double-stranded RNAs in invertebrate and vertebrate systems” Proc. Natl. Acad. Sci. USA 98:9742-9747, Elbashir et al. (2001) “Duplexes of 21-nucleotide RNAs mediate RNA interference in cultured mammalian cells” Nature 411:494-498 and Elbashir et al. (2002) “Analysis of gene function in somatic mammalian cells using small interfering RNAs” Methods 26:199-213 describe the use of 21 nucleotide siRNAs to specifically inhibit gene expression in mammalian cell lines, and Kim et al. (2005) “Synthetic dsRNA Dicer substrates enhance RNAi potency and efficacy” Nature Biotechnology 23:222-226 describes use of 25-30 nucleotide duplexes). The sense and antisense strands of a siRNA are typically, but not necessarily, completely complementary to each other over the double-stranded region of the siRNA (excluding any overhangs). The antisense strand is typically completely complementary to the target mRNA over the same region, although some nucleotide substitutions can be tolerated (e.g., a one or two nucleotide mismatch between the antisense strand and the mRNA can still result in RNAi, although at reduced efficiency). The ends of the double-stranded region are typically more tolerant to substitution than the middle; for example, as little as 15 bp (base pairs) of complementarity between the antisense strand and the target mRNA in the context of a 21 mer with a 19 bp double-stranded region has been shown to result in a functional siRNA (see, e.g., Czauderna et al. (2003) “Structural variations and stabilizing modifications of synthetic siRNAs in mammalian cells” Nucl Acids Res 31:2705-2716). Any overhangs can but need not be complementary to the target mRNA; for example, TT (two 2′-deoxythymidines) overhangs are frequently used to reduce synthesis costs.

Although double-stranded RNAs (e.g., double-stranded siRNAs) were initially thought to be required to initiate RNAi, several recent reports indicate that the antisense strand of such siRNAs is sufficient to initiate RNAi. Single-stranded antisense siRNAs can initiate RNAi through the same pathway as double-stranded siRNAs (as evidenced, for example, by the appearance of specific mRNA endonucleolytic cleavage fragments). As for double-stranded interfering RNAs, characteristics of high-efficiency single-stranded siRNAs may vary by cell type (e.g., a 5′ phosphate may be required on the antisense strand for efficient induction of RNAi in some cell types, while a free 5′ hydroxyl is sufficient in other cell types capable of phosphorylating the hydroxyl). See, e.g., Martinez et al. (2002) “Single-stranded antisense siRNAs guide target RNA cleavage in RNAi” Cell 110:563-574; Amarzguioui et al. (2003) “Tolerance for mutations and chemical modifications in a siRNA” Nucl. Acids Res. 31:589-595; Holen et al. (2003) “Similar behavior of single-strand and double-strand siRNAs suggests that they act through a common RNAi pathway” Nucl. Acids Res. 31:2401-2407; and Schwarz et al. (2002) Mol. Cell. 10:537-548.

Due to currently unexplained differences in efficiency between siRNAs corresponding to different regions of a given target mRNA, several siRNAs are typically designed and tested against the target mRNA to determine which siRNA is most effective. Interfering RNAs can also be produced as small hairpin RNAs (shRNAs, also called short hairpin RNAs), which are processed in the cell into siRNA-like molecules that initiate RNAi (see, e.g., Siolas et al. (2005) “Synthetic shRNAs as potent RNAi triggers” Nature Biotechnology 23:227-231).

The presence of RNA, particularly double-stranded RNA, in a cell can result in inhibition of expression of a gene comprising a sequence identical or nearly identical to that of the RNA through mechanisms other than RNAi. For example, double-stranded RNAs that are partially complementary to a target mRNA can repress translation of the mRNA without affecting its stability. As another example, double-stranded RNAs can induce histone methylation and heterochromatin formation, leading to transcriptional silencing of a gene comprising a sequence identical or nearly identical to that of the RNA (see, e.g., Schramke and Allshire (2003) “Hairpin RNAs and retrotransposon LTRs effect RNAi and chromatin-based gene silencing” Science 301:1069-1074; Kawasaki and Taira (2004) “Induction of DNA methylation and gene silencing by short interfering RNAs in human cells” Nature 431:211-217; and Morris et al. (2004) “Small interfering RNA-induced transcriptional gene silencing in human cells” Science 305:1289-1292).

Short RNAs called microRNAs (miRNAs) have been identified in a variety of species. Typically, these endogenous RNAs are each transcribed as a long RNA and then processed to a pre-miRNA of approximately 60-75 nucleotides that forms an imperfect hairpin (stem-loop) structure. The pre-miRNA is typically then cleaved, e.g., by Dicer, to form the mature miRNA. Mature miRNAs are typically approximately 21-25 nucleotides in length, but can vary, e.g., from about 14 to about 25 or more nucleotides. Some, though not all, miRNAs have been shown to inhibit translation of mRNAs bearing partially complementary sequences. Such miRNAs contain one or more internal mismatches to the corresponding mRNA that are predicted to result in a bulge in the center of the duplex formed by the binding of the miRNA antisense strand to the mRNA. The miRNA typically forms approximately 14-17 Watson-Crick base pairs with the mRNA; additional wobble base pairs can also be formed. In addition, short synthetic double-stranded RNAs (e.g., similar to siRNAs) containing central mismatches to the corresponding mRNA have been shown to repress translation (but not initiate degradation) of the mRNA. See, for example, Zeng et al. (2003) “MicroRNAs and small interfering RNAs can inhibit mRNA expression by similar mechanisms” Proc. Natl. Acad. Sci. USA 100:9779-9784; Doench et al. (2003) “siRNAs can function as miRNAs” Genes & Dev. 17:438-442; Bartel and Bartel (2003) “MicroRNAs: At the root of plant development?” Plant Physiology 132:709-717; Schwarz and Zamore (2002) “Why do miRNAs live in the miRNP?” Genes & Dev. 16:1025-1031; Tang et al. (2003) “A biochemical framework for RNA silencing in plants” Genes & Dev. 17:49-63; Meister et al. (2004) “Sequence-specific inhibition of microRNA- and siRNA-induced RNA silencing” RNA 10:544-550; Nelson et al. (2003) “The microRNA world: Small is mighty” Trends Biochem. Sci. 28:534-540; Scacheri et al. (2004) “Short interfering RNAs can induce unexpected and divergent changes in the levels of untargeted proteins in mammalian cells” Proc. Natl. Acad. Sci. USA 101:1892-1897; Sempere et al. (2004) “Expression profiling of mammalian microRNAs uncovers a subset of brain-expressed microRNAs with possible roles in murine and human neuronal differentiation” Genome Biology 5:R13; Dykxhoorn et al. (2003) “Killing the messenger: Short RNAs that silence gene expression” Nature Reviews Molec. and Cell Biol. 4:457-467; McManus (2003) “MicroRNAs and cancer” Semin Cancer Biol. 13:253-288; and Stark et al. (2003) “Identification of Drosophila microRNA targets” PLoS Biol. 1:E60.

The cellular machinery involved in translational repression of mRNAs by partially complementary RNAs (e.g., certain miRNAs) appears to partially overlap that involved in RNAi, although, as noted, translation of the mRNAs, not their stability, is affected and the mRNAs are typically not degraded.

The location and/or size of the bulge(s) formed when the antisense strand of the RNA binds the mRNA can affect the ability of the RNA to repress translation of the mRNA. Similarly, location and/or size of any bulges within the RNA itself can also affect efficiency of translational repression. See, e.g., the references above. Typically, translational repression is most effective when the antisense strand of the RNA is complementary to the 3′ untranslated region (3′ UTR) of the mRNA. Multiple repeats, e.g., tandem repeats, of the sequence complementary to the antisense strand of the RNA can also provide more effective translational repression; for example, some mRNAs that are translationally repressed by endogenous miRNAs contain 7-8 repeats of the miRNA binding sequence at their 3′ UTRs. It is worth noting that translational repression appears to be more dependent on concentration of the RNA than RNA interference does; translational repression is thought to involve binding of a single mRNA by each repressing RNA, while RNAi is thought to involve cleavage of multiple copies of the mRNA by a single siRNA-RISC complex.

Guidance for design of a suitable RNA to repress translation of a given target mRNA can be found in the literature (e.g., the references above and Doench and Sharp (2004) “Specificity of microRNA target selection in translational repression” Genes & Dev. 18:504-511; Rehmsmeier et al. (2004) “Fast and effective prediction of microRNA/target duplexes” RNA 10:1507-1517; Robins et al. (2005) “Incorporating structure to predict microRNA targets” Proc Natl Acad Sci 102:4006-4009; and Mattick and Makunin (2005) “Small regulatory RNAs in mammals” Hum. Mol. Genet. 14:R121-R132, among many others) and herein. However, due to differences in efficiency of translational repression between RNAs of different structure (e.g., bulge size, sequence, and/or location) and RNAs corresponding to different regions of the target mRNA, several RNAs are optionally designed and tested against the target mRNA to determine which is most effective at repressing translation of the target mRNA.

Kits

In an additional aspect, the present invention provides kits embodying the methods, composition, systems or apparatus herein. Kits of the invention optionally comprise one or more of the following: (1) a composition, system, system component as described herein; (2) instructions for practicing the methods described herein, and/or for using the compositions or operating the system or system components herein; (3) one or more Orai or Stim polypeptide or coding nucleic acid; (4) a container for holding components or compositions, and, (5) packaging materials.

### EXAMPLES

### Example 1

### A Genome-Wide RNAi Screen of Ca²⁺ Influx Identifies Genes that Regulate CRAC Channel Activity

Recent studies by our group and others demonstrated a required and conserved role of Stim in store-operated Ca²⁺ (SOC) influx and Ca²⁺ release-activated Ca²⁺ (CRAC) channel activity. Using an unbiased genome-wide RNAi screen in Drosophila S2 cells, we identified 75 hits that strongly inhibited Ca²⁺ influx upon store emptying by thapsigargin (TG). Among these hits are 11 predicted transmembrane proteins, including Stim and one, olf186-F, that upon RNAi-mediated knockdown exhibited a profound reduction of TG-evoked Ca²⁺ entry and CRAC current, and upon overexpression a three-fold augmentation of CRAC current. CRAC currents were further increased to eight-fold higher than control and developed more rapidly when olf186-F was co-transfected with Stim. olf186-F is a member of a highly conserved family of four-transmembrane spanning proteins with homologs from C. elegans to human. The ER Ca²⁺ pump SERCA and the SNARE protein Syntaxin5 were also required for CRAC channel activity, consistent with a signaling pathway in which Stim senses Ca²⁺ depletion within the ER, translocates to the plasma membrane, and interacts with olf186-F to trigger CRAC channel activity.

Patch-clamp experiments have identified the biophysical characteristics of Ca²⁺ release-activated Ca²⁺ (CRAC) channels in lymphocytes and other human cell types (1, 2). Despite the acknowledged functional importance of store-operated Ca²⁺ (SOC) influx in cell biology (2) and of CRAC channels for immune cell activation (3), the intrinsic channel components and signaling pathways that lead to channel activation remain unidentified. In previous work, we demonstrated that SOC influx in S2 cells occurs through a channel that shares biophysical properties with CRAC channels in human T lymphocytes (4). In a medium-throughput RNAi screen targeting 170 candidate genes in S2 cells, we discovered an essential conserved role of Stim and the mammalian homolog STIM1 in SOC influx and CRAC channel activity (5). STIM1 and STIM2 were also identified in an independently performed screen of HeLa cells using the Drosophila enzyme Dicer to generate small interfering RNA species from dsRNA (6). Drosophila Stim and the mammalian homolog STIM1 appear to play dual roles in the CRAC channel activation sequence, sensing the luminal Ca²⁺ store content through an EF hand motif and trafficking from an ER-like localization to the plasma membrane to trigger CRAC channel activity (6-8). However, as single-pass transmembrane proteins, Stim and its mammalian homolog STIM1 are unlikely to form the CRAC channel itself. To search systematically for additional components of the CRAC channel, and to analyze the signaling network and other required factors that lead to store-operated Ca²⁺ channel activity, we devised and performed a genome-wide screen on S2 cells based upon a fluorescence assay of Ca²⁺ influx. The library at Harvard's Drosophila RNAi Screening Center (DRSC) of 23845 double-stranded RNAs (dsRNA amplicons) has been used in several previous functional screens (9-14).

A recent report identified a genetic defect in patients with severe combined immune deficiency (SCID) (15). The screen in this study made use of the ability of thapsigargin (TG) to send NFAT-GFP to the nucleus in S2 cells, providing an assay for disruption of signaling anywhere in the cascade from elevated [Ca²⁺]ᵢ to calcineurin activation and nuclear relocalization of NFAT. The fly gene olf186-F (named Orai) was identified in the screen, and a human homolog on chromosome 12 was shown to be mutated in SCID patients, resulting in the loss of CRAC channel activity. Heterologous expression of the wild-type human homolog, that was named Orai1, restored CRAC channel activity in SCID T cell lines.

Here, based upon direct Ca²⁺ influx measurements in a genome-wide screen, we identify several genes that are required for CRAC channel function in S2 cells. Our results confirm the functional requirement of olf186-F (Orai) for Ca²⁺ signaling and extend these results to investigate effects of knockdown and overexpression on CRAC channel activity. We also show that the sarco-/endoplasmic reticulum calcium ATPase (SERCA) pump and the trafficking protein Syntaxin 5 are required for CRAC channel activity.

Cell culture and transfection. Drosophila S2 cells (Invitrogen) used in the RNAi screen, single cell imaging and patch-clamp experiments were propagated in Schneider's medium (Invitrogen) supplemented with 10% FBS (Invitrogen) at 24° C. Cells were seeded at a density of 10⁶ cells/ml and passaged when the cells achieved a density of ˜6×10⁶ cells/ml. S2 cells were transfected (see clones described later) using a Nucleofector (Amaxa) following the manufacturer's protocol. Forty-eight hours posttransfection, cells were used for patch-clamp experiments or processed for RT-PCR analysis. The library at Harvard's Drosophila RNAi Screening Center (DRSC) of 23845 double-stranded RNAs (dsRNA amplicons) has been used in several previous functional screens.

Molecular cloning. A cDNA clone, pAc5.1/olf186-F, encoding full-length Drosophila olf186-F-RB was generated for transfection into S2 cells. Briefly, a 1.1 kb fragment was isolated from total mRNA of Drosophila S2 cells by RT-PCR, and subcloned between the XhoI and NotI sites of pAc5.1/V5-His B expression vector. Primers were designed based on the deposited flybase sequence of olf186-F (CG11430RB). Resulting clones were confirmed by sequencing. Generation of pAc5.1/EGFP and pAc5.1/D-STIM were as described previously.

Preparation of Double-Stranded RNA (dsRNA) for Validation at Single cell level. PCR templates for dsRNA synthesis were either from the DRSC stock or RT-PCRed from cultured S2 cells (olf186-F). Primers were designed based on the original amplicon sequences to produce ˜500 bp fragments with T7 polymerase binding sites on both sense and anti-sense strands. For PCR primer pairs, see Table 4. The MEGAscript RNAi kits (Ambion) were used to synthesize the dsRNA as per manufacturer's protocol. The concentration of dsRNA was determined by optical density at 260 nm.

RNAi in Drosophila S2 cells. RNAi experiments were adapted from the protocols described by Worby et al. (2). Drosophila S2 cells (0.5×10⁶) were seeded in T-25 flasks in 2 ml of complete S2 media. The next day, media was removed and replaced with 2 ml serum-free S2 media. 20 μg dsRNA was added and cells were incubated at room temperate for 45 min with gentle rocking. 4 ml S2 media was added and cells were incubated for 5 days at 24° C. Cells were then harvested and either plated for single cell Ca²⁺ imaging and patch-clamp experiments or processed for RT-PCR analysis.

RNA isolation and RT-PCR. RNA was isolated using TRIZOL (Invitrogen) following the manufacturer's protocols. The total RNA yield was calculated from the OD₂₆₀ of the RNA preparation. RNA quality was determined from the absorbance ratio OD₂₆₀/OD₂₈₀ (>1.8). In each sample, total RNA (3 μg) was reverse-transcribed using the Superscript Preamplification System (Invitrogen). The sense and antisense primers were specifically designed from the coding regions of our targeted genes (Table 4). The fidelity and specificity of the sense and antisense oligonucleotides has been examined using the BLAST program. PCR reactions were performed by DNA thermal cycler (Bio-rad) using Platinum PCR Supermix High Fidelity (Invitrogen). The first-strand cDNA reaction mixture (1 μl) was used in a 50 μl PCR reaction consisting of 0.2 μM paired primers. The cDNA samples were amplified under the following conditions: the mixture was denatured at 94° C. (30 sec), annealed at 55° C. (30 sec), and extended at 68° C. (30 sec) for 25-27 cycles, followed by a final extension at 72° C. (10 min) to ensure complete product extension. The PCR products were electrophoresed through a 1.5% agarose gel and amplified cDNA bands were visualized by GelStar (Cambrex) staining.

Single-cell [Ca₂₊]ᵢ imaging. Ratiometric [Ca₂₊]ᵢ imaging was performed as described, using solution recipes described in Table 3. Transfected cells were recognized by co-expressed enhanced green fluorescent protein (EGFP), using filters to avoid contamination of Fura-2 fluorescence by bleed-through of GFP fluorescence. Data were analysed with Metafluor software (Universal Imaging) and OriginPro 7.5 software (OriginLab) and are expressed as means ±SEM.

Whole-cell recording. Patch-clamp experiments were performed at room temperature in the standard whole-cell recording configuration. The recipes of external and internal solutions are indicated in Table 3. The membrane capacitance (a measure of cell surface area) of S2 cells selected for recording was 9.15±0.27 pF (mean ±SEM, n=287 cells, 22 experiments). To calculate current densities, peak current amplitudes were divided by membrane capacitance for each cell.

Bioinformatics. Phi-blast server at NCBI was used to look for homologous proteins of the Drosophila olf186-F gene product. The criteria used were: E-value<1e−20 and the length of homology regions must be at least ⅔ of the full proteins. The sequences of all family members identified were clustered using clustalW and a phylogenetic tree (phylogram) was generated according to the mutual similarity among the members.

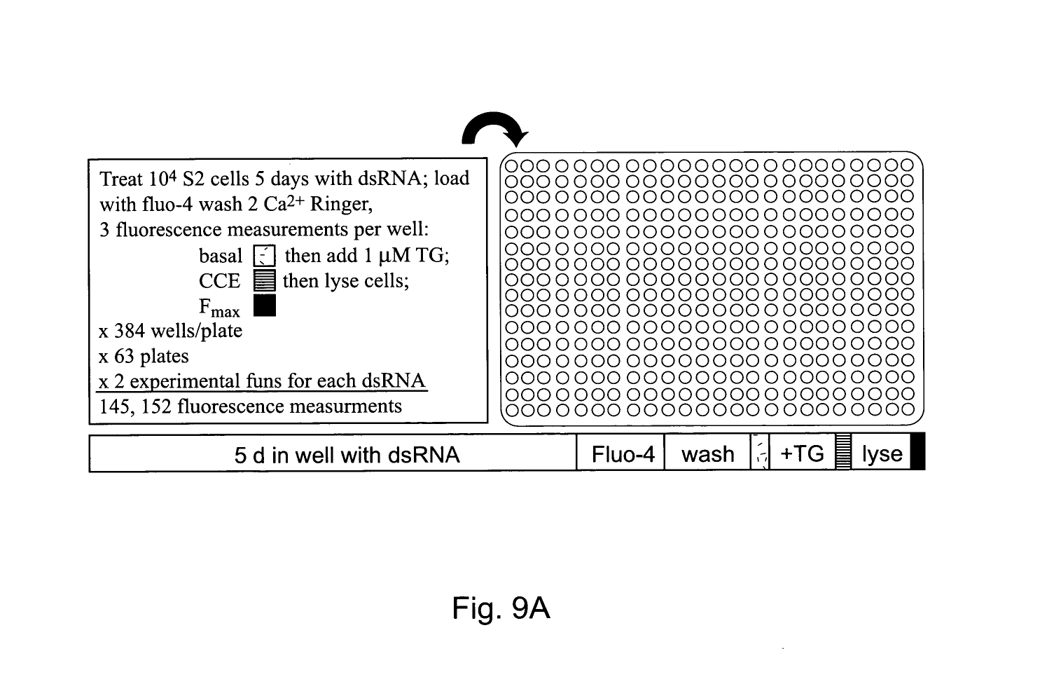

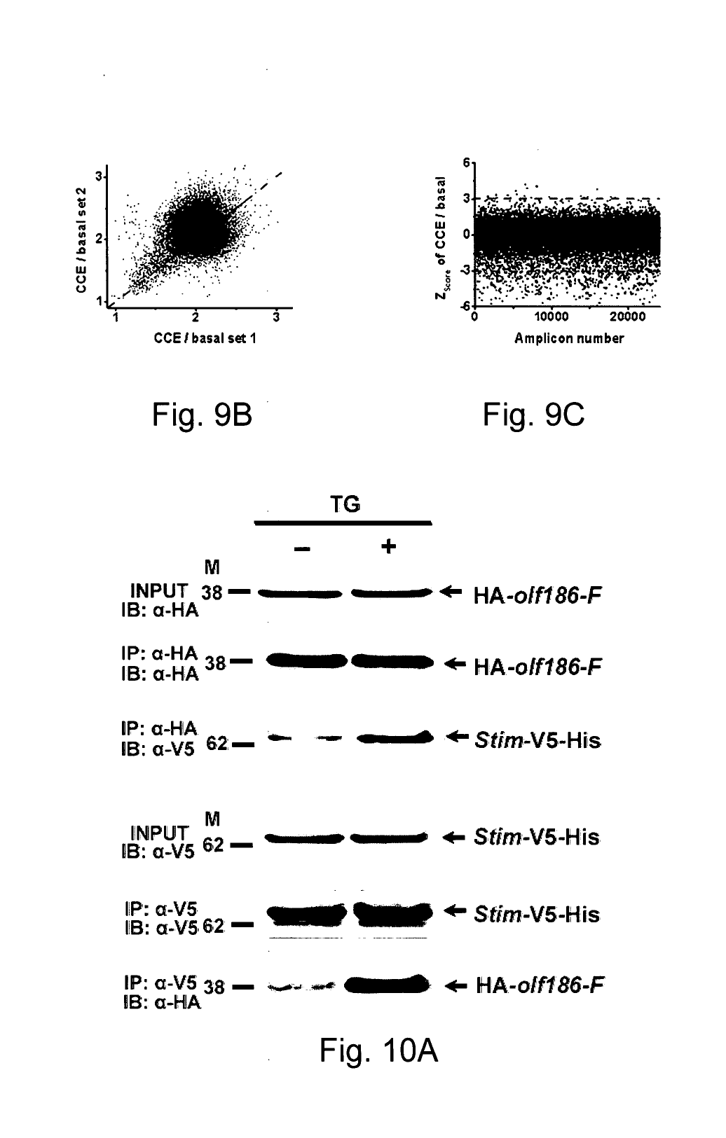

Genome-wide RNAi screen. Drosophila S2 cells were cultured in 384-well plates containing ˜0.25 μg dsRNA (˜10⁴ cells/well). Each plate included a well with dsRNA targeting Stim as a positive control. After 5 days, cells were loaded with a [Ca²⁺]ᵢ indicator fluo-4/AM (10 μM, Molecular Probes); free dye was then washed by Ringer solution containing 2 mM Ca²⁺ (see Table 3 for all solution recipes). Three fluorescence measurements were systematically performed: basal (resting intracellular free Ca²⁺); CCE (TG-dependent Ca²⁺ influx assessed 4 min after addition of TG) and Fₘₐₓ (maximal fluorescence 15 min after addition of triton X-100 to a final concentration ˜2% to detect changes in cell number). FIG. 9A illustrates a schematic diagram. Values of “basal/Fₘₐₓ” were calculated for each well to indicate the normalized resting [Ca²⁺]ᵢ level and values of “CCE/basal” were computed to represent the relative CCE levels. The screen was carried out in duplicate. To correct for variation in dye loading or cell number, ratios of fluorescence values (CCE/basal) as an index for Ca²⁺ influx evoked by TG were computed. A scatter plot showed reasonable agreement for the replicate assays for most amplicons (FIG. 9B), particularly for hits with reduced Ca²⁺ influx reflected in lower CCE/basal values. Because most amplicons did not influence the dynamics of Ca²⁺ signaling, the average for a given plate was very close to that of non-treated wells. Therefore, z-scores of “basal/Fₘₐₓ” and “CCE/basal” equal to the value of the well minus the average of the plate divided by the standard deviation for the plate were calculated for each well. The averaged z-scores (FIG. 9C) represent variations in the distribution of CCE/basal measurements for each amplicon. Hits in the screen, defined by values more than 3 standard deviations from the mean (Z-score<−3 or >3) fell into four categories: 1) decreased resting [Ca²⁺]ᵢ; 2) increased resting [Ca²⁺]ᵢ; 3) decreased CCE (Table 2); and 4) increased CCE. In order to eliminate false-positive outcomes, putative hits with a z-score of Fₘₐₓ smaller than −2, or with more than five off-targets, were generally filtered out from the lists. Overlapping hits between groups 1 and 4 and groups 2 and 3 were removed from group 4 and 3, respectively.

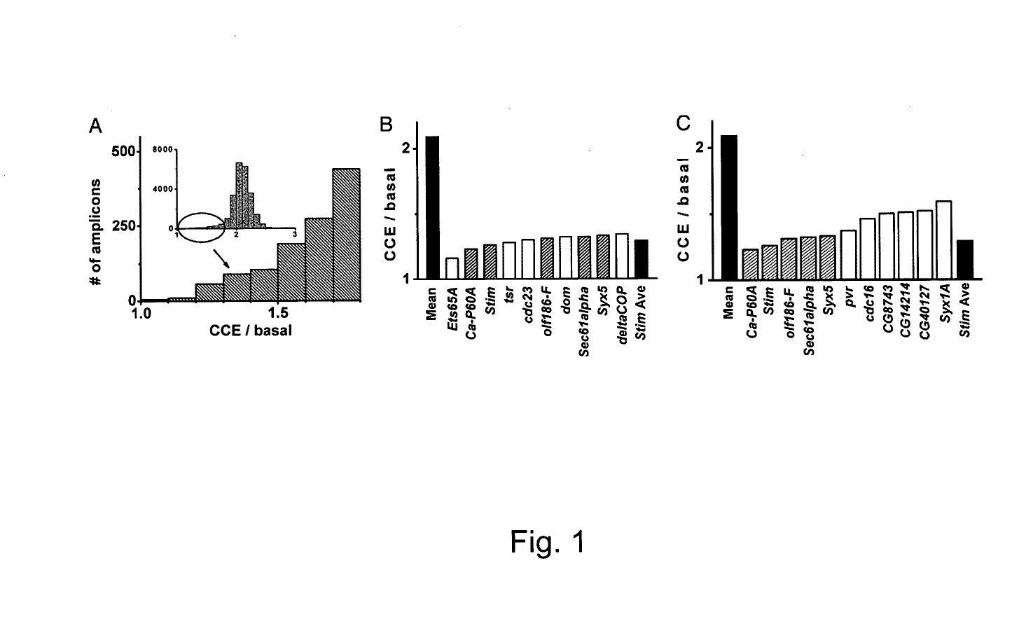

A genome-wide screen for SOC influx. Each well of 63 separate 384-well plates contained an individual dsRNA amplicon. Ca²⁺-indicator fluorescence measurements were made in each well to monitor cytosolic Ca²⁺ ([Ca²⁺]ᵢ) before (basal) and after (CCE) addition of TG. Thapsigargin inhibits SERCA pump-mediated reuptake of Ca²⁺ into cellular stores, depleting them and triggering capacitive calcium entry (CCE) in S2 cells as well as in mammalian cells. Hits in the screen were defined by significantly reduced CCE/basal values, as described in Methods and illustrated by a tail in the histogram shown in FIG. 1A. The “top 10 hits”, with strong suppressive effects comparable to the average value of the Stim positive control (CCE/basal ˜1.3), were selected for further evaluation (FIG. 1B; Table 1). Among the 75 filtered hits with z-scores of CCE/basal<−3 (Table 2), only 11 contained transmembrane segments, as illustrated in FIG. 1C. Among these, the five strongest hits are annotated in Flybase (http://flybase.org) as Ca-P60A, Stim, olf186-F, sec61alpha, and Syx5.

The consistent suppressive effect of Stim dsRNA validates the present screen. However, Stim is unlikely to constitute the CRAC channel, since multiple transmembrane segments are found in all known ion channel pore-forming subunits. The protein product of sec61alpha is a subunit of the translocon complex, which recognizes and delivers newly synthesized membrane proteins into ER, and may be a hit in this screen by altering synthesis or localization of other essential components. Ca-P60A is the SERCA pump gene in fly, whose products are located in the ER for filling/refilling the Ca²⁺ store. Syx5 generates a single transmembrane soluble NSF attachment receptor (SNARE) protein (Syntaxin 5) which is essential for vesicle fusion and may modulate CCE by altered protein trafficking rather than serving as the channel pore. Thus, among the top 10 hits, olf186-F is the only gene of unknown structure and function that is predicted to contain multiple transmembrane segments.

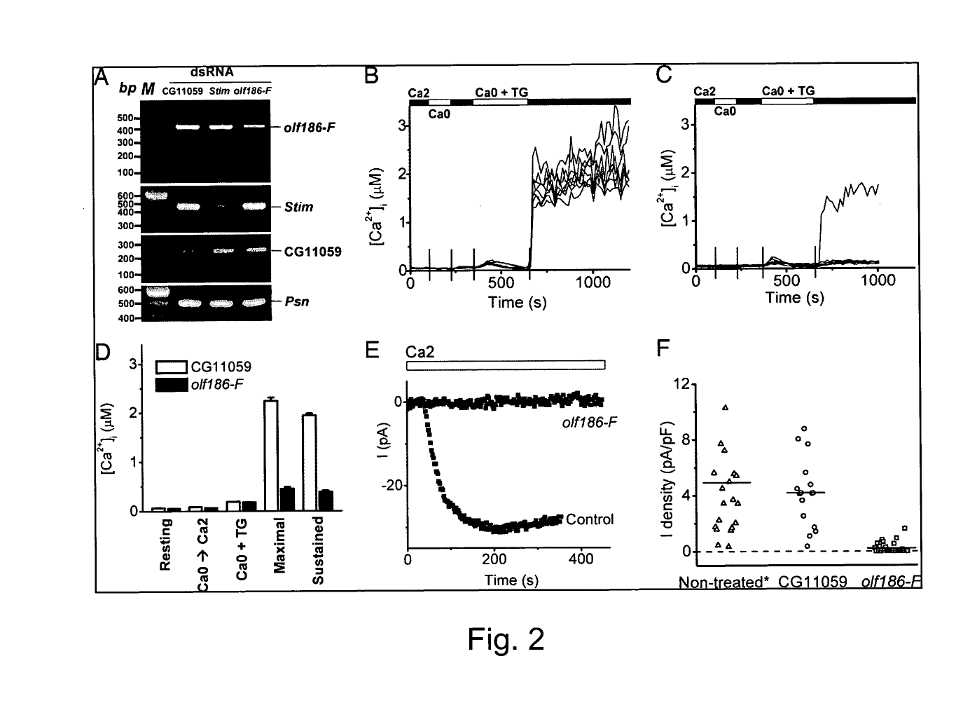

Effects of olf186-F knock-down and overexpression on Ca²⁺ influx and CRAC currents in single cells. To clarify effects of suppressing olf186-F at the level of single cells, Ca²⁺ signaling and CRAC currents in cells treated with dsRNA for olf186-F were examined in comparison with untreated cells or with cells treated with dsRNA for CG11059, an irrelevant cell adhesion molecule, as controls. RT-PCR showed more than 50% decrease of olf186-F mRNA expression, compared with controls (FIG. 2A). FIG. 2B illustrates ratiometric fura-2 [Ca²⁺]ᵢ measurements before and after TG-evoked store depletion in eight individual control cells. Addition of TG in zero-Ca²⁺ solution to deplete the store elicited a Ca²⁺ release transient caused by net leak of Ca²⁺ from the store when the reuptake pump is blocked. Upon readdition of external Ca²⁺, a robust Ca²⁺ signal was observed in every cell. In cells pre-treated with olf186-F dsRNA, neither the resting [Ca²⁺]ᵢ level nor the release transient were significantly altered, but the rise in [Ca²⁺]ᵢ upon readdition of external Ca²⁺ was strongly suppressed in the vast majority of the individual cells (FIG. 2C). FIG. 2D clearly demonstrates that suppression of olf186-F effectively inhibits both the early and sustained components of Ca²⁺ entry evoked by TG at the single-cell level. Comparable inhibition was obtained in cells pretreated with Stim dsRNA as a positive control (data not shown), consistent with our previous report.

Patch-clamp experiments confirmed a dramatic suppression of CRAC currents following knockdown of olf186-F (FIGS. 2E and 2F). CRAC current normally develops after establishing the whole-cell recording configuration as the cytoplasm is dialyzed by a pipette solution containing a strong Ca²⁺ chelator to reduce cytosolic [Ca²⁺]ᵢ and deplete internal stores. With this method of “passive stores depletion”, current increases after an initial delay to a maximum value before declining slowly. However, in the majority of cells pretreated with olf186-F dsRNA, CRAC current was completely suppressed, as illustrated by the representative traces in FIG. 2E and by a chart of CRAC current densities (FIG. 2F). As was previously demonstrated for Stim, olf186-F expression is required for normal CRAC channel activity.

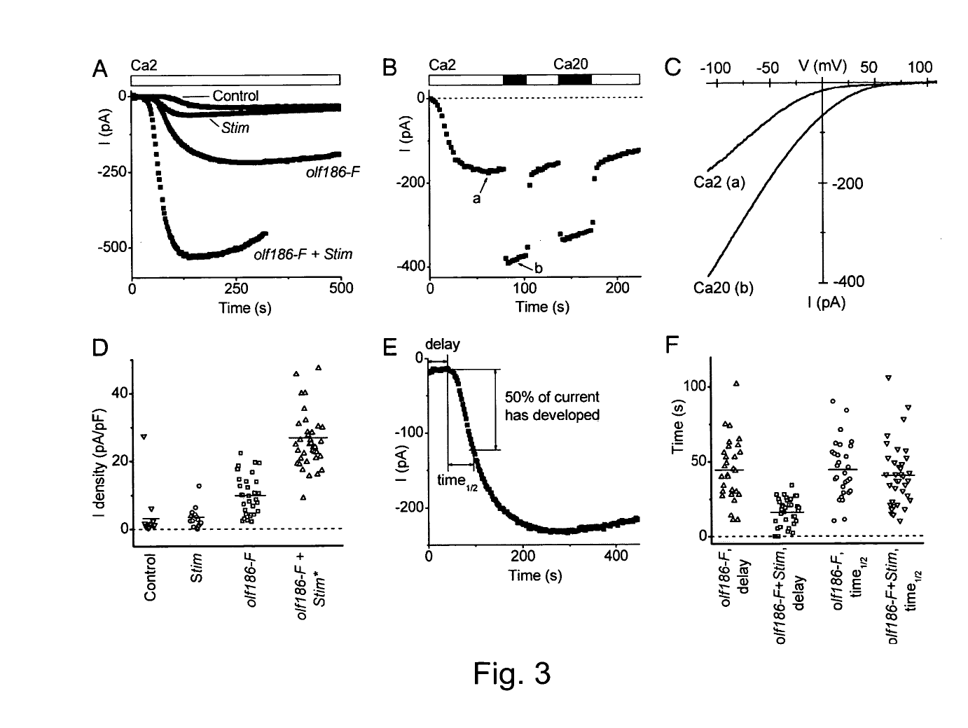

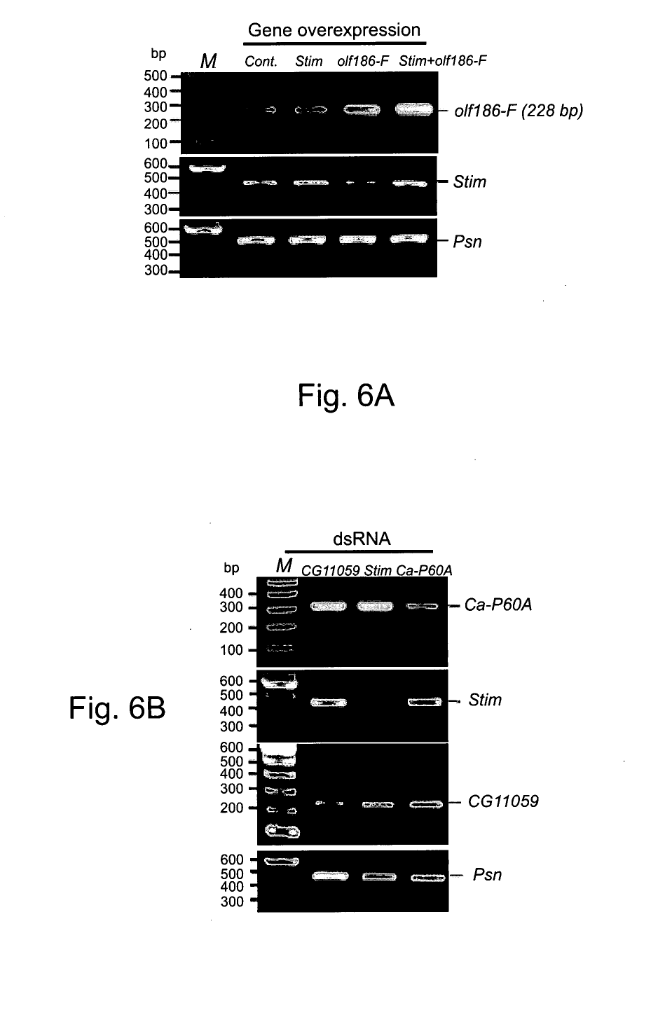

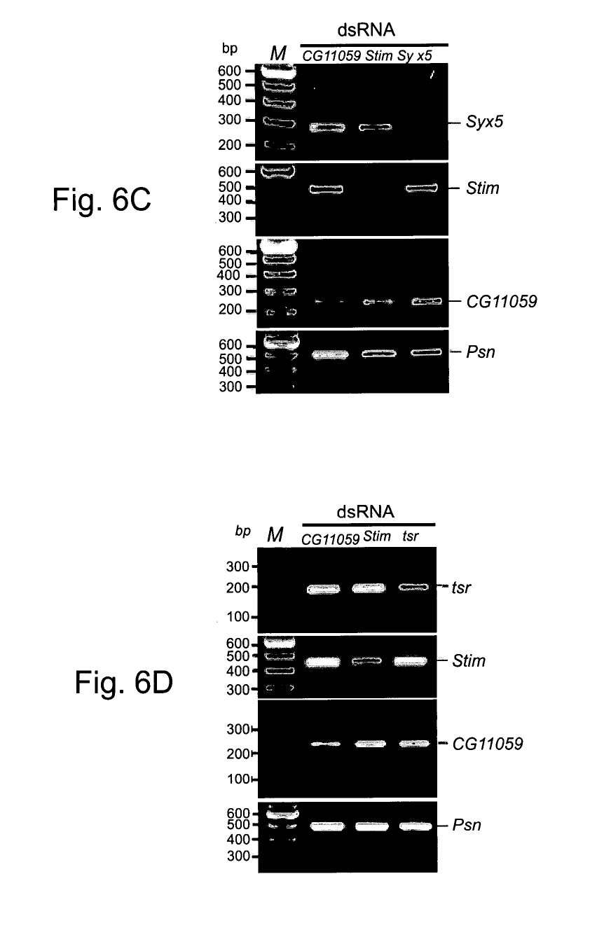

The function of olf186-F was examined further by cloning its full-length cDNA from S2 cells and inserting it into a Drosophila expression vector. The olf86-F clone was overexpressed with or without a co-transfected Stim clone in S2 cells, using a co-transfected GFP construct for identification of transfected cells. Increased expression levels of olf186-F and Stim following separate transfections or co-transfection were verified by RT-PCR (FIG. 6A). FIG. 3A illustrates the time course of current development following break-in to achieve whole-cell recording in four representative cells. Expression of Stim by itself had no significant effect on current amplitude compared to control, untransfected cells. However, when olf186-F was overexpressed CRAC current increased significantly; and when olf186-F was co-expressed with Stim CRAC current was further enhanced. The induced current following co-transfection of olf186-F with Stim exhibited Ca²⁺ selectivity and current-voltage shapes indistinguishable from native CRAC current (FIGS. 3B and 3C). When external Ca²⁺ was elevated ten-fold, the current magnitudes approximately doubled, as is the case for native CRAC current in S2 cells, and current-voltage curves had the same inwardly rectifying characteristic. FIG. 3D illustrates CRAC current densities for individual cells in each group of transfected cells. Overexpression of olf186-F increased the average current density three-fold; and although Stim by itself did not alter current density, co-transfection with olf186-F produced a remarkable eight-fold enhancement. Interestingly, co-transfection with Stim also decreased the initial delay to the onset of current development (FIGS. 3A, 3E, and 3F). Together, these results show that overexpression of olf186-F is sufficient to increase CRAC current density, that co-expression with Stim produces a further enhancement, and that interaction with Stim may be the rate-limiting step for channel activation.

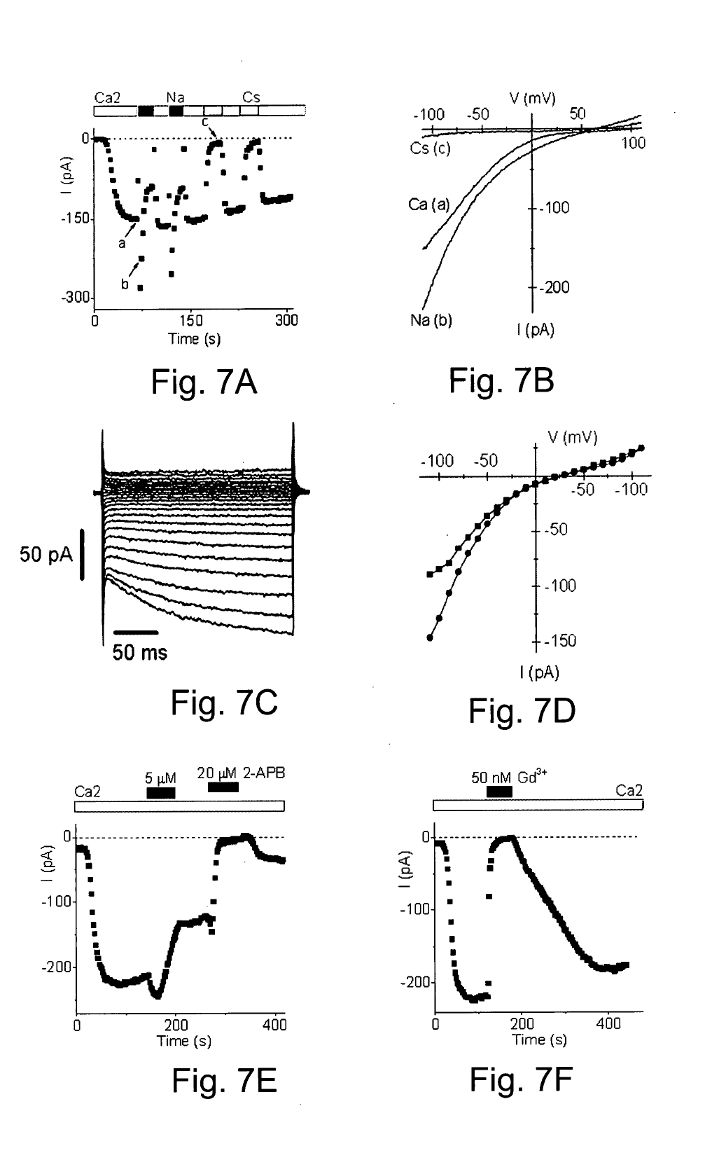

Apart from much larger current amplitudes, the Ca²⁺-selective current in cells co-transfected with olf186-F and Stim exhibited biophysical properties that were indistinguishable from native CRAC currents. Monovalent ion selectivity upon removal of external Ca²⁺ (divalent-free), Na⁺ current inactivation, and potentiation of Ca²⁺ current upon readdition of external Ca²⁺ were similar to that described for native CRAC current in lymphocytes and S2 cells (FIG. 7A). Current-voltage relations for the monovalent Na⁺ current showed inward rectification and a reversal potential of +65 mV (FIG. 7B), indicating a permeability ratio of Na⁺ to Cs⁺ (P_{Na}/P_{Cs}) of 0.076, the same as native monovalent CRAC current. The response to voltage steps was also the same, with currents that increase slightly at very negative potentials (FIGS. 7C and 7D), as seen previously in S2 cells. Furthermore, the Ca²⁺ current in olf186-F+Stim transfectants was sensitive to pharmacological agents that act on native CRAC currents (FIGS. 7E and 7F). Gd³⁺ (50 nM) and 2-aminoethyldiphenyl borate (2-APB; 20 μM) blocked the enhanced Ca²⁺ currents, and at lower concentration (5 μM) 2-APB exhibited a characteristic potentiation of current before blocking. In summary, the ion selectivity, development and inactivation kinetics, and pharmacological profile of the large induced Ca²⁺ current following overexpression of olf186-F plus Stim match native CRAC currents. Because the current is not enhanced by overexpression of Stim alone, these findings support the possibility that olf186-F itself is part of the channel.

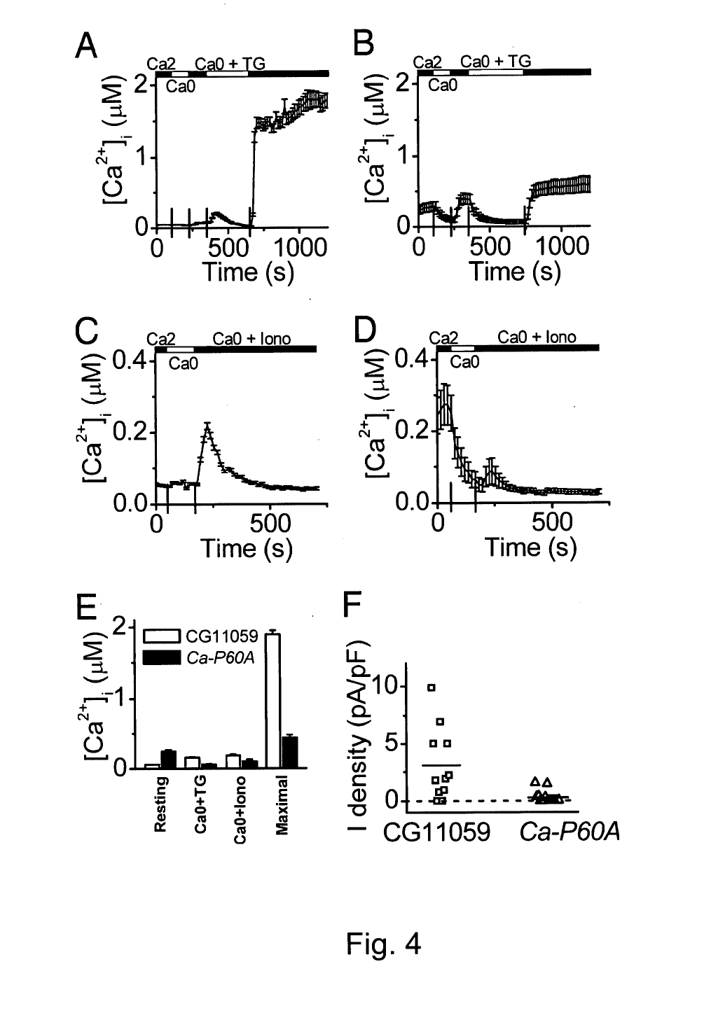

Effects of Ca-P60A, Syx5, and tsr dsRNA treatment on Ca²⁺ dynamics and CRAC current. The SERCA pump also emerged from the RNAi screen as a putative regulator of SOC influx. However, the screen was based on Ca²⁺ influx induced by TG (which blocks the SERCA pump), and thus the potential for a false-positive hit was of concern. Accordingly, single-cell Ca²⁺ imaging and patch-clamp experiments were performed using alternative stimuli (ionomycin, passive stores depletion) to deplete the Ca²⁺ store. Selective lowering of Ca-P60A mRNA was first verified by RT-PCR (FIG. 6B). Knockdown of Ca-P60A significantly increased resting [Ca²⁺]ᵢ, reduced the store release transient upon addition of TG, and strongly suppressed Ca²⁺ influx upon readdition of external Ca²⁺ (FIGS. 4A and 4B). In addition, ionomycin in zero-Ca²⁺ solution applied to control cells evoked a sharp Ca²⁺ release transient with a peak that averaged ˜200 nM, but a greatly reduced release transient in Ca-P60A dsRNA-treated cells (FIGS. 4C and 4D), indicating reduced Ca²⁺ store content as a consequence of reduced SERCA pump activity. As shown by the summary of Ca²⁺ imaging experiments (FIG. 4E), knockdown of SERCA has a strong Ca²⁺ phenotype, raising resting [Ca²⁺]ᵢ, reducing release transients, and suppressing influx evoked by TG. Furthermore, patch-clamp experiments demonstrated that CRAC currents were also suppressed when stores were depleted passively by dialysis of a Ca²⁺ chelator (FIG. 4F), confirming a requirement of Ca-P60A for activation of functional CRAC channels.

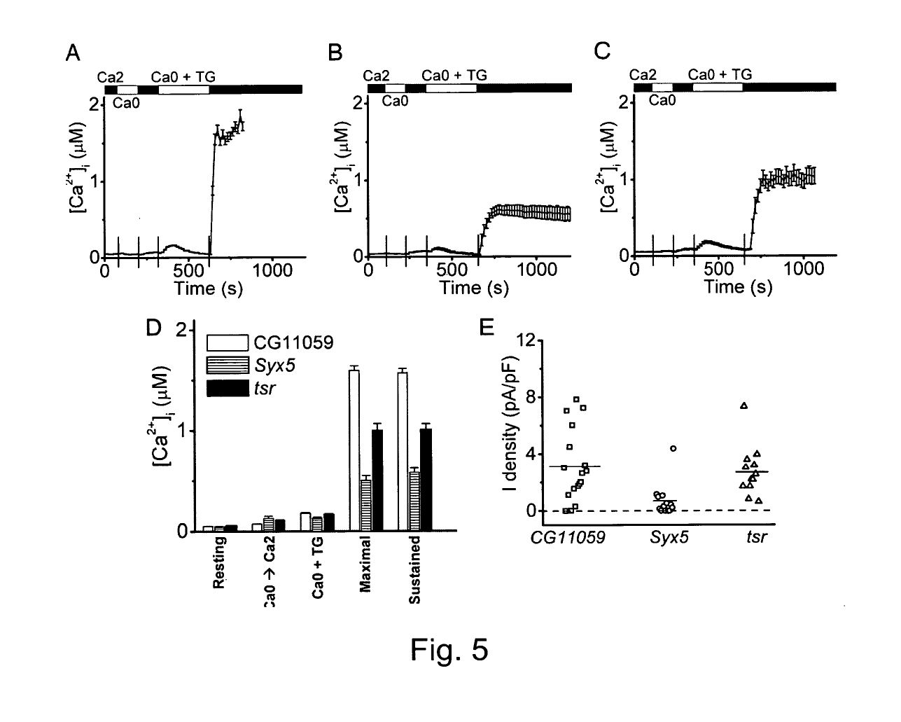

Several trafficking proteins were also identified as putative regulators of SOC activity (Table 2). Syx5 is a syntaxin, several of which have been implicated in SNARE complexes that regulate vesicle trafficking; and tsr is referred to as an actin-binding protein that regulates cytoskeleton remodeling. A putative role of its human homolog, cofilin, has been reported in activation of store-operated calcium entry in platelets. Both Syx5 and tsr dsRNA pre-incubation caused significant and selective lowering of mRNA levels (FIGS. 6C and 6D) and a corresponding inhibition of TG-dependent Ca²⁺ influx in S2 cells, without altering the resting [Ca²⁺]ᵢ or store release (compare FIGS. 5A-5C). FIG. 5D summarizes the inhibition of TG-evoked [Ca²⁺]ᵢ influx when Syx5 or tsr expression was knocked down. Patch-clamp experiments confirmed that CRAC currents were indeed suppressed during passive stores depletion when Syx5 was knocked down, but effects of tsr knockdown on CRAC currents did not achieve statistical significance (FIG. 5E).

TABLE 1B
TOP 10 hits involved in store-operated Ca²⁺ entry

| | | | | | | | number |
|---|---|---|---|---|---|---|---|
| | | | | | | | of |
| | | | | | predicted | | potential |
| DRSC | | CCE/ | basal/ | Z of | TM | | off- |
| amplicon | target gene | basal | Fmax | Fmax | segments | putative function | targets |
| DRSC11164 | Ets65A | 1.16 | 0.23 | −0.35 | 0 | transcription factor | 0 |
| DRSC04600 | Ca-P60A | 1.23 | 0.37 | 0.43 | 8 | SERCA pump | 0 |
| DRSC20158 | Stim | 1.26 | 0.28 | −1.03 | 1 | the putative ER Ca²⁺ | 0 |
| | | | | | | sensor for SOC | |
| | | | | | | activation | |
| DRSC04718 | Tsr | 1.28 | 0.37 | 0.56 | 0 | actin binding protein | 0 |
| DRSC02708 | cdc23 | 1.30 | 0.35 | −1.69 | 0 | component of | 1 |
| | | | | | | anaphase-promoting | |
| | | | | | | complex for mitotic | |
| | | | | | | anaphase | |
| DRSC22061 | Olf186-F | 1.31 | 0.29 | −1.11 | 4 | Drosophila CRAC | 0 |
| | | | | | | candidate | |
| DRSC04558 | Dom | 1.32 | 0.35 | 0.38 | 0 | component of | 0 |
| | | | | | | chromatin | |
| | | | | | | remodeling | |
| | | | | | | complex for DNA | |
| | | | | | | recombination | |
| DRSC03256 | Sec61alpha | 1.32 | 0.41 | 1.40 | 10 | component of | 0 |
| | | | | | | translocon complex | |
| | | | | | | for | |
| | | | | | | protein trafficking | |
| DRSC03432 | Syx5* | 1.33 | 0.33 | −2.21 | 1 | t-SNARE protein for | 0 |
| | | | | | | vesicle fusion | |
| DRSC18760 | deltaCOP | 1.34 | 0.32 | −1.39 | 0 | component of COPI | 0 |
| | | | | | | complex for | |
| | | | | | | protein trafficking | |

TABLE 2
Group 3 hits decreased CCE

| | | | | | number of |
|---|---|---|---|---|---|
| DRSC | | CCE/ | Basal/ | Z of | potential |
| amplicon | target gene | Basal | Fmax | Fmax | off-targets |
| DRSC00777 | Rab5 | 1.41 | 0.40 | 2.98 | 1 |
| DRSC02278 | CG13773 | 1.45 | 0.40 | −1.48 | 0 |
| DRSC03611 | smt3 | 1.36 | 0.37 | −1.69 | 0 |
| DRSC03342 | Hel25E | 1.40 | 0.30 | −1.08 | 0 |
| DRSC03574 | mts | 1.36 | 0.28 | 0.43 | 0 |
| DRSC03080 | Pvr | 1.37 | 0.39 | −1.58 | 0 |
| DRSC03256 | Sec61alpha | 1.32 | 0.41 | 1.40 | 0 |
| DRSC02179 | CG12750 | 1.35 | 0.31 | −1.91 | 0 |
| DRSC02708 | cdc23 | 1.30 | 0.35 | −1.69 | 1 |
| DRSC04600 | Ca-P60A | 1.23 | 0.37 | 0.43 | 0 |
| DRSC04558 | dom | 1.32 | 0.35 | 0.38 | 0 |
| DRSC08370 | CG13900 | 1.47 | 0.29 | −1.74 | 0 |
| DRSC07000 | Bap55 | 1.54 | 0.28 | 2.89 | 0 |
| DRSC07659 | pAbp | 1.38 | 0.34 | −1.75 | 0 |
| DRSC06044 | DMAP1 | 1.54 | 0.31 | 1.42 | 0 |
| DRSC11164 | Ets65A | 1.16 | 0.23 | −0.35 | 0 |
| DRSC11032 | CG8743 | 1.50 | 0.34 | 2.95 | 0 |
| DRSC11257 | Prosbeta2 | 1.52 | 0.33 | −1.55 | 0 |
| DRSC11124 | CycT | 1.47 | 0.33 | −1.66 | 4 |
| DRSC12536 | CG1249 | 1.54 | 0.27 | −1.65 | 0 |
| DRSC15625 | CG4699 | 1.55 | 0.32 | −0.17 | 0 |
| DRSC15948 | CG6015 | 1.55 | 0.30 | −1.53 | 0 |
| DRSC15166 | CG16941 | 1.53 | 0.28 | −1.67 | 0 |
| DRSC16034 | Dis3 | 1.42 | 0.30 | −1.52 | 0 |
| DRSC16839 | Rpn2 | 1.41 | 0.33 | −1.87 | 0 |
| DRSC18760 | deltaCOP | 1.34 | 0.32 | −1.39 | 0 |
| DRSC18360 | APC4 | 1.54 | 0.36 | −0.27 | 0 |
| DRSC20158 | Stim | 1.26 | 0.28 | −1.03 | 0 |
| DRSC00782 | RpL40 | 1.58 | 0.31 | −1.28 | 0 |
| DRSC03261 | CG9548 | 1.58 | 0.30 | −1.55 | 0 |
| DRSC02680 | CG18591 | 1.61 | 0.28 | −1.78 | 0 |
| DRSC02721 | Vha68-2 | 1.64 | 0.32 | 0.31 | 0 |
| DRSC02868 | Pect | 1.65 | 0.28 | 1.74 | 0 |
| DRSC04718 | tsr | 1.28 | 0.37 | 0.56 | 0 |
| DRSC04884 | Nipped-A | 1.54 | 0.36 | −1.17 | 0 |
| DRSC04838 | Bub1 | 1.59 | 0.36 | −1.38 | 0 |
| DRSC06417 | MrgBP | 1.56 | 0.34 | −1.42 | 0 |
| DRSC06421 | CG30349 | 1.59 | 0.32 | −1.73 | 0 |
| DRSC07501 | Pabp2 | 1.42 | 0.31 | −1.50 | 0 |
| DRSC07408 | E(Pc) | 1.48 | 0.34 | 2.04 | 0 |
| DRSC07575 | RacGAP50C | 1.62 | 0.26 | 2.70 | 0 |
| DRSC07583 | betaTub56D | 1.55 | 0.34 | −1.91 | 2 |
| DRSC07502 | hrg | 1.53 | 0.36 | 0.77 | 0 |
| DRSC08730 | pav | 1.55 | 0.34 | 1.31 | 1 |
| DRSC10696 | CG6694 | 1.58 | 0.31 | −1.59 | 0 |
| DRSC09740 | sti | 1.50 | 0.27 | 0.48 | 0 |
| DRSC11079 | CG9598 | 1.69 | 0.34 | 1.36 | 0 |
| DRSC11330 | brm | 1.54 | 0.33 | −1.38 | 0 |
| DRSC11663 | CG11451 | 1.52 | 0.34 | −1.10 | 0 |
| DRSC12351 | Gnf1 | 1.57 | 0.35 | −1.49 | 0 |
| DRSC12623 | alphaTub84D | 1.45 | 0.35 | −1.52 | 2 |
| DRSC14371 | CG31258 | 1.53 | 0.32 | −1.50 | 0 |
| DRSC16555 | bel | 1.56 | 0.30 | 3.39 | 3 |
| DRSC16899 | alphaTub85E | 1.39 | 0.37 | −0.46 | 3 |
| DRSC16940 | eff | 1.41 | 0.33 | −1.60 | 0 |
| DRSC16808 | Rab1 | 1.40 | 0.34 | −1.50 | 0 |
| DRSC16938 | eIF-3p66 | 1.41 | 0.36 | −1.65 | 0 |
| DRSC16704 | Hmgcr | 1.44 | 0.36 | −1.26 | 0 |
| DRSC16920 | cdc16 | 1.46 | 0.38 | −0.89 | 0 |
| DRSC18483 | Roc1a | 1.64 | 0.31 | −1.32 | 0 |
| DRSC18713 | Rpt4 | 1.37 | 0.34 | −0.97 | 0 |
| DRSC19385 | CG11138 | 1.50 | 0.30 | −0.21 | 3 |
| DRSC19570 | CG14214 | 1.51 | 0.33 | −0.69 | 1 |
| DRSC21306 | xmas-2 | 1.63 | 0.35 | −1.55 | 0 |
| DRSC05281 | E(Pc) | 1.56 | 0.34 | 3.86 | 0 |
| DRSC09005 | dpr6 | 1.47 | 0.29 | −1.54 | 2 |
| DRSC09132 | CycA | 1.57 | 0.29 | 1.24 | 0 |
| DRSC04725 | zip | 1.59 | 0.26 | 1.58 | 0 |
| DRSC18419 | dalao | 1.66 | 0.28 | 0.49 | 0 |
| DRSC21641 | CG40127 | 1.52 | 0.28 | 1.71 | 0 |
| DRSC21554 | Syx1A | 1.59 | 0.30 | 0.04 | 0 |
| DRSC21831 | swm | 1.66 | 0.29 | −1.12 | 0 |
| DRSC22061 | olf186-F | 1.31 | 0.29 | −1.11 | 0 |
| DRSC22489 | zip | 1.64 | 0.26 | 3.28 | 0 |
| DRSC23010 | Atx2 | 1.49 | 0.33 | 0.63 | 0 |

TABLE 3
Solutions for Ca²⁺ imaging and whole-cell recording.

| Name | Na+ | K+ | Ca2+ | Mg2+ | Cl− | HEPES | pH | Osmolality |
|---|---|---|---|---|---|---|---|---|
| S2 Ringer (Ca2) | 150 | 5 | 2 | 4 | 167 | 10 | 7.2 | 328 |
| Ca2+-free S2 Ringer (Ca0) | 150 | 5 | — | 6 | 167 | 10 | 7.2 | 332 |
| S2 external (Ca2) | 160 | — | 2 | — | 164 | 10 | 6.6 | 325 |
| High-Ca2+ S2 external | 124 | — | 20 | — | 164 | 10 | 6.6 | 324 |
| (Ca20) | | | | | | | | |
| Divalent free Na+ (Na) | 152 | — | — | — | 152 | 10 | 6.6 | 328 |
| Divalent free Cs+ (Cs) | 160 | — | — | — | 164 | 10 | 6.6 | 324 |

| | | | Mg2+ | | | |
|---|---|---|---|---|---|---|
| Name | Cs+ aspartate | CsCl | gluconate | HEPES | pH | Osmolality |
| S2 internal | 133 | 2 | 8 | 15 | 7.2 | 320 |

TABLE 4
Primers

| | | |
|---|---|---|
| | Primer | |
| Gene | name | Primer sequence 5′ to 3′ |

Drosophila dsRNA primers (T7 sequence underlined)

| | | |
|---|---|---|
| olf186-F | olf186-F- | GAATTAATACGACTCACTATAGGGAGAAT |
| | RNAi F1 | ACGAATGTACCACCGGG |
| | olf186-F- | GAATTAATACGACTCACTATAGGGAGACC |
| | RNAi R1 | AAGTGATGCTAGACAATGT |

Cloning primers

| | | |
|---|---|---|
| olf186-F | olf186-F- | CTGAACATGAAGCGGCCGCATCATGTCTG |
| | clone F1 | TGTGGACCAC |
| | olf186-F- | GCTGAACTCGAGCTAGACAATGTCCCCGG |
| | clone R1 | ATG |

RT-PCR Primers

| | | |
|---|---|---|
| olf186-F | olf186-F- | GAATTAATACGACTCACTATAGGGAGAAT |
| | RT F1 | ACGAATGTACCACCGGG |
| | olf186-F- | GAAAGAGTATGAGTCCCAGC |
| | RT R1 | |
| | olf186-F- | CCAACAATTCGGGCCTAGAGAC |
| | RT F2 | |
| | olf186-F- | GTAGGTGGGCGAGTGGAGATC |
| | RT R2 | |
| Stim | Stim-RT | CAGTGGAAGTGTTCAGGATCGC |
| | F1 | |
| | Stim-RT | CCACATCCATTGCCTTCAATGAG |
| | R1 | |
| CG11059 | CG11059- | CTCGCCTAGACTTATGTGAC |
| | RT F1 | |
| | CG11059- | CCAGTAGACCCATCAAAGTG |
| | RT R1 | |
| Presenilin | PSN-RT | CTACGGAGGCGAACGAACG |
| (Psn) | F1 | |
| | PSN-RT | GGCGATTGTTCATGGAAAGG |
| | R1 | |
| Ca-P60A | CaP60A- | CGATATCCGTATCACCCACA |
| | RT F1 | |
| | CaP60A- | CTCACCGAACTCGTCCAGTT |
| | RT R1 | |
| Syntaxin | Syx5-RT | CGCTTCCATTCCGACTAGTT |
| 5 (Syx5) | F1 | |
| | Syx5-RT | GCTTCTCCAGTTTTGCGTAG |
| | R1 | |
| tsr | Tsr-RT | GAAATGCGGACCTGGAGAGT |
| | F1 | |
| | Tsr-RT | CGACTTCTTGAGAGCATCGA |
| | R1 | |

In this example, our genome-wide screen, based upon direct Ca²⁺ influx measurements, validated Stim and identified several additional genes that are required for CRAC channel activity. We independently identified olf186-F (Orai) as essential for Ca²⁺ signaling and activation of CRAC current, confirming two recent reports (15, 21). In addition, we provide evidence based upon overexpression that it may form an essential part of the CRAC channel. In mammalian cells, overexpression of STIM1 increases Ca²⁺ influx rates and CRAC currents about two-fold (7, 8), but in S2 cells we show that overexpression of Stim alone does not increase CRAC current, consistent with Stim serving as a channel activator rather than the channel itself. In contrast, transfection of olf186-F by itself increased CRAC current densities three-fold; and co-transection of olf186-F with Stim resulted in an eight-fold enhancement and the largest CRAC currents ever recorded. These results support the hypothesis that olf186-F constitutes part of the CRAC channel and that Stim serves as the messenger for its activation. Consistent with this, the CRAC channel activation kinetics during passive Ca²⁺ store depletion were significantly faster with co-transfected Stim. Many details of the mechanism of CRAC channel activation remain to be clarified including the protein-protein interactions that underlie trafficking and channel activation. Site-directed mutagenesis in a heterologous expression system may help to define the putative pore-forming region.

Similar to Stim, knockdown of olf186-F did not produce a severe cell growth phenotype. It was neither a hit in a previous screen of cell survival (9) nor in any other published Drosophila whole-genome RNAi screen (10-14). The olf186-F gene is a member of a highly conserved gene family that contains three homologs in mammals, two in chicken, three in zebrafish, and one member only in fly and worm (FIG. 8A). C09F5.2, the only homolog in C. elegans, is expressed in intestine, hypodermis, and the reproductive system, as well as some neuron-like cells in the head and tail regions (www.wormbase.org). Worms under RNAi treatment against C09F5.2 are sterile (22). Analysis of hydrophobic regions of the predicted protein from the fly gene and the three mammalian homologs (FIG. 8B) suggested the presence of four conserved transmembrane segments. Cytoplasmic C termini are suggested by the presence of coiled-coil motifs in each sequence. A predicted transmembrane topology and the sequence for the fly gene are shown in FIG. 8C. Sequence alignment between members from human, chicken and fly revealed strong sequence conservation in putative transmembrane regions and conserved negatively charged residues in loops between transmembrane segments. All three human members are expressed in the immune system (GNF Symatlas http://symatlas.gnf.org/SymAtlas/). Mutation of a human homolog of Drosophila olf186-F, ORA11 on chromosome 12, appears to be the cause of defective CRAC channel activity in SCID patient T cells (15), consistent with a requirement for functional CRAC channels in the immune response. Interestingly, microarray data from public databases (GEO profiles, www.ncbi.nlm.nih.gov) combined with tissue-specific EST counts show that all three human members are expressed in a variety of non-excitable tissues including thymus, lymph node, intestine, dermis, and many other tissues including the brain; although expression patterns and levels are different among the three members.

Ca-P60A has been proposed to be the only Drosophila SERCA gene (23). We validated its ER pump function by showing that ionomycin did not induce significant store release from S2 cells pretreated with dsRNA against Ca-P60A, consistent with a previous report (23). The elevation in resting [Ca²⁺]ᵢ and rapidly changing Ca²⁺ transients during changes in external Ca²⁺ before addition of TG may indicate a low level of constitutive CRAC channel activity induced by store depletion. In addition, SERCA knockdown inhibited CRAC channel activity following passive store depletion in whole-cell patch recordings. These results are consistent with the SERCA pump being required for normal activity of CRAC channels, but do not rule out indirect inhibition of CRAC current as a consequence of residual high resting [Ca²⁺]ᵢ or store depletion. The role of SERCA in CRAC channel function merits further study.

Among the hits, several are known to be involved in protein trafficking. Both Syx5 and Syx1A's gene products are t-SNARE proteins involved in vesicle fusion in many cell types. We verified the RNAi effects of Syx5 at the single cell level and demonstrated strong suppression of CRAC channel activity as well as the SOC influx. tsr may regulate SOC influx indirectly by controlling cell metabolism since RNAi of tsr did not significantly influence CRAC current density in whole-cell patch clamp experiments. Membrane trafficking was previously suggested to be important for SOC channel activity in Xenopus oocytes, based upon inhibition by botulinum toxin or by a dominant-negative SNAP-25 construct (24); and our results further suggest a requirement for syntaxins and SNARE-complex formation, possibly to mediate translocation of Stim to the plasma membrane (6, 7). The screen also revealed three other groups of hits that may influence calcium dynamics. These results set the stage for experiments targeting specific genes to understand the fine tuning of Ca²⁺ homeostasis and signaling.

Additional details regarding this example can be found in Zhang et al. (2006) “Genome Wide RNAi Screen of Ca²⁺ influx identifies Genes that Regulate Ca²⁺ Channel Activity,” PNAS 103(24):9357-9362, incorporated herein by reference in its entirety.

### EXAMPLE REFERENCES

1. Prakriya, M. & Lewis, R. S. (2003) Cell Calcium 33, 311-21.
2. Parekh, A. B. & Putney, J. W., Jr. (2005) Physiol Rev 85, 757-810.
3. Gallo, E. M., Cante-Barrett, K. & Crabtree, G. R. (2006) Nat Immunol 7, 25-32.
4. Yeromin, A. V., Roos, J., Stauderman, K. A. & Cahalan, M. D. (2004) J Gen Physiol 123, 167-82.
5. Roos, J., DiGregorio, P. J., Yeromin, A. V., Ohlsen, K., Lioudyno, M., Zhang, S., Safrina, O., Kozak, J. A., Wagner, S. L., Cahalan, M. D., Velicelebi, G. & Stauderman, K. A. (2005) J Cell Biol 169, 435-45.
6. Liou, J., Kim, M. L., Heo, W. D., Jones, J. T., Myers, J. W., Ferrell, J. E., Jr. & Meyer, T. (2005) Curr Biol 15, 1235-41.
7. Zhang, S. L., Yu, Y., Roos, J., Kozak, J. A., Deerinck, T. J., Ellisman, M. H., Stauderman, K. A. & Cahalan, M. D. (2005) Nature 437, 902-5.
8. Spassova, M. A., Soboloff, J., He, L. P., Xu, W., Dziadek, M. A. & Gill, D. L. (2006) Proc Natl Acad Sci USA 103, 4040-5.
9. Boutros, M., Kiger, A. A., Armknecht, S., Kerr, K., Hild, M., Koch, B., Haas, S. A., Consortium, H. F., Paro, R. & Perrimon, N. (2004) Science 303, 832-5.
10. DasGupta, R., Kaykas, A., Moon, R. T. & Perrimon, N. (2005) Science 308, 826-33.
11. Agaisse, H., Burrack, L. S., Philips, J. A., Rubin, E. J., Perrimon, N. & Higgins, D. E. (2005) Science 309, 1248-51.
12. Baeg, G. H., Zhou, R. & Perrimon, N. (2005) Genes Dev 19, 1861-70.
13. Bard, F., Casano, L., Mallabiabarrena, A., Wallace, E., Saito, K., Kitayama, H., Guizzunti, G., Hu, Y., Wendler, F., Dasgupta, R., Perrimon, N. & Malhotra, V. (2006) Nature 439, 604-7.
14. Nybakken, K., Vokes, S. A., Lin, T. Y., McMahon, A. P. & Perrimon, N. (2005) Nat Genet 37, 1323-32.
15. Feske, S., Gwack, Y., Prakriya, M., Srikanth, S., Puppel, S. H., Tanasa, B., Hogan, P. G., Lewis, R. S., Daly, M. & Rao, A. (2006) Nature 441, 179-85.
16. Yagodin, S., Hardie, R. C., Lansdell, S. J., Millar, N. S., Mason, W. T. & Sattelle, D. B. (1998) Cell Calcium 23, 219-28.
17. Lepple-Wienhues, A. & Cahalan, M. D. (1996) Biophys J 71, 787-94.
18. Zweifach, A. & Lewis, R. S. (1996) J Gen Physiol 107, 597-610.
19. Christian, E. P., Spence, K. T., Togo, J. A., Dargis, P. G. & Patel, J. (1996) J Membr Biol 150, 63-71.
20. Redondo, P. C., Harper, M. T., Rosado, J. A. & Sage, S. O. (2006) Blood 107, 973-9.
21. Vig, M., Peinelt, C., Beck, A., Koomoa, D. L., Rabah, D., Koblan-Huberson, M., Kraft, S., Turner, H., Fleig, A., Penner, R. & Kinet, J. P. (2006) Science.
22. Maeda, I., Kohara, Y., Yamamoto, M. & Sugimoto, A. (2001) Curr Biol 11, 171-6.
23. Raymond-Delpech, V., Towers, P. R. & Sattelle, D. B. (2004) Cell Calcium 35, 131-9.
24. Yao, Y., Ferrer-Montiel, A. V., Montal, M. & Tsien, R. Y. (1999) Cell 98, 475-85.

### Example 2

### Molecular Identification of the CRAC Channel by Altered Ion Selectivity in a Mutant of Orai

Recent RNAᵢ screens have identified several proteins that are essential for store-operated Ca²⁺ influx and CRAC channel activity in Drosophila and in mammals, including the transmembrane proteins Stim^{1,2} and Orai³⁻⁵. Stim most likely functions as a sensor of luminal Ca²⁺ content and triggers activation of CRAC channels in the surface membrane following Ca²⁺ store depletion^{1,6}. Among three human homologs of Orai, Orai1 on chromosome 12 was found to be mutated in patients with severe combined immunodeficiency disease, and expression of the wild-type Orai1 restored Ca²⁺ influx and CRAC channel activity in patient T cells³. The overexpression of Stim and Orai together dramatically increases CRAC current^{5,7-9}. However, it was not yet clear whether Stim or Orai actually forms the CRAC channel, or if their expression simply limits CRAC channel activity mediated by a different channel-forming subunit. Here, we show that interaction between wild-type Stim and Orai, assessed by co-immunoprecipitation, is greatly enhanced following treatment with thapsigargin to induce Ca²⁺ store depletion. By site-directed mutagenesis, we show that a point mutation from glutamate to aspartate at position 180 in the conserved S1-S2 loop of Orai transforms the ion selectivity properties of CRAC current from being Ca²⁺-selective with inward rectification to being selective for monovalent cations and outwardly rectifying. A charge-neutralizing mutation at the same position (E to A) acts as a dominant-negative non-conducting subunit. Other charge neutralizing mutants in the same loop express large inwardly rectifying CRAC current, and two of these exhibit reduced sensitivity to the channel blocker Gd³⁺. These results indicate that Orai itself forms the Ca²⁺-selectivity filter of the CRAC channel.

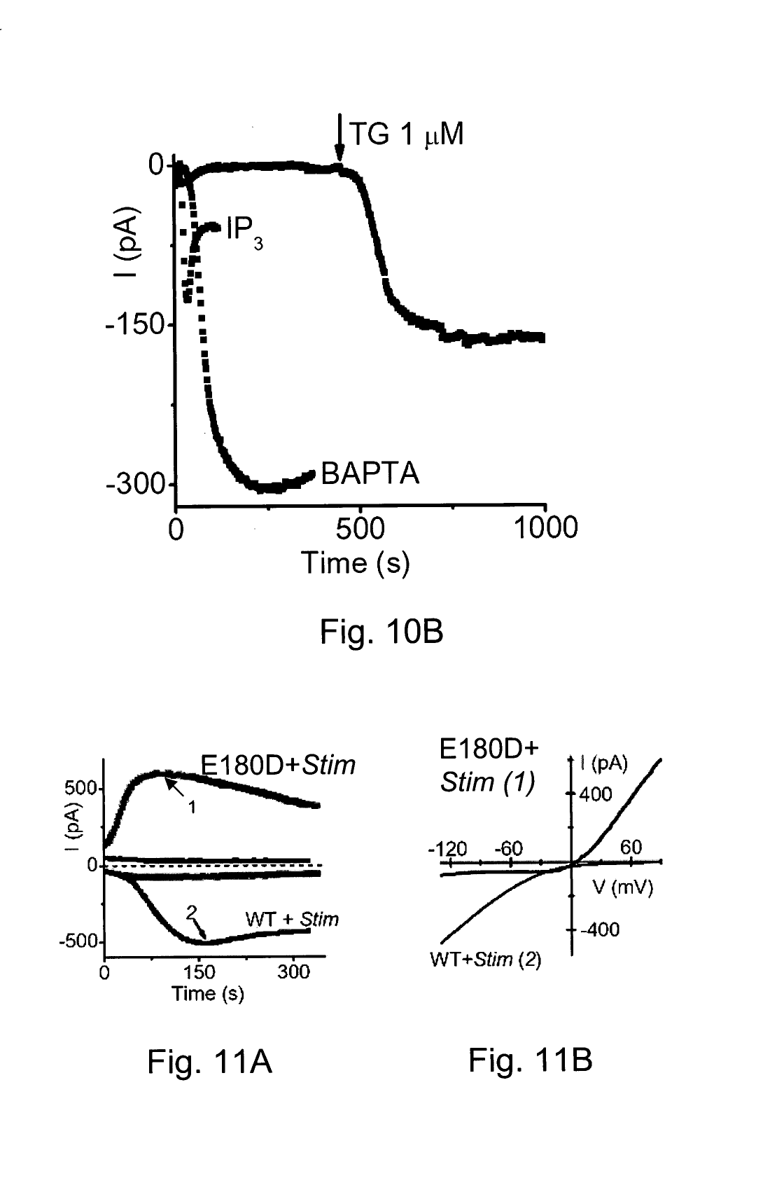

Orai and Orai1 possess four hydrophobic stretches that are predicted to span the membrane. Based upon strategies used previously for several different ion channels, we made point mutations to probe the most conserved loop between putative transmembrane segments (FIG. 10A) and examined properties of ion selectivity, current-voltage rectification and block that are intimately associated with a pore-forming subunit. Wild-type (WT) or mutant Orai were overexpressed together with wild-type Stim in S2 cells; mRNA and protein expression were verified by RT-PCR and western blotting (FIG. 10B; FIG. 10A). In addition, by co-immunoprecipitation of the epitope-tagged wild-type proteins, we evaluated whether Stim and Orai are associated with each other before and after Ca²⁺ store depletion. When cells were cultured without stimulation, only limited interaction was seen. However, Ca²⁺ store depletion triggered by thapsigargin (TG) to inhibit the SERCA reuptake pump dramatically increased Stim-Orai protein interaction when either protein was used to pull down the other by co-immunoprecipitation (FIG. 10a). This result shows that both transfected proteins are expressed and, moreover, that they interact, either directly or as part of a complex, following Ca²⁺ store depletion to initiate CRAC channel activation. During whole-cell recording on the co-transfected cells, dialysis of a Ca²⁺ chelator (BAPTA) into the cytoplasm activated a very large Ca²⁺-selective inward current, as shown previously⁵. Using a pipette solution that maintains normal Ca²⁺ store content to prevent spontaneous channel opening, we confirmed that addition of IP₃ inside or TG outside resulted in greatly augmented CRAC current (FIG. 10b).

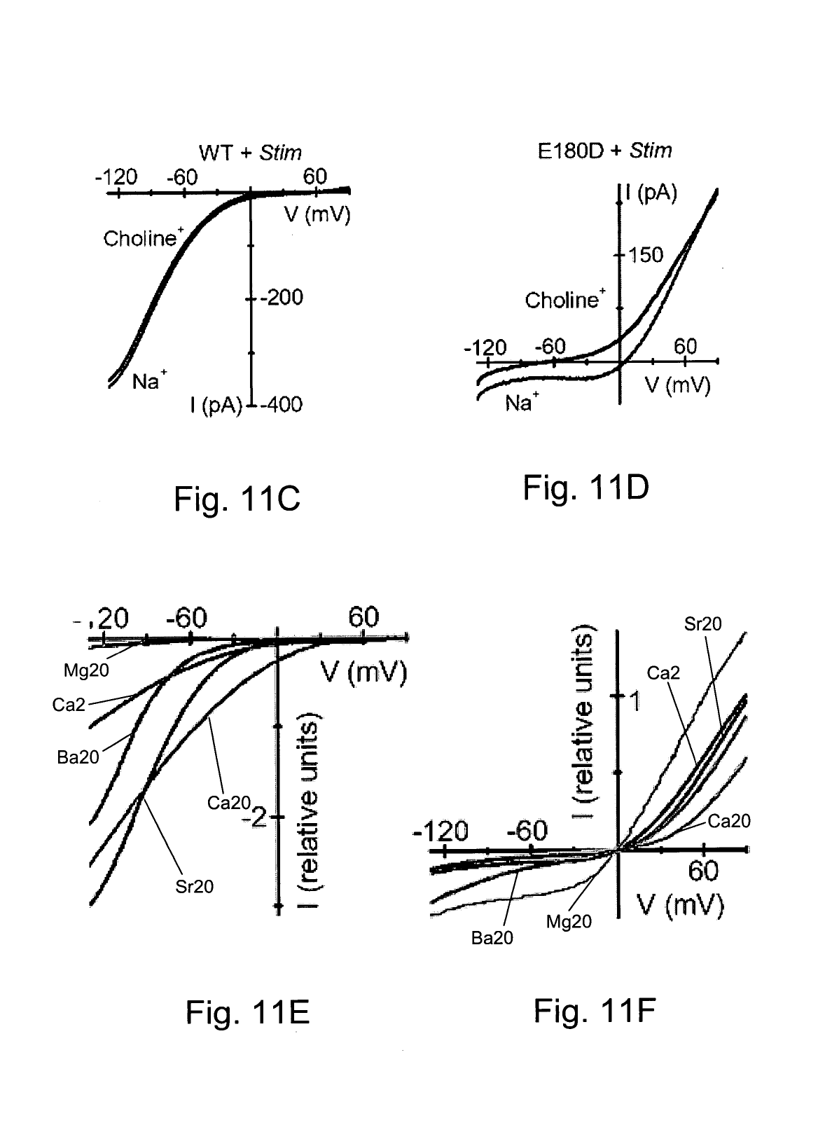

Expression of the Orai E180D mutant, again together with wild-type Stim, generated a current that developed with the normal kinetics of CRAC current during passive Ca²⁺ store depletion, but also exhibited dramatic alterations in ion selectivity and rectification. In contrast to the normal inward CRAC current, E180D Orai expression produced a very large outward current and a smaller inward current that developed in parallel (FIG. 11a). Current-voltage (I-V) curves of the E180D-induced current revealed an outwardly rectifying shape with a reversal potential of 6.0±0.4 mV (n=14 cells), in sharp contrast to the characteristic inwardly rectifying I-V curves with a reversal potential >50 mV of native or WT Orai-induced CRAC current (FIG. 11B).

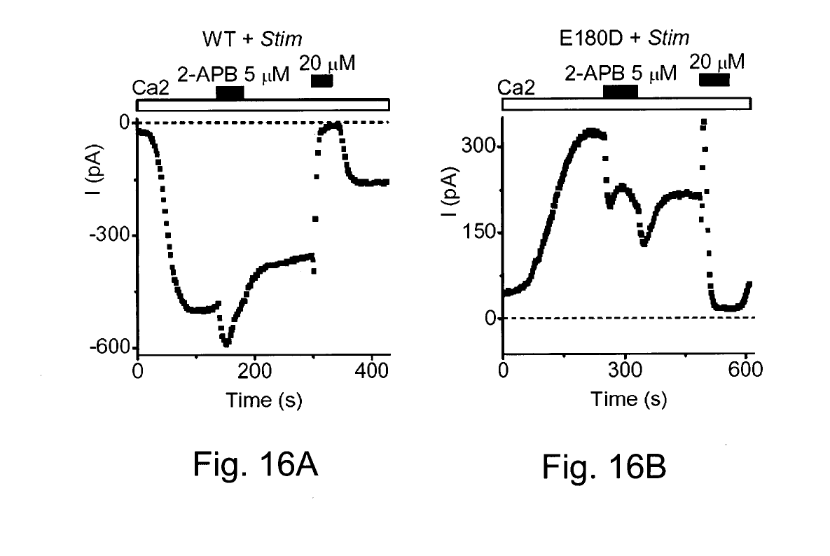

In a series of ion-substitution experiments, we evaluated the ion selectivity properties of current induced by E180D Orai. Substitution of Na⁺ by choline while maintaining constant Ca²⁺ in the external solution, an experimental maneuver that produces little effect on native CRAC current¹⁰ or CRAC current with overexpressed WT Orai (FIG. 11c), reduced the inward current and shifted the reversal potential to hyperpolarized potentials (FIG. 11d), indicating that the E180D-induced inward current is carried predominantly by Na⁺ rather than by Ca²⁺. Next, different divalent cation species in the external solution were tested, resulting in an I-V pattern of WT Orai-induced CRAC current similar to native CRAC current described previously¹⁰. Ca²⁺, Sr²⁺, and Ba²⁺ (20 mM in each case while maintaining high external Na⁺) resulted in increased inward current (FIG. 11e), indicating that each of these divalent cations is permeant, whereas Mg²⁺ is relatively impermeant. In contrast, representative I-V curves of E180D Orai-induced current, shown in FIG. 11f with the same color code for comparison with FIG. 11e, reveal an entirely different pattern. A ten-fold increase of [Ca²⁺] in the external solution significantly decreased the outward current and did little to the small inward current. Substitution of 2 mM external Ca²⁺ by 20 mM Sr²⁺ had little effect; Mg²⁺ increased the current; and Ba²⁺ significantly increased the inward current while decreasing the outward current (FIG. 15a). These results indicate that divalent cations block the outward current (carried by Cs⁺) in the potency sequence: Ca²⁺>Ba²⁺>Sr²⁺>Mg²⁺; and the inward current (carried by Na⁺) in the potency sequence: Ca²⁺˜Sr²⁺>Mg²⁺>Ba²⁺. Together, these ion-substitution experiments indicate that the E180D Orai mutation transforms the CRAC channel that is normally selective for divalent cations to a monovalent-selective channel that selects against, and indeed is blocked by, divalent cations.

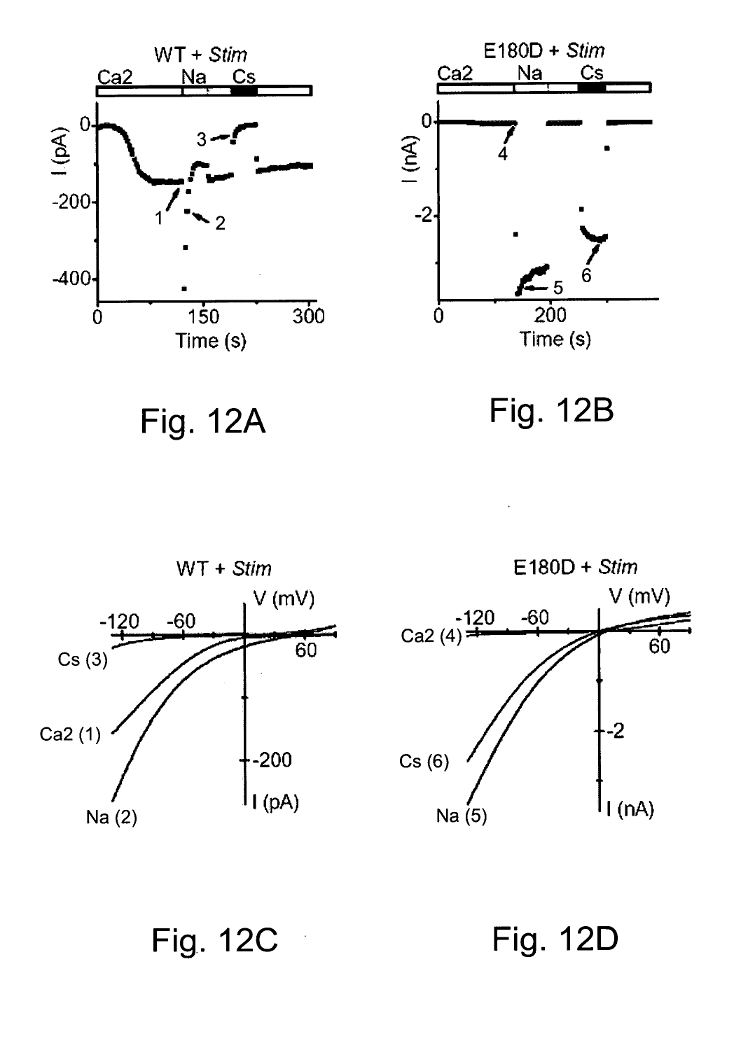

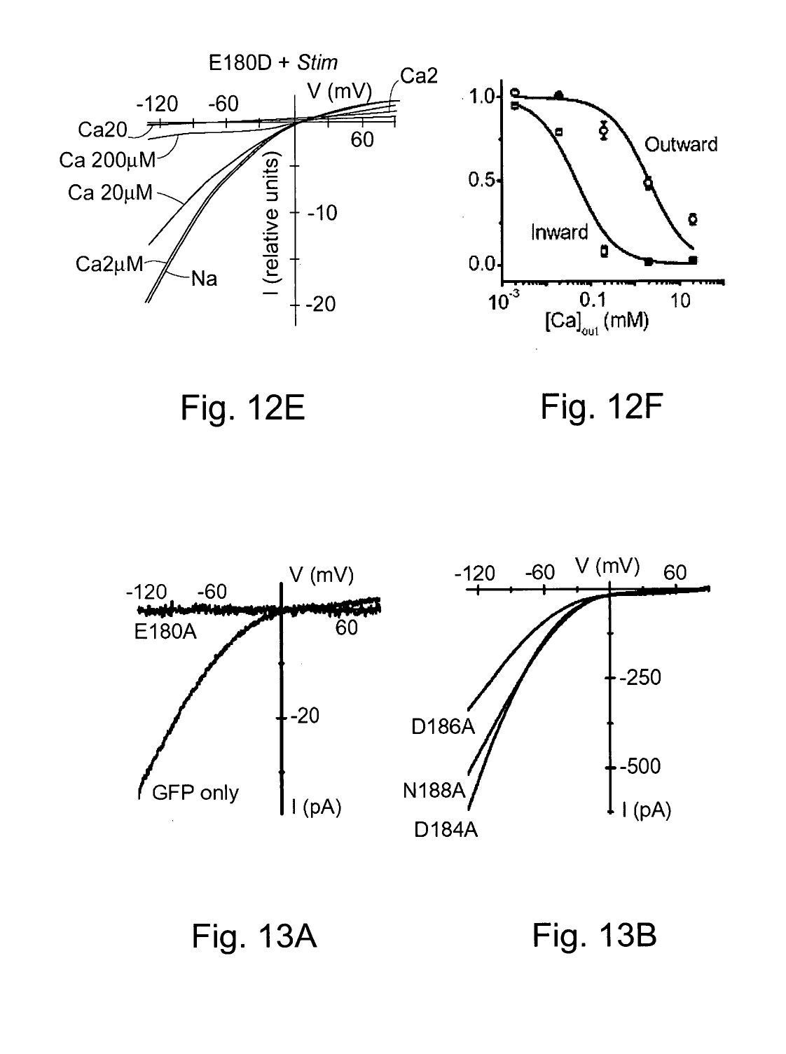

CRAC channels share with voltage-gated Ca²⁺ channels the property of monovalent ion permeation when external divalent ions are removed¹¹⁻¹⁵. Upon withdrawal of external divalent cations, Na⁺ carries significant current through CRAC channels, whereas Cs⁺ is far less permeant^{10,14,16,17}. The monovalent CRAC current normally declines within 10 sec, a process termed depotentiation¹⁵ that is due to removal of external Ca²⁺. To check whether the E180D mutation alters monovalent CRAC current, both WT and E180D Orai-induced CRAC current were recorded in divalent-free Na⁺ and Cs⁺ test solutions. Three clear differences can be discerned (FIG. 12). First, the inward Na⁺ current was much larger in the E180D mutant and did not depotentiate to the same extent as either WT Orai (FIGS. 12a and b) or native CRAC current in S2 cells¹⁰. Second, in contrast to the relatively small Cs⁺ current density in WT Orai (−1.6±0.6 pA/pF, n=4), E180D-induced Cs⁺ current density was much larger (−191±46 pA/pF, n=4), similar in amplitude to the Na⁺ current (−238±43 pA/pF, n=9); and the large Cs⁺ current did not depotentiate (FIG. 12b). Third, the measured reversal potential in Na⁺ external solution was 7.9±0.3 mV (n=9) for E180D, instead of 52.4±0.9 mV (n=5) for WT Orai (FIGS. 12c and d). Calculated from reversal potentials, the average permeability ratio P_{Na}/P_{Cs} has changed from 8.0 for WT Orai to 1.4 as a result of the E180D mutation, indicating an increase in pore diameter or altered electrostatic interactions with cations. I-V curves for E180D Orai-induced current changed shape with varying external [Ca²⁺] (FIG. 12e), revealing voltage-dependent block (FIG. 12f) that causes outward rectification in physiological saline. This examination of ion selectivity properties shows that a conservative mutation of glutamate to aspartate at position 180 of Orai transforms the CRAC channel from being Ca²⁺-selective and inwardly rectifying to one that conducts Na⁺ or Cs⁺ and is outwardly rectifying due to voltage-dependent Ca²⁺ block.

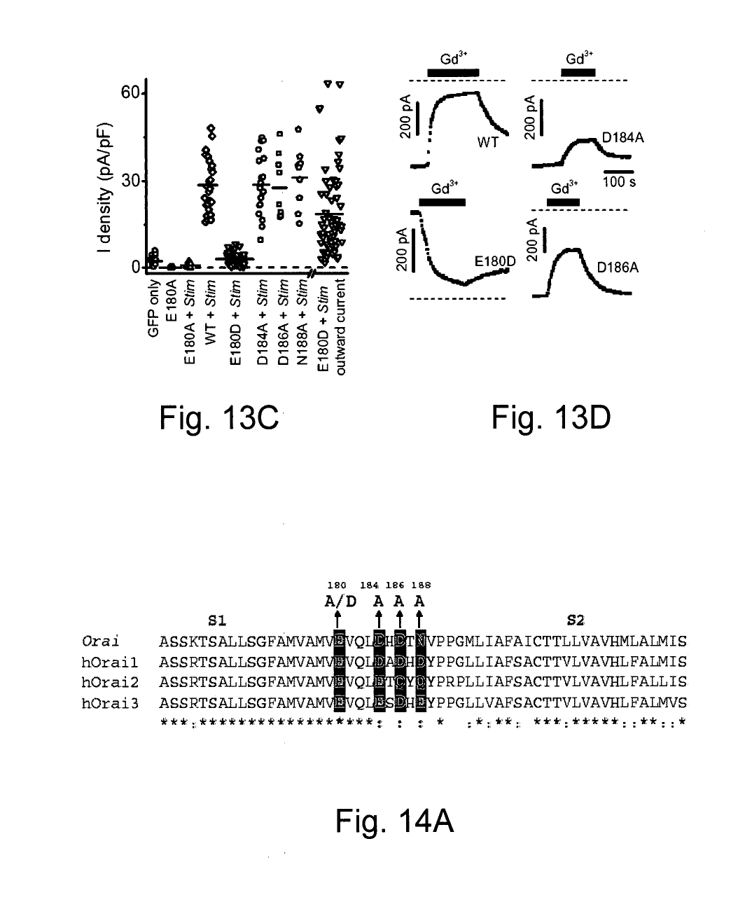

Expression of the charge-neutralizing E180A mutant of Orai completely abolished native CRAC current (FIG. 13a) suggesting that alanine-containing subunits prevent ion conduction in heteromultimeric CRAC channels by a dominant-negative action. In contrast, the expression of D184A, D186A, or N188A Orai mutants with Stim resulted in increased CRAC current with unaltered I-V shape (FIG. 13b) and normal monovalent Na⁺ current that exhibited depotentiation and low Cs⁺ permeability. FIG. 13c summarizes the effect of each Orai mutant tested on CRAC current density. Expressed by itself or together with Stim, only E180A potently inhibited native CRAC current. The inward current density and I-V shape in cells expressing D184A, D186A and N188A mutants did not differ significantly from current observed in cells expressing WT Orai. E180D was unique in producing very large outward currents as a result of altered ion selectivity.

Gd³⁺ very effectively suppresses native and WT Orai-induced CRAC current⁵. In addition to ion selectivity properties, block by Gd³⁺ was evaluated in all mutants (FIG. 13d). Two charge-neutralizing mutations, aspartate to alanine at positions 184 and 186 and, to a lesser degree, mutation of asparagine to alanine at position 188 significantly reduced the potency of Gd³⁺ block (FIG. 13d, FIG. 11b). As shown previously⁵, WT Orai CRAC current was potentiated by 2-aminoethyldiphenyl borate (2-APB) at a low concentration (5 μM) and inhibited at a high (20 μM) concentration (supplementary FIG. 3a), similar to effects of 2-APB on native CRAC current in Jurkat and in S2 cells^{10,18} Effects of 2-APB on E180D Orai-induced CRAC current were complex but still showed potentiation and inhibition (FIG. 12b). The time course of E180D outward CRAC current showed an immediate decrease preceding potentiation by 5 μM 2-APB, and an immediate increase preceding inhibition during 20 μM application (n=3). The pharmacological analysis provides support for the conclusion that the S1-S2 loop is involved in ion selectivity and block by Gd³⁺, and may contribute to the complex effects of 2-APB on CRAC current.

In summary, our results in this example demonstrate that TG-triggered store-depletion dynamically strengthens an interaction between Stim and Orai, supporting a model for CRAC channel activation in which Stim serves as the Ca²⁺ sensor to detect store depletion and as the messenger to activate CRAC channels in the plasma membrane. More importantly, we conclude that Orai is a bona fide ion channel, based on the following facts: 1) RNAi-mediated knock-down of Orai expression suppresses TG-dependent Ca²⁺ influx and CRAC channel activity; 2) overexpression of Orai with or without Stim augments CRAC currents that exhibit biophysical properties identical to native CRAC current; and 3) mutations of negatively charged residues within the putative pore region of Orai significantly alter ion selectivity, current rectification, and affinity to a charged channel blocker without altering channel activation kinetics. The dramatic alteration of these properties by a targeted point mutation provides definitive evidence that Orai embodies the pore-forming subunit of the CRAC channel.

The consensus sequence within the S1-S2 loop, EVQLD_D, contains the critical glutamate (highlighted in bold) shown here to control ion selectivity properties of the CRAC channel, and two aspartates (italicized) that may help to attract Gd³⁺ (and Ca²⁺) toward the pore. It is not similar to pore sequences found in other channels. Unlike the pore regions of voltage-gated Ca²⁺ (CaV) channels, which contain a relatively long loop and a ring of critical glutamates from different domains that form a high-affinity Ca²⁺-binding site¹⁹, the putative pore sequence of Orai is very short, and the key residue for ion selectivity (E180) is adjacent to the putative S1 segment. Nevertheless, since withdrawal of external divalent ions reveals permeability to monovalent cations in both CaV^{11,12} and CRAC channels^{10,14,16,17}, it is possible that the CRAC channel ion-selectivity filter is also formed by a ring of glutamates and that the mechanism of Ca²⁺ permeation is similar, although the single-channel conductance and maximum permeant ion size of the CRAC channel selectivity filter are smaller than that of the CaV channel^{10,17,20,21}. Negatively charged side chains also contribute to Ca²⁺ selectivity of TRPV6; in this instance aspartate (at position 541) is proposed to coordinate with Ca²⁺ ions and line the selectivity filter in a ring structure formed by four subunits²². The CRAC channel may be a multimer that includes several identical Orai subunits, since a non-conducting pore mutant (E180A) exerts a strong dominant-negative action on native CRAC current. Biochemical approaches and cysteine-scanning mutagenesis should be useful to better elucidate the unique pore architecture of the CRAC channel.

Molecular Cloning and Mutagenesis

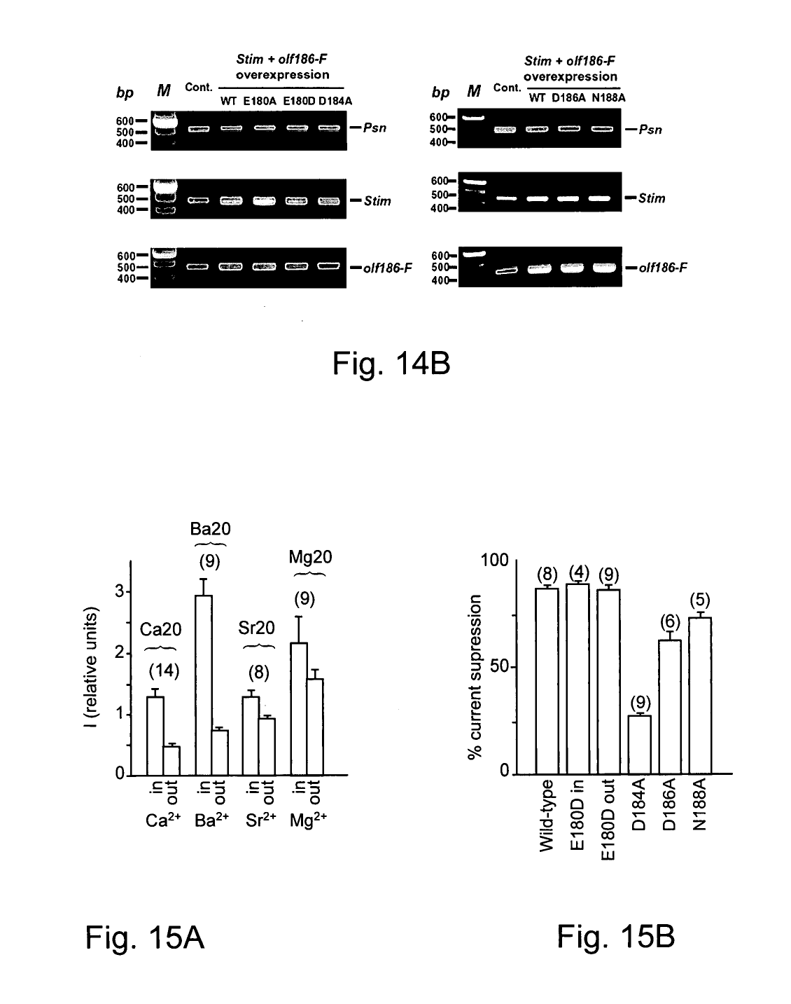

Generation of pAc5.1/EGFP, pAc5.1/D-STIM, pAc5.1/D-STIM-V5-His and pAc5.1/olf186-F (GenBank accession number DQ503470) were as described⁵. olf186-F pore mutants were made by exchanging the corresponding codons (E180A: GAG to GCG; E180D: GAG to GAC; D184A: GAT to GCT; D186A: GAT to GCT; N188A: AAT to GCT; FIG. 14) using the QuikChange site-directed mutagenesis kit (Stratagene). The pAc5.1/HA-olf186-F clone was made by adding the HA tag via PCR and re-cloned into the XhoI and NotI sites of pAc5.1/V5-His B expression vector. Resulting clones were confirmed by sequencing. Description of the primers and conditions for cloning is available upon request. Both HA-olf186-F and Stim-V5-His were verified for normal function by whole-cell recording.

RNA Isolation and RT-PCR

RNA was isolated using TRIZOL (Invitrogen) following the manufacturer's protocols. The methods for RT-PCR were the same as described⁶.

Cell Culture and Transfection

Drosophila S2 cells (Invitrogen) were propagated in Schneider's medium (Invitrogen) supplemented with 10% FBS at 24° C. Cells were seeded at a density of 10⁶ cells/ml and passaged when the cells achieved a density of ˜6×10⁶ cells/ml. S2 cells were transfected (see clones described in Methods) using a Nucleofector (Amaxa) following the manufacturer's protocol. Forty-eight hours post-transfection, cells were used for patch-clamp, biochemistry or processed for RT-PCR analysis.

Whole-Cell Recording

Cells transfected (Amaxa) with Orai and Stim constructs were identified by fluorescence of co-transfected GFP. Patch clamp experiments were performed at room temperature in the standard whole-cell recording, using conditions similar to those reported previously¹⁰. The membrane capacitance of S2 cells selected for recording was 11.6±0.4 pF (n=131 cells). Membrane potentials were corrected for a liquid junction potential of 10 mV between the pipette and bath solution. The series resistance (2-7 MΩ) was not compensated. The membrane potential was held at −10 mV, and 220-ms voltage ramps from −130 to 90 mV alternating with 220-ms pulses to −130 mV were delivered every 2 s. Only cells with high input (>2 GΩ) were selected for recording. External and pipette solution recipes are listed in supplementary table 1. Analyzed data are presented as mean ±SEM.

TABLE 5
Solutions for whole-cell recording

| | | Divalent/ | Ca²⁺ chelator/ | | |
|---|---|---|---|---|---|
| Name | Na⁺ | concentration | concentration | Cl⁻ | Sucrose |
| External (Ca2) | 160 | Ca²⁺/2 | — | 164 | — |
| Choline external (Chol) | 1.1 | Ca²⁺/2 | — | 164 | 10 |
| High-Ca²⁺ | 124 | Ca²⁺/20 | — | 164 | 10 |
| external (Ca20) | | | | | |
| High-Mg²⁺ | 124 | Mg²⁺/20 | — | 164 | 10 |
| external (Mg20) | | | | | |
| High-Sr²⁺ | 124 | Sr²⁺/20 | — | 164 | 10 |
| external (Sr20) | | | | | |
| High-Ba²⁺ | 124 | Ba²⁺/20 | — | 164 | 10 |
| external (Ba20) | | | | | |
| 200 μM Ca²⁺ (Ca 200 μM) | 160 | Ca²⁺/0.2 | — | 160 | — |
| 20 μM Ca²⁺ (Ca 20 μM) | 160 | Ca²⁺/0.825 | HEDTA/2 | 162 | — |
| 2 μM Ca²⁺ (Ca 2 μM) | 160 | Ca²⁺/0.85 | EGTA/2 | 162 | — |
| Divalent-free Na⁺ (Na) | 152 | — | HEDTA/10 | 152 | — |
| Divalent-free Cs⁺ (Cs) | — | — | HEDTA/10 | 160 | — |

| Name | Cs⁺ aspartate | Cs⁺ BAPTA | CsCl | HEPES |
|---|---|---|---|---|
| Ca²⁺-free internal | 133 | 12 | 2 | 15 |
| High-Ca²⁺ internal | 152 | — | — | 10 |

Solution names used in figures are indicated in bold. Concentrations of ions and chemicals in the table are indicated in mM. External solutions contained 10 mM D-glucose and 10 mM HEPES. Choline solution contained 160 mM of choline as choline chloride. Divalent-free Cs⁺ solution contained 160 mM of Cs⁺ as CsCl. Ca²⁺-free internal solution contained 8 mM Mg gluconate. High-Ca²⁺ internal solution contained 1 mM CaCl₂, 6 mM Ca(OH)₂ and 10 mM EGTA. pH of external and internal solutions was 6.6 and 7.2 respectively and was adjusted by appropriate hydroxide. pH of choline solution was adjusted by NaOH. Osmolality was adjusted to 324 mOsm±1% by sucrose. Low Ca²⁺ solutions (Ca 20 μM and Ca 2 μM) were composed using estimates of free Ca²⁺ concentration provided by MaxChelator v. 2.50 (http://maxchelator.stanford.edu/) using tables cmc0204e.tmc and the following parameter settings: t=23° C., pH=6.6, I=179 (for Ca 20 μM) or I=184 (for Ca 2 μM). Free Ca²⁺ concentration of high-Ca²⁺ internal solution estimated by MaxChelator was 450 nM. Gd³⁺ was added as GdCl₃. 2-APB was diluted from DMSO stock solution. IP₃ stock solution was prepared in water.

Data Analysis

Data were analyzed by Pulse (Heka Electronics) and Origin (OriginLab Corp.). Five I-V curves were averaged for display. Unless otherwise noted the displayed I-V curves are leak-subtracted. Since almost all S2 cells at the moment of break-in display an outward current that disappears during perfusion with internal solution (but before the inward CRAC current starts to develop), the following procedure for leak subtraction was employed. The first three ramp currents after the outward current subsided were averaged (I_{mid}). Then, another set of three ramps after maximal inward CRAC current had developed were averaged (Iₘₐₓ). The difference between Iₘₐₓ and I_{mid} represents an isolated CRAC current but with amplitude less than the actual one. To correct this the difference was scaled such that the value of current at −130 mV is equal to maximal inward current minus initial inward current. Then the scaled difference was subtracted from Iₘₐₓ followed by fitting with a polynomial function. This fit was considered as the basal leak current. In a few cases (e.g. FIG. 4b) the initial outward current did not run down until the maximal inward current had developed. In this case a linear I-V curve reversing at 0 mV with current magnitude at +90 mV equal to the sustained outward current was assigned as leak current. For E180D Orai-induced CRAC current, the initial current was considered as leak.

Co-Immunoprecipitation

Two days after transfection, 5×10⁶ cells were treated with either Ringer or zero-Ca²⁺ Ringer+1 μM TG using solutions described⁶ for 15 min at room temperature. Cells transfected with Stim-V5-His only or HA-olf186-F only were used as controls. Cell extracts were then prepared using RIPA lysis buffer (Upstate) according to manufacturer instructions. The extracts were pre-cleared with protein A/G beads (Pierce) and protein concentration was determined, using the Pierce BCA Protein Reagent Kit. The cell lysate was then diluted to approximately 1 mg/ml total protein with PBS and mixed with either anti-HA monoclonal antibodies (1 μg/100 μg total protein, Santa Cruz) or anti-V5 monoclonal antibodies (1 μg/100 μg total protein, Invitrogen) for 4 hr at 4° C. Equal amount of samples were mixed with either normal mouse IgG or vehicle as negative controls. Protein A/G beads were subsequently added (1 μl/1 μg IgG) to rock overnight at 4° C. Proteins were eluted by boiling in 4× sample buffer (Invitrogen). Samples were resolved by SDS-PAGE and analyzed by standard western blotting techniques. Anti-HA monoclonal antibodies (Santa Cruz) were used at a dilution of 1:200. Anti-V5 monoclonal antibodies (Invitrogen) were used at a dilution of 1:2500. Proteins were detected by developing with the SuperSignal (Pierce) detection system. Samples treated with normal mouse IgG or vehicle showed no detectable signal for both Stim-V5-His and HA-olf186-F (data not shown), indicating that the IP and co-IP are specific.

Additional Details

Additional details regarding this example can be found in Yeromin et al. (2006) “Molecular Identification of the CRAC Chanel by Altered Ion Selectivity in a Mutant of Orai” Nature 443(14):226-229, incorporated herein by reference in its entirety. In this example, the relevant glutamine residue is 180 in the Orai protein encoded by invitrogen S2 cell complementary DNA (GeneBank DQ503470, see also Zhang et al. (2006) “Genome Wide RNAi Screen of Ca²⁺ influx identifies Genes that Regulate Ca²⁺ Channel Activity,” PNAS 103(24):9357-9362, also incorporated herein by reference). The corresponding residue is 178 in the Drosophila genome database (AY071273) and 106 in the human Orai homologue (BC015369). Recently, other investigators have also identified Orai as an essential pore subunit of the CRAC channel, see Prakriya et al. (2006) “Orai1 is an essential pore subunit of the CRAC channel Nature on line advance publication, doi:10.1038/nature05122.

### REFERENCES

1. Liou, J. et al. STIM is a Ca²⁺ sensor essential for Ca²⁺-store-depletion-triggered Ca²⁺ influx. Curr Biol 15, 1235-41 (2005).
2. Roos, J. et al. STIM1, an essential and conserved component of store-operated Ca²⁺ channel function. J Cell Biol 169, 435-45 (2005).
3. Feske, S. et al. A mutation in Orai1 causes immune deficiency by abrogating CRAC channel function. Nature 441, 179-85 (2006).
4. Vig, M. et al. CRACM1 is a plasma membrane protein essential for store-operated Ca²⁺ entry. Science 312, 1220-3 (2006).
5. Zhang, S. L. et al. Genome-wide RNAi screen of Ca²⁺ influx identifies genes that regulate Ca²⁺ release-activated Ca²⁺ channel activity. Proc Natl Acad Sci USA 103, 9357-62 (2006).
6. Zhang, S. L. et al. STIM1 is a Ca²⁺ sensor that activates CRAC channels and migrates from the Ca²⁺ store to the plasma membrane. Nature 437, 902-5 (2005).
7. Mercer, J. C. et al. Large store-operated calcium-selective currents due to co-expression of Orai1 or Orai2 with the intracellular calcium sensor, Stim1. J Biol Chem (2006).
8. Peinelt, C. et al. Amplification of CRAC current by STIM1 and CRACM1 (Orai1). Nat Cell Biol (2006).
9. Soboloff, J. et al. Orai1 and STIM reconstitute store-operated calcium channel function. J Biol Chem (2006).
10. Yeromin, A. V., Roos, J., Stauderman, K. A. & Cahalan, M. D. A store-operated calcium channel in Drosophila S2 cells. J Gen Physiol 123, 167-82 (2004).
11. Almers, W. & McCleskey, E. W. Non-selective conductance in calcium channels of frog muscle: calcium selectivity in a single-file pore. J Physiol 353, 585-608 (1984).
12. Hess, P. & Tsien, R. W. Mechanism of ion permeation through calcium channels. Nature 309, 453-6 (1984).
13. Hoth, M. & Penner, R. Calcium release-activated calcium current in rat mast cells. J Physiol 465, 359-86 (1993).
14. Lepple-Wienhues, A. & Cahalan, M. D. Conductance and permeation of monovalent cations through depletion-activated Ca²⁺ channels (ICRAC) in Jurkat T cells. Biophys J 71, 787-94 (1996).
15. Zweifach, A. & Lewis, R. S. Calcium-dependent potentiation of store-operated calcium channels in T lymphocytes. J Gen Physiol 107, 597-610 (1996).
16. Kozak, J. A., Kerschbaum, H. H. & Cahalan, M. D. Distinct properties of CRAC and MIC channels in RBL cells. J Gen Physiol 120, 221-35 (2002).
17. Bakowski, D. & Parekh, A. B. Monovalent cation permeability and Ca²⁺ block of the store-operated Ca²⁺ current I_{CRAC} in rat basophilic leukemia cells. Pflugers Arch 443, 892-902 (2002).
18. Prakriya, M. & Lewis, R. S. Potentiation and inhibition of Ca²⁺ release-activated Ca²⁺ channels by 2-aminoethyldiphenyl borate (2-APB) occurs independently of IP₃ receptors. J Physiol 536, 3-19 (2001).
19. Ellinor, P. T., Yang, J., Sather, W. A., Zhang, J. F. & Tsien, R. W. Ca²⁺ channel selectivity at a single locus for high-affinity Ca²⁺ interactions. Neuron 15, 1121-32 (1995).
20. Prakriya, M. & Lewis, R. S. Separation and characterization of currents through store-operated CRAC channels and Mg²⁺-inhibited cation (MIC) channels. J Gen Physiol 119, 487-507 (2002).
21. Zweifach, A. & Lewis, R. S. Mitogen-regulated Ca²⁺ current of T lymphocytes is activated by depletion of intracellular Ca²⁺ stores. Proc Natl Acad Sci USA 90, 6295-9 (1993).
22. Voets, T., Janssens, A., Droogmans, G. & Nilius, B. Outer pore architecture of a Ca²⁺-selective TRP channel. J Biol Chem 279, 15223-30 (2004).
23. Prakriya et al. (2006) “Orai1 is an essential pore subunit of the CRAC channel Nature on line advance publication, doi:10.1038/nature05122.

While the foregoing invention has been described in some detail for purposes of clarity and understanding, it will be clear to one skilled in the art from a reading of this disclosure that various changes in form and detail can be made without departing from the true scope of the invention. For example, all the methods, compositions and systems described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

1. A recombinant cell comprising a heterologous orai gene and a heterologous stim gene.

2. The recombinant cell of claim 1, wherein the cell is a mammalian, human, rodent, insect, or Xenopus cell.

3. The recombinant cell of claim 1, wherein the orai gene and the stim gene are human.

4. The recombinant cell of claim 1, wherein the orai gene and the stim gene are expressed in the cell to produce heterologous Orai and heterologous Stim polypeptides.

5. The recombinant cell of claim 1, wherein the heterologous orai gene and the heterologous stim gene are expressed in the cell and a heterologous Orai/Stim polypeptide complex is formed in the cell, or in a membrane of the cell.

6. An isolated or recombinant polypeptide complex comprising at least one of: a recombinant Orai polypeptide or a recombinant Stim polypeptide, the complex further comprising at least one of: an Orai polypeptide, a Stim polypeptide, a recombinant Orai polypeptide or a recombinant Stim polypeptide.

7-10. (canceled)

11. A knock out non-human animal comprising a defect in a native orai gene or a defect in native orai gene expression, or both.

12-15. (canceled)

16. A method of identifying a compound that binds to or modulates an activity of an Orai polypeptide, or an Orai polypeptide/Stim polypeptide complex, the method comprising:
(a.) contacting a biological or biochemical sample comprising the polypeptide or complex with a test compound; and,
(b.) detecting binding of the test compound to the polypeptide or complex, or modulation of the activity of the polypeptide or polypeptide complex by the test compound, thereby identifying the compound that binds to or modulates the activity of the polypeptide or complex.

17-37. (canceled)

38. A system for detecting compounds that bind to or modulate an activity of an Orai polypeptide or Orai/Stim polypeptide complex, the system comprising:
(a.) a biological sample comprising the polypeptide or the polypeptide complex;
(b.) a source of a plurality of test compounds; and,
(c.) a detector capable of detecting binding of one or more of the test compounds to the polypeptide or polypeptide complex, or modulation of the activity of the polypeptide or complex by one or more of the test compounds, thereby identifying a compound that binds to or modulates the activity of the polypeptide or complex.

39-43. (canceled)

44. A recombinant cell that expresses a heterologous orai gene.

45-46. (canceled)

47. A method of detecting a molecular basis for an orai gene abnormality, the method comprising:
determining whether a biological sample from a patient comprises a polymorphism in a gene encoding Orai or an abnormality in expression of Orai; and,
correlating the polymorphism with an abnormality.

48. (canceled)

49. A method of rescuing a cell that has altered or missing Orai function, comprising introducing a nucleic acid into the cell, wherein the nucleic acid encodes a recombinant polypeptide homologous to a natural Orai polypeptide, and expressing the recombinant polypeptide, thereby providing Orai function to the cell.

50-51. (canceled)

52. An antibody that binds to an Orai polypeptide or to an Orai-Stim polypeptide complex.

53. A mutant Orai polypeptide that has an alteration in ion selectivity.

54-59. (canceled)

60. A method of identifying a gene that regulates CRAC channel activity in a cell comprising:
(a) contacting the cell with an agent that inhibits SERCA pump-mediated reuptake of Ca²⁺ into cellular stores;
(b) contacting the cell with an RNAi molecule; and
(c) detecting a change in Ca²⁺ influx as compared to Ca²⁺ influx of the cell prior to contacting with the agent, wherein a reduction in Ca²⁺ influx is indicative of a gene that regulates CRAC channel activity.

61-63. (canceled)

64. A method of identifying an agent that blocks calcium flux through a calcium channel gene comprising:
(a) contacting a cell containing a calcium channel gene with an agent;
(b) contacting the cell with an RNAi molecule; and
(c) detecting a change in calcium flux as compared to calcium flux of the cell prior to contacting with the agent, wherein a reduction in calcium flux is indicative of an agent that blocks calcium flux through a calcium channel gene.

65-67. (canceled)

68. A method of treating an immunological disorder in a subject comprising administering to the subject a therapeutically effective amount of a protein identified as a SOC regulator, thereby increasing the Ca²⁺ influx in cells of the subject.

69-71. (canceled)

72. A method of treating a cell proliferative disorder comprising administering to a subject in need thereof, a therapeutically effective amount of a protein identified as a SOC regulator, thereby increasing the Ca²⁺ influx in cells of the subject.

73-75. (canceled)
